(19) United States
(12) Patent Application Publication
(10) Pub. No.: US 2023/0313325 A1
(43) Pub. Date: Oct. 5, 2023

(54) METHODS FOR DETECTION OF PATHOGENIC INFECTIONS USING RED BLOOD CELL-CONTAINING PATIENT SAMPLES

(71) Applicant: The Trustees of the University of Pennsylvania, Philadelphia, PA (US)

(72) Inventor: Nilam Mangalmurti, Bryn Mawr, PA (US)

(21) Appl. No.: 17/997,742

(22) PCT Filed: May 7, 2021
(86) PCT No.: PCT/US2021/031323

§ 371 Date: Nov. 2, 2022

Related U.S. Application Data
(60) Provisional application No. 63/022,181, filed on May 8, 2020.

(51) Int. Cl. C12Q 1/689; C12Q 1/70

(52) U.S. Cl. CPC C12Q 1/701; C12Q 1/689; C12Q 1/6851; C12Q 2600/16

(57) ABSTRACT

Described herein are methods of diagnosing a pathogenic infection in a subject. The method includes contacting a red blood cell-containing sample from the subject with a reagent capable of detecting the pathogen in the sample; and diagnosing the subject with a pathogenic infection when the pathogen is detected in the sample. In some embodiments, suitable samples are less than 10µL.

### STATEMENT REGARDING FEDERALLY SPONSORED RESEARCH

This invention was made with government support under HL 126788 awarded by the National Institutes of Health. The government has certain rights in the invention.

### BACKGROUND OF THE INVENTION

Respiratory infections are a substantial cause of death globally, and sepsis, the dysregulated host response to infection, is a dreaded complication of pneumonia and the leading cause of death in US hospitals.² Early appropriate antibiotic treatment is essential for survival as every hour delay in antibiotic therapy increases mortality. However, blood cultures are positive in less than 20% of respiratory infections, and respiratory samples are often unattainable from patients. Because of the current setbacks with blood culture, including long turnaround time and limited utility in patients who have been started on antibiotics, there has been heightened interest in molecular tests including NGS (next generation sequencing) and Taqman based PCR assays for the diagnoses of bacterial infection and pneumonia. However, despite initial enthusiasm and over a decade of investment in the development of these tests, molecular diagnostics have not been successfully adapted for wide-spread use due to the lack of sensitivity of these tests and high costs. A need in the art exists for improved methods for detecting pathogenic infections and complications relating therefrom, in patient samples.

### SUMMARY OF THE INVENTION

Provided herein, in a first aspect, is a method of diagnosing a pathogenic infection in a subject. In one embodiment, the method includes contacting a red blood cell-containing sample from the subject with a reagent capable of detecting a pathogen-associated molecule in the sample; and diagnosing the subject with a pathogenic infection when the pathogen-associated molecule is detected in the sample. In one embodiment, the reagent is pathogen specific. The pathogen is selected from a bacteria, virus, mycobacterium, parasite, or plasmodium. In one embodiment, the sample is a blood sample containing RBCs from the subject which is free from culturable pathogen, e.g., bacteria, virus, mycobacterium or parasite. In another embodiment, the is substantially free from all other blood components other than RBCs. In one embodiment, the sample volume is about 1 µL to about 10 µL.

In one embodiment, the pathogenic infection is a bacterial infection, and the reagent is capable of detecting bacterial DNA in the sample; and the subject is diagnosed with a bacterial infection when bacterial DNA is detected in the sample. In some embodiments, the method further includes treating the subject for the bacterial infection when diagnosed with the same.

In another embodiment, the pathogenic infection is a viral infection, and the reagent is capable of detecting viral DNA or RNA in the sample; and the subject is diagnosed with a viral infection when viral DNA or RNA is detected in the sample. In one embodiment, the method includes treating the subject for the viral infection when diagnosed with the same.

In another embodiment, the pathogenic infection is a parasitic infection, and the reagent is capable of detecting parasite DNA in the sample; and the subject is diagnosed with a parasitic infection when parasite DNA is detected in the sample. In one embodiment, the method includes treating the subject for the parasitic infection when diagnosed with the same.

In another aspect, a method of detecting complement activation in a subject is provided. In one embodiment, the method includes contacting a RBC containing sample with a reagent capable of identifying a complement protein, or fragment thereof, and diagnosing the subject with compliment activation when a compliment protein or fragment thereof is detected on the RBCs. In one embodiment, the subject has, or is suspected of having, a pathogenic infection. In one embodiment, the pathogenic infection is COVID19. In another embodiment, the method further includes treating the subject for complement dysregulation when compliment is detected on the RBCs.

In other aspects, compositions and kits for performing the methods described herein are provided.

### BRIEF DESCRIPTION OF THE DRAWINGS

FIG. 1A - FIG. 1F shows DNA binds mammalian erythrocytes through surface expressed TLR9. (FIG. 1A) Non-permeabilized, unfixed erythrocytes from a healthy human donor and mouse were labeled with TLR9 ectodomain specific antibodies and analyzed by flow cytometry. (FIG. 1B) Confocal images showing surface expression of TLR9 and PKH 26 dye-labelling of human and murine red cell membranes. (FIG. 1C) RBCs from healthy human donors were incubated with increasing doses of Legionella DNA (0.1, 1, 10 ng), and presence of bacterial DNA was quantified by quantitative PCR of 16S amplification (top panel). 3 individual studies, *P=0.008, ANOVA. Amplicons from one representative study are shown (bottom panel). Lane 1: marker. (FIG. 1D) Increasing amounts of RBCs (10⁶, 10⁷, 10⁸) were incubated with PBS or medium of a P. falciparum positive culture (CM), and parasite DNA binding was quantified by DNA extraction and P. falciparum mtDNA amplification. Optical density (OD) quantification of P. falciparum DNA bound to RBCs is shown (top panel). P═NS for 10⁶ RBCs, *P<0.04 for 10⁷ RBCs, and *P<0.02 for 10⁸ RBCs, t-test comparing PBS v CM treated RBCs performed. RBCs from 3 individual donors were tested in 3 independent experiments. Amplicons from one representative study (bottom panel) is shown. Lane 1: marker. (FIG. 1E) RBC binding to synthetic Plasmodium CpG was analyzed using flow cytometry. RBCs from 5 healthy donors were tested with 2 doses of CpG. Representative results from one donor is shown. (FIG. 1F) Corresponding summary statistics. Each line represents a different donor. n=5 donors, *P=0.002, Kruskall- Wallis ANOVA, Multiple Comparisons (Tukey), *P=0.002 no CpG v 1 uM, 1 uM v 100 nM, P=0.234, 100 nM v no CpG, P=0.157.

FIG. 2A — FIG. 2L show RBCs undergo structural alterations upon CpG binding. (FIG. 2A) Osmotic fragility of healthy human RBCs pre-treated with PBS, 100 nM CpG, or 1 uM CpG was determined using hemolysis assay. (FIG. 2B) Hemolysis of RBCs pre-treated with PBS, 100 nM CpG, or 1 uM CpG after incubating cells in water. RBCs from 6 independent donors were tested. *P=0.02, Kruskal- Wallis, one-way ANOVA, Multiple Comparisons (Dunns) *P=0.022 no CpG v 1uM, P=0.132 no CpG v 100 nM. (FIG. 2C — FIG. 2E) RBCs were treated with CpG and labeled with TLR9 antibody. (FIG. 2C) Imaging flow cytometry reveals smooth and altered RBC populations as defined by Mean Pixel and Intensity parameters. (FIG. 2D) Smooth and altered RBC populations were analyzed for CpG binding and TLR9 expression. (FIG. 2E) Images of smooth and altered RBCs displaying differences in CpG and surface TLR9 detection. (FIG. 2F — FIG. 2G) RBCs incubated with increasing doses of CpG were analyzed for (FIG. 2F) RBC alteration (FIG. 2G) and TLR9 positivity. (FIG. 2H) Electron micrograph of RBCs following CpG incubation. (FIG. 2I) Quantification of RBC alteration in untreated and CpG-treated RBCs observed by electron microscopy. Alteration is defined by loss of biconcave disk shape and formation of echinocytes. Cells from 5 separate fields were counted and averaged. *P<0.001. (FIG. 2J) Confocal images showing RBC cytoskeletal proteins actin and spectrin following CpG treatment. (FIG. 2K) Confocal imaging displays Band 3 distribution following CpG and control GpC treatments. (FIG. 2L) Quantification of RBC alteration by Band3 staining displays CpG treated cells are more altered than GpC treated cells and untreated cells. RBCs from 4 individual donors tested. *P=0.010 for all three groups (untreated, CpG, and GPC) by one-way ANOVA and *P=0.034, RBC v CpG, post-hoc Dunn’s test.

FIG. 3A — FIG. 3J show DNA binding by surface expressed TLR9 induces RBC loss of self. (FIG. 3A) RBCs pre-loaded with Calcein AM cell permeant dye were treated with high-dose CpG or calcium ionophore A23187 to determine cell viability by flow cytometry. (FIG. 3B) Mean fluorescent intensity (MFI) of Calcein AM in RBCs pre-loaded with Calcein AM and treated with high-dose CpG or calcium ionophore, n=3 donors, *P<0.001, t test untreated RBC v ionophore, P═NS, untreated RBCs v CpG. (FIG. 3C) Flow cytometry analysis of phosphatidylserine (PS) externalization on RBCs incubated without or with fluorescent CpG (100 nM, 1 uM) prior to Annexin V staining. (FIG. 3D) Average PS externalization, normalized to untreated cells, following CpG treatment, n=4 healthy donors. (FIG. 3E) CD47 staining following CpG treatment. RBCs from 5 independent donors were tested, results from 1 donor are shown. (FIG. 3F) MFI of CpG after gating on CD47 positive and CD47 negative cells detected by the anti-CD47 CC2C6 antibody. Each pair represents RBCs from one donor, gated on CD47 positive v CD47 negative cells. Results are shown for 5 donors. *P=0.04, paired t-test. (FIG. 3G) RBCs from healthy donors were treated with CM or DNase pre-treated CM, and differences in CD47 expression were determined by flow cytometry. Four RBC preparations were tested, with 3 of 4 demonstrating CD47 loss following exposure to P. falciparum CM that was partially abrogated by DNase treatment. Data from one donor are displayed. (FIG. 3H) P. falciparum PCR to assess the effect of DNase treatment of two different culture media. M: marker. (FIG. 3I) CD47 detection by CC2C6 antibody on healthy RBCs following incubation with CM from an uninfected culture or CM from a P. falciparum culture. (FIG. 3J) CD47 expression on RBCs from 6 healthy donors following incubation with CM from a P. falciparum culture. For 3 donors, CD47 expression was tested using two different CM preparations (denoted by triangles). *P=0.034 Mann Whitney, *P=0.054 one-tailed t-test.

FIG. 4A - FIG. 4E show TLR9 dependent CD47-loss and RBC clearance. (FIG. 4A) CD47 expression on murine RBCs following CpG treatment was analyzed by flow cytometry, n=10 WT mice, representative data from one mouse is shown. (FIG. 4B) RBCs from WT and TLR9 KO mice were treated without or with CpG and analyzed for CD47 expression. Each line represents RBCs from an individual mouse. For WT, two-tailed paired t-test *P=0.022. For KO, not significant. For non-CpG treated WT v TLR9 KO, *P=0.013 Mann Whitney (n=6-9 mice/group, 3 independent experiments). (FIG. 4C — FIG. 4E) WT and TLR9 KO RBCs were labeled separately with different PKH colors and combined (mixed at 1: 1 ratio) prior to treatment with CpG DNA. WT mice were transfused with the PKH labeled WT and TLR9 KO erythrocytes. (FIG. 4C) Flow cytometry reveals a difference in detection between WT and TLR9 KO RBCs remaining in the circulation of transfused mice. (FIG. 4D) The ratio of TLR9 KO/WT RBCs in the circulation of transfused mice at all measured time points, *P< 0.008. Each circle represents an individual mouse. (FIG. 4E) Percent of KO and WT RBCs in circulation at 60 minutes for each mouse. Each pair of connected dots represents an individual mouse (n=5 mice, 2 independent experiments), *P<0.001 for every timepoint tested, t test or Mann-Whitney where appropriate.

FIG. 5A — FIG. 5F demonstrate that CpG is increased on RBCs during infection and CpG-RBCs undergo increased erythrophagocytosis. (FIG. 5A) mitochondrial DNA (mtCol) on plasma and RBCs from mice subjected to bacterial, viral and parasitic infection (right panel) or sepsis (CLP) and sterile inflammation (sham surgery), *P<0.001, paired t-test for RBCs v plasma during infection and *P=0.002 for rbcs v plasma in naïve mice (right panel). For the sepsis model, *P<0.001 RBCs v plasma for CLP and naïve mice, *P=0.002 for Sham mice, paired t test. (FIG. 5B) mtDNA on RBCs from naïve, sham, and CLP mice, *P=0.037, Kruskal-Wallis test, post-hoc Dunn’s test, *P=0.024 sham v naïve, P=0.089 CLP v naïve. (FIG. 5C — FIG. 5E) Green fluorescent protein expressing RBCs were treated with PBS or CpG, followed by transfusion into WT mice for 6 hours. Splenic red pulp macrophages (RPMs) were identified as being CD3-, CD45+, and F4/80+. (FIG. 5C) Gating and representative data of GFP positivity in RPMs. (FIG. 5D) GFP positivity of RPM for each individual mouse (n=6 mice/group, 2 independent experiments). *P=0.015, t-test. (FIG. 5E) MFI of GFP in GFP⁺ RPMs *P=0.023, t-test. (FIG. 5F) Spleen weights of mice transfused with PBS- or CpG-treated GFP-RBCs at 20 hours post-transfusion. *P=0.013, t-test.

FIG. 6A — FIG. 6G show CpG-carrying RBCs induce systemic inflammatory responses in naïve mice. (FIG. 6A — FIG. 6D) WT mice were injected with PBS, PBS-treated RBCs, or CpG-treated RBCs via tail vein. Six hours following injection, mice were sacrificed. Quantification of (FIG. 6A) plasma interferon gamma *P<0.001, n=10-12 mice/group and (FIG. 6B) plasma IL-6 *P=0.036, n=7-16 mice/group, Kruskal-Wallis test. (FIG. 6C) H&E stained spleens sections revealing red pulp congestion and the presence of neutrophils (denoted by black triangles). (FIG. 6D) Quantification of spleen injury. 3-4 mice/group from 2 independent studies is shown, *P=0.02, multiple comparisons v control (pbs) Holm-Sidak test. (FIG. 6E) WT mice were intravenously injected with CpG-treated RBCs from WT or TLR9 KO mice. Quantification of plasma IL-6 is shown, n=6-7 mice/group, *P=0.035, t-test. (FIG. 6E — FIG. 6F) WT mice were intravenously injected with PBS- or CpG-treated RBCs. At 6 hr post-transfusion, spleens were harvested and subjected to RNA-seq analysis. (FIG. 6F) GO-term analysis and Heatmap (FIG. 6G) and of the top 25 differentially-expressed genes are shown.

FIG. 7A — FIG. 7F show RBC bound CpG is elevated during human sepsis. (FIG. 7A) RBC surface TLR9 expression is analyzed by flow cytometry and compared between healthy donors and patients with sepsis (n=15 healthy controls and 17 sepsis patients). *P=0.041, Mann Whitney U-test. Patients with P. falciparum-induced sepsis are denoted by dark circles. (FIG. 7B — FIG. 7C) Quantification of (FIG. 7B) 16 s DNA and (FIG. 7C) mitochondrial DNA on RBCs from healthy donors and patients with sepsis, as determined by quantitative PCR, are compared *P<0.001. (FIG. 7D — FIG. 7F) Comparison of (FIG. 7D) 16 s *P=0.037, (FIG. 7E) mitochondrial *P=0.045, and (FIG. 7F) combined DNA content *P=0.006 on RBCs from anemic patients with sepsis and non-anemic patients with sepsis. Each dot represents a different patient. Mann Whitney U-test for all comparisons.

FIG. 8A — FIG. 8E show RBCs from mice and chimpanzees express TLR9 on their surface. (FIG. 8A) MFI of surface TLR9 expression on unfixed, non-permeabilized RBCs from a chimpanzee (n=13 chimpanzees, one representative sample shown). (FIG. 8B) Surface TLR9 expression on RBCs from each chimpanzee (n=13). (FIG. 8C) Imaging of TLR9 expression on fixed and permeabilized chimpanzee RBCs. (FIG. 8D) Chimpanzee RBCs (250,000) were incubated with 100 nM or 1uM CpG-DNA. Dot plot and histogram demonstrating concentration dependent CpG binding by RBCs from one representative chimpanzee. (FIG. 8E) Concentration dependent CpG binding of RBCs from 10 chimpanzees, RBCs from 10 individual chimpanzees tested. *P=0.002, one-way ANOVA.

FIG. 9A — FIG. 9C show TLR9 and CD47 are in complex on the RBC surface. (FIG. 9A) Immunoprecipitation of TLR9 from RBCs reveals association with Band3 and CD47, IP, immunoprecipitation; IB, immunoblot. (FIG. 9B) Confocal imaging of CD47 and TLR9, Green, CD47 (2D3), Red, TLR9 (merged and Z stack images are shown in the bottom panel). (FIG. 9C) Imaging flow cytometry of RBCs labeled with TLR9 and CD47 antibodies following fluorescent CpG treatment. Gating strategy (left) and representative images (right) are shown.

FIG. 10A — FIG. 10C show CpG induces a conformational change in CD47 and malarial DNA leads to loss of CD47. (FIG. 10A) CpG induces a conformational change in CD47 on RBCs as denoted by increased detection of “damaged” RBCs by anti-CD47 2D3 antibody. Representative data from 1 out of 3 donors is shown. (FIG. 10B) CD47 detection on healthy RBCs following incubation with synthetic CpG corresponding to sequences from P. falciparum. (FIG. 10C) Summary statistics displaying loss of CD47 detection on RBCs from 7 healthy donors following incubation with P. falciparum CpG. *P=0.007 by t-test.

FIG. 11 shows a gating strategy on mouse splenocytes to identify red pulp macrophages. For analysis of red pulp macrophages (RPMs), splenocytes were labelled with the following antibodies (CD3, CD45 and F4/80). RPMs were identified live cells that were CD3⁻, CD45⁺, F4/80 high.

FIG. 12 lists the top differentially expressed genes in spleens from mice treated with RBCs or CpG-treated RBCs.

FIG. 13 shows the characteristics of the sepsis population as discussed in Example 2.

FIG. 14 shows a standard curve for K. pneumoniae generated using corresponding bacterial genomic DNA to quantify the DNA content in our studies.

FIG. 15 shows a standard curve for P. aeruginosa generated using corresponding bacterial genomic DNA to quantify the DNA content in our studies.

FIG. 16 shows a standard curve for S. pneumoniae generated using corresponding bacterial genomic DNA to quantify the DNA content in our studies.

FIGS. 17A-17D show plots of detected bacterial levels for the specified bacteria in donor RBC (sepsis patients).

FIG. 18 shows a plot of detected bacterial levels for S. aureus in donor RBC (sepsis patients) vs healthy controls.

FIG. 19 demonstrates that SARS-CoV-2 Spike protein is detectable on RBCs from patients with COVID. RBCs were obtained from a healthy donor or isolated from patient admitted to the MICU with COVID associated organ dysfunction. The RBC sample was obtained on the day of admission to the ICU. Immunofluorescence using antibodies reactive to the Spike protein (anti-S). Punctae are present on the RBCs of the COVID patient. Increased autofluorescence is also noted on COVID RBCs.

FIGS. 20A — 20D show that circulating RBCs demonstrate evidence of complement activation in patients with COVID 19. RBCs were obtained from healthy donors (HD) or critically ill patients with COVID 19. C3 (FIGS. 20A and 20B) and C4 (FIG. 20B) fragments are detected on RBCs from COVID patients but not healthy controls. C3 fragments increase from Day 0 to Day 7 of ICU admission days (FIG. 20C). These findings reflect complement activation in the COVID patients and demonstrate the use of RBCs as a real-time diagnostic measure for complement activation. FIG. 20D shows photomicrographs of RBC contacted with anti-compliment (anti-C3, anti-C4) or COVID spike protein (CoV-S) antibodies.

FIG. 21 are Beta Diversity Plots. 16S sequencing was performed on RBCs from healthy and septic patients. Principle Component Analysis (PCoA) of weighted UniFrac distances (abundance and type of bacterial species). The ellipses are 95% confidence intervals, differences in center of dispersion are tested by PERMANOVA, p=0.014. There are statistically significant differences between healthy and septic patient RBC-bound microbial DNA.

FIG. 22A demonstrates that 16S content on RBCs does not predict clinical outcomes. RBCs from septic patients were isolated and manually enumerated prior to DNA extraction and qPCR for 16S. 16S content on RBCs did not differ between culture negative and culture positive patients. 16S content on RBCs was similar between patients who survived to 30 days and those who died. Each circle represents an individual patient (n=70 patients).

FIG. 22B demonstrates bacterial DNA binds to RBCs. Bacterial DNA (1 ng or 10 ng) was incubated with 107 RBCs for 2 hours at 37° C. Following incubation, the RBCs were isolated from the supernatant by spinning over a 30% sucrose cushion. RBCs were manually counted and DNA was extracted. PCR for 16 s using probes specific for each pathogen was performed. Each color represents an individual healthy donor RBCs. We observed recovery of bacterial DNA from the RBCs.

FIGS. 23A and 23B demonstrate that pathogenic DNA can be detected on RBCs in culture negative patients. RBCs from patients with blood culture + or blood culture negative Staph Aureus infection (Staph Aureus Pneumonia) were isolated from. PCR for Staph Aureus DNA was performed on 5 uL plasma or 10⁷ RBCs (<5 uL RBCs). FIG. 23A demonstrates that Staph is detectable in the plasma of only culture positive patients, not culture negative. FIG. 23B is PCR of RBCs or plasma from two blood culture negative patients. Staph can be detected from the RBC preparation but not the plasma, confirming the hypothesis that RBCs are a reservoir for bacterial DNA. We do not detect pathogenic DNA on RBCs from healthy donors (data not shown).

FIG. 24 is a cartoon showing the proposed vascular endotype of ARDS, which is characterized by microthrombi, diffusion abnormalities, hypoxemia and preserved compliance. Endothelial cell death and thrombus/dysregulated coagulation are observed.

FIG. 25 demonstrates that human RBCs bind bacterial DNA. Bacteria-specific qPCR primers were used to quantify RBC-associated DNA following incubation of RBCs with genomic DNA. n=4 healthy donors. * P<0.05 vs 0 ng.

FIGS. 26A-26B demonstrate the presence of an RNA receptor (TRL7) on RBCs (a) FACs was performed on RBCs utilizing an antibody to the ectodomain of TLR7. (b) Imaging using the same antibody reveals surface staining (punctae) on RBCs. Band3 is an RBC membrane protein.

FIG. 26C demonstrates viral nucleic acid binding to RBCs (c) The right panel demonstrates RNA40 (sequence from HIV1) binding to RBCs. Each line represents an individual donor.

### DETAILED DESCRIPTION OF THE INVENTION

The requirement for culturing cells from a patient blood sample to diagnose a pathogenic infection is a serious hurdle to effective treatment for these conditions. Provided herein are compositions, kits, and methods to rapidly diagnose and treat pathogenic infections without the 3-day wait time of culture data.

Red blood cells (RBCs) comprise the majority of circulating cells in mammals and are essential for respiration. Although non-gas exchanging functions of the red cell such as chemokine regulation, complement binding and pathogen immobilization have been described, the immune function of RBCs remains enigmatic. RBCs transit through all tissues and are in contact with pathogen and self-derived inflammatory mediators in the circulation, positioning them as ideal messengers between remote organs. Indeed, it has recently been demonstrated that RBCs bind and scavenge nucleic acids away from the lung during basal conditions, yet it remains unknown whether RBC-nucleic acid binding contributes to the host immune response during inflammation.

Sepsis is the leading cause of death in U.S. hospitals, affecting over 1.6 million Americans every year. Current treatment for sepsis entails the administration of fluids and broad-spectrum antibiotics to patients. These measures in addition to supportive care are provided to patients to prevent the progression of sepsis to septic shock and multisystem organ failure, which both carry a high mortality and require intensive care. The antibiotics are administered to treat the underlying infection, while awaiting cell culture results to identify the pathogen. However, it usually takes at least three days for cell culture results to return, thus the lag between time of admission and time of treatment can be a delay administering proper therapy to the patient. Additionally, the use of broad-spectrum antibiotics without knowledge of the pathogen or appropriate tapering of antibiotics can lead to antibiotic resistance and the emergence of multi-drug resistant pathogens or “super-bugs”.

Respiratory infections are a major cause of death worldwide and pneumonia is the leading cause of sepsis, the deadly dysregulated host response to infection. Additionally, respiratory cultures are often not attainable and antibiotic prescribing decisions are made on empiric evidence, leading to the emergence of resistant infection.

COVID-19, the pandemic caused by the SARS-CoV-2 coronavirus can progress to pneumonia and Acute Respiratory Distress Syndrome (ARDS), resulting in an extraordinary level of ICU utilization and considerable mortality. Several pathophysiological features of COVID-19 associated ARDS are strikingly atypical, leading to the hypothesis that COVID-19 related ARDS represents a unique endotype of ARDS. There appear to be striking differences in the pulmonary physiology of COVID-19 patients compared to the features of ARDS precipitated by other respiratory pathogens and viruses (e.g. influenza). These observations suggest that dysfunction of the pulmonary vasculature contributes to the pathogenesis of COVID-19-induced ARDS and helped lead to the methods, compositions, and kits described herein.

Described herein are methods of detecting pathogenic infections. Our data demonstrate that RBCs are a rich source of pathogen-derived nucleic acids. Furthermore, using RBC-based nucleic acid amplification, we can detect pathogens prior to culture using less than a drop of blood. We demonstrate that RBCs can be used to detect respiratory pathogens in patients with pneumonia, sepsis and other ailments.

By the terms “patient” or “subject” as used herein is meant a mammalian animal, including a human, a veterinary or farm animal, a domestic animal or pet, and animals normally used for clinical research, including non-human primates, dogs and mice. More specifically, the subject of these methods is a human. In one embodiment, the subject is suspected of having a pathogenic infection, or a complication therefrom.

### Sample

All of the compositions, kits and methods described herein rely on the observation that RBC-containing samples can be used to detect, diagnose, treat and help predict outcome of various pathogenic infections. It is demonstrated that both human and murine red blood cells (RBCs) homeostatically bind CpG-containing DNA and sequester mtDNA released from dying cells. Under basal conditions, RBCs bound CpG-DNA, and it was established that under homeostatic conditions, the majority of cf-mtDNA (rich in CpG) was RBC-bound. Furthermore, data presented herein identified nucleic acids derived from bacteria bound to RBCs. In addition, it is demonstrated herein that RBC-containing samples are useful in detecting other pathogenic infections, including bacteria, viruses and parasites.

As used herein, the term “sample” or “patient sample” refers to a biological sample derived from a subject which contains red blood cells (RBCs). Also known as erythrocytes, RBCs are the most common type of cell found in the blood, with each cubic millimeter of blood containing 4-6 million cells.

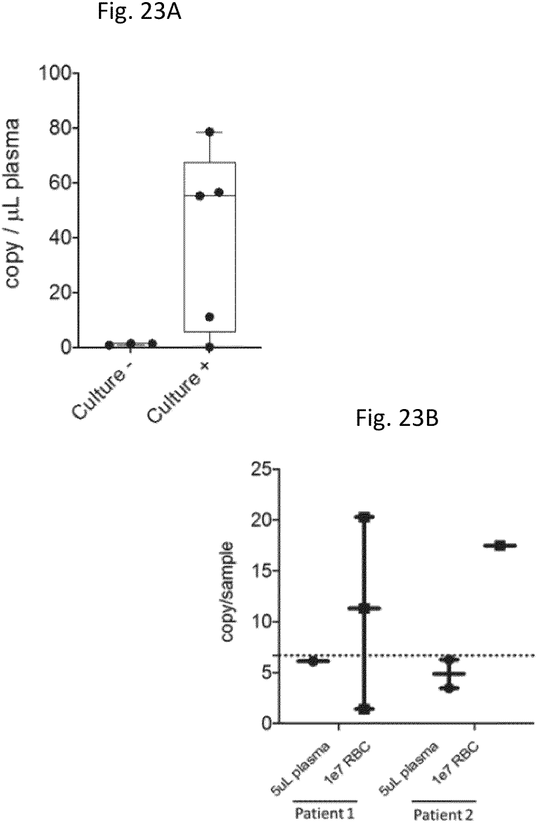

Significantly, the methods, compositions and kits described herein require only that the sample contain RBCs. There is no need for the sample to contain culturable bacteria or virus, as with the prior art methodologies for detecting infection (FIG. 23A and FIG. 23B). Rather, the RBCs are tested to determine the presence of certain markers derived from the pathogen(s), and thus provide critical information relating to the presence of a pathogenic infection, and the identification of the particular pathogen or pathogens causing the infection. Thus, in one embodiment, the sample is a blood sample containing RBCs from the subject which is free from culturable pathogen, e.g., bacteria, virus or parasite.

In certain embodiments, the sample is substantially free from all other blood components other than RBCs. Thus, in one embodiment, a sample of whole blood is obtained from a subject, and RBCs are isolated, concentrated or purified. In one embodiment, the sample is filtered to remove non-RBCs, and RBCs are isolated from the sample based on size. RBCs have a diameter of about 6-8 µM.

Another advantage of the compositions, methods, and kits described herein, is the small volume of sample required. In some embodiments, the sample is a drop of blood, which can be obtained from a finger stick at the point of care. Prior art methodologies require a venous blood draw, which is more invasive, more expensive, and requiring of specialized equipment and training. In contrast, in some embodiments, the compositions, methods and kits described herein require only about 1 µL to about 10 µL of blood, or a drop of blood or less. In one embodiment, the sample is about 1 µL, 2 µL, 3 µL, 4 µL, 5 µL, 6 µL, 7 µL, 8 µL, 9 µL, or 10 µL.In another embodiment, the sample is about 10 µL, 11 µL, 12 µL, 13 µL, 14 µL, 15 µL, 16 µL, 17 µL, 18 µL, 19 µL, or 20 µL. In yet another embodiment, the sample is about 10 µL or less. In another embodiment, the sample volume is about 1 µL to about 20 µL. In another embodiment, the sample volume is about 1 µL to about 10 µL. In another embodiment, the sample volume is about 2 µL to about 5 µL. Each of these ranges includes endpoints and all integers therebetween.

In another embodiment, the sample is less than about 1 mL. In another embodiment, the sample is about 100 µL, 200 µL, 300 µL, 400 µL, 500 µL, 600 µL, 700 µL, 800 µL, 900 µL, 1 mL, including all integers therebetween.

The sample must contain a sufficient number of red blood cells. In one embodiment, the sample contains at least 1 million RBCs. In another embodiment, the sample contains at least 1.5 million, 2 million, 2.5 million, 3 million, 3.5 million, 4 million, 4.5 million, 5 million, 5.5 million, 6 million, 6.5 million, 7 million, 7.5 million, 8 million, 8.5 million, 9 million, 9.5 million, or 10 million RBCs. In yet another embodiment, the sample contains at least 20 million, 30 million, 40 million, 50 million, 60 million, 70 million, 80 million, 90 million, or 100 million RBCs.

In some embodiments, a sample is obtained from a subject and treated to purify or enrich the sample for RBC. For example, the sample may be filtered to remove components smaller than and/or larger than a RBC, which is about 6-8 µM in diameter. The sample may also be sorted by density of the blood components, with the RBC component being isolated for use as described herein.

### Pathogens

The compositions, methods and kits described herein are, in some embodiments, used to detect and identify pathogenic infections. The pathogen may be a bacteria, virus, mycobacterium, parasite, fungi, or plasmodium. In some embodiments, more than one pathogen is present/detected.

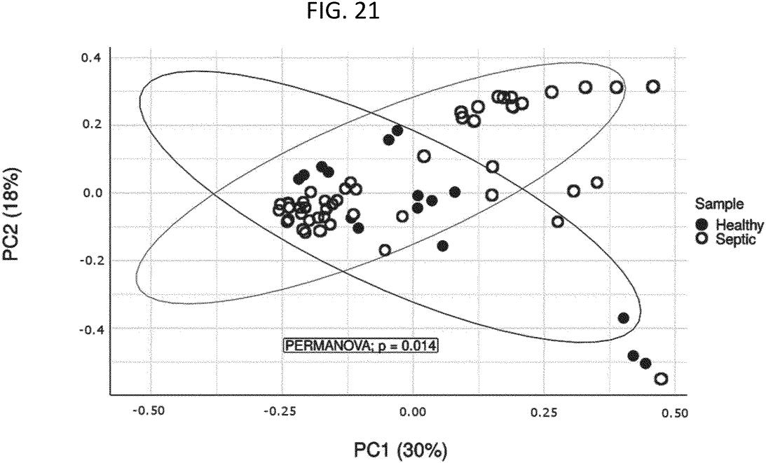

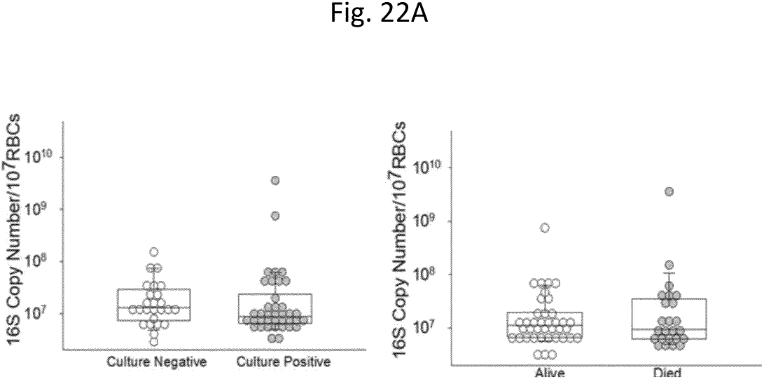

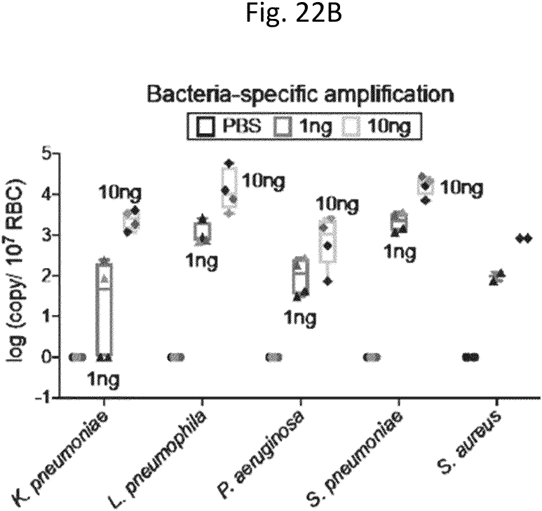

Evolutionarily conserved nucleic acid-sensing toll-like receptors (TLRs) identify nucleic acids derived from self and pathogens. They also play a central role in inflammation by promoting the secretion of inflammatory cytokines along with immune cell maturation and proliferation. It has recently been shown that RBCs express TLR9 and scavenge cell-free CpG-containing DNA under homeostatic conditions. Recent data demonstrate that TLR9 is conserved on mammalian RBCs and that RBCs bind bacterial and malarial DNA. Based on these observations, it is demonstrated that nucleic acid amplification of RBC-bound DNA enables pathogen detection prior to culture. It is demonstrated that we detect bacterial DNA binding to healthy naïve human RBCs using 16S PCR and probes specific for respiratory pathogens. As seen in FIG. 22B, we can recover bacterial DNA bound to RBCs without false positives, demonstrating that RBCs are useful as a diagnostic. We next asked whether we could amplify pathogen derived nucleic acid from RBCs collected from patients with pneumonia and negative blood cultures. As seen in FIG. 23, we can amplify pathogenic DNA from RBCs obtained from patients with culture negative pneumonia and sepsis. We further demonstrate herein that RBCs can be used to detect viral proteins, such as spike protein, and infection related complement. Collectively, these data demonstrate that RBCs can be utilized as a substrate for PCR-based diagnostics of infection.

It has previously been shown that RBCs express the pattern recognition receptor TLR9 and can bind and sequester CpG-containing DNA. This form of DNA is found in bacteria and viruses (Hotz AJRCCM 2018). As demonstrated herein, 16 s sequences present in bacteria are bound to RBCs. The compositions, methods and kits described herein, in some embodiments, utilize bacterial sequencing from nucleic acids bound to red blood cells, to reduce the wait time for targeted antibiotic therapy from a few days to a few hours. This innovation serves as a new tool for sepsis patients and to provide lifesaving therapy, as studies have shown that appropriate antibiotic administration early in sepsis improves mortality. A decrease in wait time for diagnostic results also prevents bacterial resistance from growing stronger with the usage of heavy antibiotics.

Thus, in one embodiment, the pathogen is a bacterium. The bacteria may be selected from Bacillus, Bartonella, Bordetella, Borrelia, Brucella, Campylobacter, Chlamydia, Chlamydophila, Clostridium, Corynebacterium, Enterococcus, Escherichia, Francisella, Haemophilus, Helicobacter, Legionella, Leptospira, Listeria, Mycobacterium, Mycoplasma, Neisseria, Pseudomonas, Rickettsia, Salmonella, Shigella, Staphylococcus, Streptococcus, Treponema, Ureaplasma, Vibrio, and Yersinia.

In another embodiment, the bacteria is Bacillus anthracis, Bacillus cereus, Bartonella henselae, Bartonella quintana, Bordetella pertussis, Borrelia burgdorferi, Borrelia garinii, Borrelia afzelii, Borrelia recurrentis, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtheriae, Enterococcus faecalis, Enterococcus faecium, Escherichia coli, Francisella tularensis, Haemophilus influenzae, Helicobacter pylori, Legionella pneumophila, Leptospira interrogans, Leptospira santarosai, Leptospira weilii, Leptospira noguchii, Listeria monocytogenes, Mycobacterium leprae, Mycobacterium tuberculosis, Mycobacterium ulcerans, Mycoplasma pneumoniae, Neisseria gonorrhoeae, Neisseria meningitidis, Pseudomonas aeruginosa, Rickettsia rickettsii, Salmonella typhi, Salmonella typhimurium, Shigella sonnei, Staphylococcus aureus, Staphylococcus epidermidis, Staphylococcus saprophyticus, Streptococcus agalactiae, Streptococcus pneumoniae, Streptococcus pyogenes, Treponema pallidum, Ureaplasma urealyticum, Vibrio cholerae, Yersinia pestis, Yersinia enterocolitica, and Yersinia pseudotuberculosis.

In one embodiment, the bacterium is S. aureus. In another embodiment, the bacterium is P. aeruginosa. In another embodiment, the bacterium is S. pneumoniae. In another embodiment, the bacterium is K. pneumoniae. In another embodiment, the bacterium is L. pneumophila. In another embodiment, the bacterium is E coli. In another embodiment, the bacterium is Legionella pneumophilia.

In some embodiments, the bacterium is one which is associated with pneumonia. In another embodiment, the bacterium is one which is associated with sepsis.

In another embodiment, the pathogen is a virus. In one embodiment, the virus is selected from Adenoviridae, Arenaviridae, Astroviridae, Bunyaviridae, Caliciviridae, Coronaviridae, Filoviridae, Flaviviridae, Hepadnaviridae, Hepeviridae, Herpesviridae, Orthomyxoviridae, Papillomaviridae, Paramyxoviridae, Parvoviridae, Picornaviridae, Polyomaviridae, Poxviridae, Reoviridae, Retroviridae, Rhabdoviridae, and Togaviridae.

In another embodiment, the virus is selected from Adenovirus, Lassa virus, Human astrovirus, Crimean-Congo hemorrhagic fever virus, Hantaan virus, Norwalk virus, Severe acute respiratory syndrome-related coronavirus, including Severe acute respiratory syndrome (SARS) virus, Severe acute respiratory syndrome coronavirus 2 (SARS-CoV2), Ebola virus, Marburg virus, Hepatitis C virus, yellow fever virus, dengue virus, West Nile virus, TBE virus, Hepatitis B virus, Hepatitis E virus, Herpes simplex, type 1, Herpes simplex, type 2, Varicella-zoster virus, Epstein-Barr virus, Human cytomegalovirus, Human herpesvirus, type 8, Influenza virus, Human papillomavirus, Measles virus, Mumps virus, Parainfluenza virus, Respiratory syncytial virus, Parvovirus B19, coxsackievirus, hepatitis A virus, poliovirus, rhinovirus, BK virus, JC virus, Smallpox, Rotavirus, Orbivirus, Coltivirus, Banna virus, Human immunodeficiency virus (HIV), Rabies virus, and Rubella virus. In one embodiment, the virus is SARS-CoV2 (the virus that causes COVID-19).

In another embodiment, the pathogen is a parasite. In one embodiment, the parasite is selected from Giardia lambia, Taxoplasma gondii, Trichomonas vaginalis, Entamoeba histolyticia, Plasmodium spp, Schistosoma mansoni, Tyypanosoma spp, and Leishmania spp.

In another embodiment, the parasite is one which is associated with malaria. In one embodiment, the parasite is Plasmodium falciparum, Plasmodium malariae, Plasmodium vivax, Plasmodium ovale, or Plasmodium knowlesi.

In another embodiment, the pathogen is a fungus. In one embodiment, the fungus is selected from Malassezia globosa, Trichophyton, Microsporum, or Epidermophyton type, Candida, Asperfillus, Cyrptococcums, Histoplasma, Pneumocystis, and Stachybotrys. Other bacteria, viruses, mycobacteria and parasites are known or may be discovered, and may be detected and/or identified using the compositions, methods and kits described herein.

### Methods of Detection

Provided herein are methods of detecting pathogenic infections. The methods include contacting a red blood cell-containing sample from a subject with a reagent capable of detecting a pathogen-associated molecule in the sample. In one embodiment, the subject is diagnosed with a pathogenic infection when the pathogen-associated molecule is detected in the sample.

In one embodiment, the reagent is able to detect the pathogen by forming a complex with a pathogen-associated molecule in the sample. In one embodiment, the subject is diagnosed with a pathogenic infection when the complex comprised of the pathogen-associated molecule and reagent is detected in the sample. In yet another embodiment, the reagent is capable of amplifying the pathogen-associated molecule. As used herein, the term “pathogen-associated molecule” refers to any biological molecule that is derived from, or indicates the presence of, a pathogen. Such molecules include nucleic acids (e.g., DNA, RNA, 16S rRNA, 18S rRNA, 28 s rRNA, mRNA, etc.) and proteins found in or on the pathogen, including surface proteins (e.g., glycoproteins, spike proteins, capsid proteins, F protein, G protein, etc.), antibodies elicited by the pathogen, and complement (e.g., C1, C2a, C4b, C3 (C3a, C3b), C5 (C5a, C5b), C6, C7, C8 and C9). As noted herein, in some embodiments of the invention, no culturable pathogen is present. However, the sample contains the pathogen-associated molecules, indicating the presence of the pathogen in the subject.

Various methods and techniques are known for detecting, amplifying or binding a pathogen-associated molecule in a sample. Such methods include nucleic acid-based methods (e.g., PCR-based methods) and protein-based methods (e.g., ELISA or flow cytometery). The reagents described herein are pathogen specific. By pathogen specific, it is meant that the reagent binds, identifies or amplifies a single, type, class, genus or species of pathogen or pathogen-associated molecule. For example, a pathogen specific reagent may be one that identifies total 16S rRNA found in bacteria. Alternatively, in a preferred embodiment, the pathogen specific reagent is one that identifies only a single genus or species of bacteria (e.g., a specific 16 s rRNA sequence, or specific spike protein). These examples are not meant to limit the term “pathogen specific” reagent, which is meant to encompass any reagent which can discriminate between two or more pathogens. In one embodiment, the reagent described herein comprises multiple reagents, where each reagent capable of detecting a different specific pathogen. In one embodiment, 2, 3, 4, 5, 6, 7, 8, 9, 10, 11, 12, 13, 14, 15, 16, 17, 18, 19, 20, 21, 22, 23, 24, 25, 30, 25, 40, 45, 50, 55, 60, 65, 70, 75, 80, 85, 90, 95, 100 or more different reagents are contained in the composition or utilized in the methods.

In one embodiment, the reagent is capable of detecting, binding, specifically complexing with, or measuring the level of a pathogen-associated molecule when present in the sample. In one embodiment, the reagents are those which are capable of detecting or measuring the amount or level of a pathogen using nucleic acids, e.g., DNA or RNA. In one embodiment, the pathogen is a bacterium and the reagent is capable of detecting or measuring the amount or level of 16S rRNA or DNA. See, e.g., Janda and Abbott, 16S rRNA Gene Sequencing for Bacterial Identification in the Diagnostic Laboratory: Pluses, Perils, and Pitfalls, J. Clin. Microbiol. September 2007 vol. 45 no. 9 2761-2764, which is incorporated herein by reference. Kits for performing the same are available commercially, including, without limitation, MicroSEQ® 500 16S rDNA Bacterial Identification System (Applied Biosystems). In one embodiment, the reagent targets the 16S V4 region.

16S ribosomal rRNA gene sequencing and reagents therefor are useful in some embodiments of the compositions and methods described herein. 16S rRNAs are about 1500 nucleotides in length, and vary based on the bacterial species in question, particularly in hypervariable regions. For 16S rRNA gene sequencing it is possible to utilize universal primers which bind to conserved regions, and thus, are able to amplify 16S rRNA gene from various bacterial species. Such bacteria are then elucidated using sequencing and 16S rRNA gene sequence databases, such as, but not limited to, Bacterial 16S Ribosomal RNA RefSeq Targeted Loci Project found at ncbi.nlm.nih.gov/refseq/targetedloci. Thus, in one embodiment, 16S rRNA gene sequencing is used. Primers useful in 16S rRNA sequencing are known in the art, and may be designed by the person of skill in the art. See, e.g., Klindworth et al, Nucleic Acids Res. 2013 Jan; 41(1): el, which is incorporated herein by reference. In one embodiment, 16 s rRNA is quantified using primer and probe having the following sequences: forward, 5′-AGAGTTTGATCCTGGCTCAG-3′ (SEQ ID NO: 5) and reverse, 5′-CTGCTGCCTYCCGTA-3′) (SEQ ID NO: 6), (probe, /56-FAM/TAA +CA+C ATG +CA+A GT+C GA/3BHQ_1/) (SEQ ID NO: 46). Other bacterial nucleic acids may be used. Suitable primers for amplifying the 16S rRNA region are provided below. These and other primers are known in the art. In one embodiment, the pathogen is a parasite and the reagent is capable of detecting or measuring the amount or level of 18S rRNA, circumsporozoite (CS) or K1-14 DNA. See, Moody, A., Rapid Diagnostic Tests for Malaria Parasites, Clin Microbiol Rev. 2002 Jan; 15(1): 66-78. In another embodiment, a fragment of the mitochondrial cytochrome c oxidase subunit III (coxIII) gene of P. falciparum is amplified, e.g., using the following primers (Forward: 5′-AGC GGT TAA CCT TTC TTT TTC CTT ACG- 3′ (SEQ ID NO: 1) Reverse: 5′-AGT GCA TCA TGT ATG ACA GCA TGT TTA CA-3′ (SEQ ID NO: 2).

In one embodiment, the pathogen is a virus and the reagent is capable of detecting or measuring the amount or level of a gene in the viral genome. In another embodiment, the reagent is capable of detecting or measuring the amount or level of ORF1b or N RNA or DNA. See, Chu et al, Molecular Diagnosis of a Novel Coronavirus (2019-nCoV) Causing an Outbreak of Pneumonia, Clinical Chemistry, Volume 66, Issue 4, April 2020, Pages 549-555 (epub Jan. 31, 2020). Such methods are effective for viruses such as SARS-CoV2. Other regions of the genome that may be detected include the capsid proteins including the hexon protein, the 5′ non-coding region, spike protein, etc.

In one embodiment, the pathogen is a mycobacterium and the reagent is capable of detecting or measuring the amount or level of a gene in the mycobacterial genome.

In one embodiment, the pathogen is a fungus and the reagent is capable of detecting or measuring the amount or level of a gene in the fungal genome. In another embodiment, the reagent is capable of detecting or measuring the amount or level of 28S rRNA. See, e.g., Sandhu et al. 1995. Molecular probes used for diagnosis of fungal infections. J. Clin. Microbiol. 33:2913-2919, which is incorporated herein by reference. Useful primers include SEQ ID NO: 48 [5′-GTG AAA TTG TTG AAA GGG AA-3′] and SEQ ID NO: 49 [5′-GAC TCC TTG GTC CGT GTT-3′]).

In other embodiments, the reagent is capable of binding DNA or RNA of a virus, fungi, mycobacterium or parasite.

The diagnostic reagent may be a polynucleotide or genomic probe that hybridizes to the pathogenic DNA or RNA. Such polynucleotides may be about 20, about 22, about 25 or more nucleotides in length. In another embodiment, the diagnostic reagent is a PCR primer-probe set that amplifies and detects a polynucleotide sequence of the subject bacteria. In one embodiment, the reagent is immobilized on a substrate. In another embodiment, the diagnostic reagent comprises a microarray, a microfluidics card, a computer-readable chip or chamber. Suitable assays utilizing the described polynucleotide, genomic probe, or a pair of PCR primers may include but are not limited to PCR, reverse-transcriptase PCR, quantitative PCR, southern blot analysis, dot-blot hybridization and fluorescence in situ hybridization (FISH). Conventional methods or tools can be utilized by one of skill in the art in designing suitable polynucleotide, genomic probe, or a pair of PCR primers as described, in view of the pathogen in question.

In other embodiments, the reagent is capable of detecting, binding, specifically complexing with, or measuring the level of a pathogen-associated protein when present in the sample. Such reagents and techniques are known in the art. In one embodiment, the pathogen-associated protein is detected via immunoassay. Suitable immunoassays include, without limitation, enzyme-linked immunosorbent assay (ELISA), flow cytometry, an immunohistochemical assay, a counter immuno-electrophoresis, a radioimmunoassay, radioimmunoprecipitation assay, a dot blot assay, an inhibition of competition assay, and a sandwich assay. In one embodiment, flow cytometry is utilized.

In another embodiment, one or more of the diagnostic reagents is labeled with a detectable label. In one embodiment, the label is an enzyme, a fluorochrome, a luminescent or chemi-luminescent material, or a radioactive material. In another embodiment, the diagnostic reagent is an antibody or fragment thereof specific for one of the subject biomarkers.

The measurement of the pathogen-associated molecule in the biological sample may employ any suitable ligand (reagent), e.g., antibody to detect the protein. Such antibodies may be presently extant in the art or presently used commercially, or may be developed by techniques now common in the field of immunology. As used herein, the term “antibody” refers to an intact immunoglobulin having two light and two heavy chains or any fragments thereof. Thus, a single isolated antibody or fragment may be a polyclonal antibody, a high affinity polyclonal antibody, a monoclonal antibody, a synthetic antibody, a recombinant antibody, a chimeric antibody, a humanized antibody, or a human antibody. The term “antibody fragment” refers to less than an intact antibody structure, including, without limitation, an isolated single antibody chain, a single chain Fv construct, a Fab construct, a light chain variable or complementarity determining region (CDR) sequence, etc. As used herein, the term “antibody” may also refer, where appropriate, to a mixture of different antibodies or antibody fragments that bind to the subject biomarker. Antibodies or fragments useful in the method of this invention may be generated synthetically or recombinantly, using conventional techniques or may be isolated and purified from plasma or further manipulated to increase the binding affinity thereof. It should be understood that any antibody, antibody fragment, or mixture thereof that binds to the pathogen-associated molecule as defined above may be employed in the methods of the present invention, regardless of how the antibody or mixture of antibodies was generated.

Similarly, the reagents may be tagged or labeled with reagents capable of providing a detectable signal, depending upon the assay format employed. Such labels are capable, alone or in concert with other compositions or compounds, of providing a detectable signal. Where more than one antibody is employed in a diagnostic method, e.g., such as in a sandwich ELISA, the labels are desirably interactive to produce a detectable signal. Most desirably, the label is detectable visually, e.g. colorimetrically. A variety of enzyme systems operate to reveal a colorimetric signal in an assay, e.g., glucose oxidase (which uses glucose as a substrate) releases peroxide as a product that in the presence of peroxidase and a hydrogen donor such as tetramethyl benzidine (TMB) produces an oxidized TMB that is seen as a blue color. Other examples include horseradish peroxidase (HRP) or alkaline phosphatase (AP), and hexokinase in conjunction with glucose-6-phosphate dehydrogenase that reacts with ATP, glucose, and NAD+ to yield, among other products, NADH that is detected as increased absorbance at 340 nm wavelength.

Other label systems that may be utilized in the methods of this invention are detectable by other means, e.g., colored latex microparticles (Bangs Laboratories, Indiana) in which a dye is embedded may be used in place of enzymes to provide a visual signal indicative of the presence of the resulting protein-antibody complex in applicable assays. Still other labels include fluorescent compounds, radioactive compounds or elements. Preferably, an antibody is associated with, or conjugated to a fluorescent detectable fluorochromes, e.g., fluorescein isothiocyanate (FITC), phycoerythrin (PE), allophycocyanin (APC), coriphosphine-O (CPO) or tandem dyes, PE-cyanin-5 (PC5), and PE-Texas Red (ECD). Commonly used fluorochromes include fluorescein isothiocyanate (FITC), phycoerythrin (PE), allophycocyanin (APC), and also include the tandem dyes, PE-cyanin-5 (PC5), PE-cyanin-7 (PC7), PE-cyanin-5.5, PE-Texas Red (ECD), rhodamine, PerCP, fluorescein isothiocyanate (FITC) and Alexa dyes. Combinations of such labels, such as Texas Red and rhodamine, FITC +PE, FITC + PECy5 and PE + PECy7, among others may be used depending upon assay method. Detectable labels for attachment to antibodies useful in methods described herein may be easily selected from among numerous compositions known and readily available to one skilled in the art of diagnostic assays.

In some embodiments of the methods described herein, the specific pathogen associated with the pathogen-associated molecule found in the sample, is identified. Thus, in some embodiments, DNA sequencing is performed. Methods for amplification and sequencing of the 16S rRNA gene in a sample are known in the art. See, e.g., A 16S rRNA gene sequencing and analysis protocol for the Illumina MiniSeq platform, Pichler et al, Microbiologyopen. 2018 Dec; 7(6): e00611, which is incorporated herein by reference. Briefly, the 16S rRNA gene is amplified using forward and reverse primers such as those described below.

| Primer name | Sequence | SEQ ID NO |
|---|---|---|
| 515f | 5′-GTGCCAGCMGCCGCGGTAA-3′ | 13 |
| 806r | 5′-TAATCTWTGGGVHCATCAGG-3′ | 14 |
| 27f | 5′-AGAGTTTGATCMTGGCTCAG-3′ | 15 |
| 1492r | 5′-GGTTACCTTGTTACGACTT-3′ | 16 |
| 27f* | 5′-GAGAGTTTGATCCTGGCTCAG-3′ | 17 |
| 1495r | 5′-CTACGGCTACCTTGTTACGA-3′ | 18 |
| 534R | ATTACCGCGGCTGCTGG | 19 |
| IlluminaF | CCTACGGGGNGGCWGCAG | 20 |
| IlluminaR | GACTACHVGGGTATCTAATCC | 21 |
| 515F (modified) | GTGCCAGCMGCCGCGGTAA | 22 |
| 515F (modified) | GTGYCAGCMGCCGCGGTAA | 23 |
| 806R | GGACTACHVGGGTWTCTAAT | 24 |
| 806R (modified) | GGACTACNVGGGTWTCTAA | 25 |
| 515F (modified) | GTGCCAGCMGCCGCGGTAA | 26 |
| 515F (modified) | GTGYCAGCMGCCGCGGTAA | 27 |
| 926R | GGACTACHVGGGTWTCTAAT | 28 |

In certain embodiments, compositions, kits and methods for detecting, diagnosing and treating COVID-19 patients are provided. It is demonstrated herein that viral RBC load correlates with disease severity and precedes evidence of an immune response; the onset of RBC-IgM/C3 correlates with evidence of the host inflammatory response and is followed by RBC-IgG during recovery; and the highest levels of RBC-IgM/C3 identifies patients at risk for progressive pulmonary insufficiency and TECs who are candidates for intervention with complement inhibitors.

### Complement

COVID-19, the pandemic caused by the SARS-CoV-2 coronavirus can progress to Acute Respiratory Distress Syndrome (ARDS), resulting in an extraordinary level of ICU utilization and considerable mortality. No medical therapies have been shown to improve mortality. Thus, it is imperative that research efforts focus on defining the underlying pathophysiology of COVID-ARDS as the basis for clinical intervention.

A potential mechanism of vascular injury contributing to ARDS in COVID-19 patients involves dysregulated complement activation. One of the oldest evolutionary defenses, the complement system serves as a first line defense against pathogens and is essential for the removal of dead cells and maintenance of organismal homeostasis. While the effector functions of opsonization, inflammation, chemotaxis and cytolysis promote pathogen clearance, dysregulated or excessive complement activation can lead to tissue injury and organ failure. Perhaps one of the best examples of this is the prothrombotic and anaphylotoxic effects of activated complement component 5 (C5a).

It is not immediately obvious why SARS-CoV-2 would activate complement more so than other etiologies of ARDS. Intriguingly, preliminary studies suggest a specific SARS-CoV-2 protein might directly interface with the complement system to increase the risk of thrombosis (T. Gao, et al, Highly pathogenic coronavirus N protein aggravates lung injury by MASP-2-mediated complement over-activation. medRxiv, 2020.2003.2029.20041962 (2020)). The nucleocapsid (N) protein of several coronaviruses, including SARS-CoV-2, was shown to bind directly and activate a key protease in the complement activation cascade, MASP-2, resulting in complement activation. In vivo administration of adenoviral vectors expressing SARS nucleocapsid strongly enhanced LPS-induced lung injury in mice, which was prevented by co-administration of MASP-2 neutralizing antibodies. In earlier, pre-clinical studies of SARS-CoV-induced lung injury, mice deficient in C3 were relatively protected from lung injury following SARS-CoV infection and exhibited less lung neutrophil recruitment and lower levels of cytokines in the lungs and circulation (Gralinski, et al, Complement Activation Contributes to Severe Acute Respiratory Syndrome Coronavirus Pathogenesis. mBio 9, (2018)). Further evidence for both alternative and classical pathway activation in COVID-ARDS is presented in recent autopsy studies where C4d and C5b-9 complex deposition was detected in the lung vasculature (Magro, et al, Complement associated microvascular injury and thrombosis in the pathogenesis of severe COVID-19 infection: A report of five cases. Translational Research, (2020)., Gao et al, Highly pathogenic coronavirus N protein aggravates lung injury by MASP-2-mediated complement over-activation. medRxiv, 2020.2003.2029.20041962 (2020)). Given the atypical risk factors for COVID-ARDS we argue that complement activation and wide-spread endothelial dysfunction contribute to the pathogenesis of COVID-ARDS.

We previously demonstrated that disruption of red cell scavenging function contributes to lung and systemic inflammation.⁶⁻⁸ RBCs bind complement through the highly expressed complement receptor CR1. Indeed, the majority of CR1 in the body is expressed on the nearly 33 trillion erythrocytes in the circulation. As described herein, RBCs from COVID-19 patients are analyzed for viral antigen and complement to provide a much-needed rapid diagnostic to identify patients with immune dysregulation who may benefit from complement inhibitory therapy.

### Methods

Provided herein, in one aspect is a method of diagnosing a pathogenic infection in a subject. The method includes contacting a red blood cell-containing sample (as described herein) from the subject with a reagent capable of detecting the pathogen in the sample and diagnosing the subject with a pathogenic infection when a pathogen-associated molecule is detected in the sample. In one embodiment, the sample is a blood sample containing RBCs from the subject which is free from culturable pathogen, e.g., bacteria, virus, mycobacterium or parasite. In another embodiment, the is substantially free from all other blood components other than RBCs. In one embodiment, the sample volume is about 1 µL to about 10 µL.

In some embodiments, DNA is extracted from the RBC containing sample. Methods for DNA extraction are known in the art. For example, the sample is centrifuged and the supernatant is discarded. A 20 mg / mL lysozyme solution (20 mM Tris-HC1, pH 8.0 / 2 mM EDTA, 1.2% Triton X-100) is added to the resulting precipitate at 37° C. Lysis treatment is performed for 30 minutes. Furthermore, a DNA extract can be obtained by performing column purification. This DNA extract may be used as a sample for PCR.

In some embodiments, the DNA is then subjected to amplification by PCR. Using the extracted DNA and primers, the 16S rRNA gene region (amplification target region) targeted for amplification is amplified by PCR, using primers known in the art, and as described herein.

Specifically, by using a primer set consisting of a forward primer and a reverse primer, e.g., the 16 s rRNA gene of a bacterium can be amplified. As the PCR reaction solution, for example, a nucleic acid synthesis substrate, a primer set, a nucleic acid synthase, a sample DNA, a buffer solution, and a solution containing water as the remaining components can be suitably used. Similar embodiments are contemplated for other genes of bacteria or other pathogens as described herein.

When the presence or absence of an amplification product by PCR is specified by a DNA chip, a label is added to the amplification product by PCR. The labeling method is not particularly limited, but a fluorescent label can be preferably used. When fluorescent labeling is performed by PCR, an amplification product in which only the ends are labeled can be generated using a fluorescently labeled primer. In addition, an amplification product containing a label therein can also be generated using a fluorescently labeled nucleic acid synthesis substrate. In any case, Cy5 or Cy3 can be suitably used as the fluorescent labeling component. Furthermore, as a label, it is also possible to use a label other than fluorescence, such as dicoxigenin, biotin, and a radioisotope.

Moreover, a general thermal cycler etc. can be used as an apparatus which performs PCR reaction. The reaction conditions for PCR can be performed, for example, as follows. (A) 94° C. 2 minutes, (b) 94° C. (DNA denaturation step) 30 seconds, (c) 60° C. (annealing step) 30 seconds, (d) 72° C. (DNA synthesis step) 60 seconds ((b) to (D) 35 cycles), (e) 72° C. 3 minutes. These conditions are provided as an example and not intended to limit the invention.

As a method for determining the presence or absence of bacteria, for example, electrophoresis can be performed. The electrophoresis can be performed by a general method such as agarose gel electrophoresis, acrylamide electrophoresis, or microchip electrophoresis. In electrophoresis, the presence or absence of bacteria is determined based on the size of the amplification product. When determining the presence or absence of target bacteria based on the size of the amplification product as in electrophoresis, if the size of the target gene region in multiple types of target bacteria is not so different, it is difficult to identify simultaneously in the system.

Therefore, it is preferable to use a DNA chip in order to specifically identify a plurality of types of bacteria simultaneously in one system. It is preferable to use a DNA chip on which a sequence complementary to a probe sequence is immobilized. These probes each have a specific sequence for each target bacterium (or other pathogen), and can hybridize only with the amplification product of the corresponding gene region, so that each target bacterium to be tested can be specifically detected simultaneously.

The DNA chip can be produced by an existing general method using the above probe. For example, when an affixed type DNA chip is produced, the probe can be immobilized on a glass substrate by a DNA spotter and a spot corresponding to each probe can be formed. When a synthetic DNA chip is produced, it can be produced by synthesizing a single-stranded oligo DNA having the above sequence on a glass substrate by a photolithography technique. Furthermore, the substrate is not limited to glass, and a plastic substrate, a silicon wafer, or the like can also be used. Further, the shape of the substrate is not limited to a flat plate shape, and may be various three-dimensional shapes, and a substrate having a functional group introduced so that a chemical reaction can be performed on the surface can be used.

The amplification product is dropped onto the DNA chip thus obtained, and the amplification product is hybridized to the probe immobilized on the DNA chip. And the kind of lactic acid bacteria to be examined can be specified by detecting the label of the hybridized amplification product.

In one embodiment, the pathogenic infection is a bacterial infection, and the reagent is capable of detecting bacterial DNA (CpG-containing DNA) in the sample. In one embodiment, the subject is diagnosed with a bacterial infection when bacterial DNA is detected in the sample. In another embodiment, the method includes treating the subject for the bacterial infection when diagnosed with the same. In one embodiment, the subject is culture-negative for bacterial infection. In another embodiment, the subject is for the bacterial infection when diagnosed with the same. The treatment may be an antibiotic that targets the pathogen. In one embodiment, the reagent is capable of detecting 16S ribosomal DNA, optionally the V4 region of 16S.

In one embodiment, the pathogenic infection is a viral infection, and wherein the reagent is capable of detecting viral DNA in the sample; and the subject is diagnosed with a viral infection when viral DNA is detected in the sample. In one embodiment, the subject is culture-negative for viral infection. In another embodiment, the method includes treating the subject for the viral infection when diagnosed with the same. In one embodiment, the treatment is an antiviral agent.

In another embodiment, the reagent is capable of detecting viral DNA, RNA, proteins or complement.

In one embodiment, the pathogenic infection is a parasitic infection, and wherein the reagent is capable of detecting parasite DNA in the sample; and the subject is diagnosed with a parasitic infection when parasite DNA is detected in the sample. In one embodiment, the subject is culture-negative for parasitic infection. In another embodiment, the subject is for the parasitic infection when diagnosed with the same. In one embodiment, treatment is an anti-malarial drug or a nitroimidazole.

In one embodiment, the pathogenic infection is a fungal infection, and wherein the reagent is capable of detecting fungal DNA in the sample; and the subject is diagnosed with a fungal infection when fungal DNA is detected in the sample. In one embodiment, the subject is culture-negative for fungal infection. In another embodiment, the subject is for the fungal infection when diagnosed with the same. In one embodiment, treatment is an antifungal drug such as an azole derivative.

In another embodiment, a method of detecting complement activation in a subject is provided. The method includes contacting a RBC containing sample with a reagent capable of identifying a complement protein, or fragment thereof. In one embodiment, the subject has, or is suspected of having, COVID19. In one embodiment, the method includes treating the subject with a complement inhibitor.

In another embodiment, a method of diagnosing a bacterial infection in a subject is provided. The method includes contacting a red blood cell-containing sample from the subject with reagents capable of detecting Staph aureus, Strep pneumoniae, Klebsiella pneumonia, Pseudomonas aeruginosa, E coli, and Legionella pneumophilia. In one embodiment, the subject has, or is suspected of having, pneumonia. Once the specific pathogen is identified, a pathogen-specific treatment may be administered to the subject.

### Treatment

In some embodiments of the methods described herein, the subject is treated for infection after being diagnosed with the same. Because the specific pathogen may be identified, treatment tailored to that pathogen may be started significantly earlier than if culture results are required.

In one embodiment, the treatment is an antibiotic that targets the pathogen. Suitable antibiotics include, without limitation, aminoglycosides such as amikacin, apramycin, arbekacin, bambermycin, butyrosine, dibekacin, dihydrostreptomycin, forthymicin, fradiomycin, gentamicin, ispamicin, kanamycin, micronomycin Neomycin, undecylenic acid neomycin, netilmicin, paromomycin, ribostamycin, sisomycin, spectinomycin, streptomycin, streptonicozid, and tobramycin; amphenicol, for example, azidamphenicol, chloramphenicol, chloram Ramphenicol palmirate, chloramphenicol pantothenate, florfenicol, and thianphenicol; Nsamycin, such as rifampin, rifabutin, rifapentine, and rifaximin; β-lactam, such as amidinocillin, amidinocillin, pivoxil, amoxicillin, ampicillin, aspoxillin, azidocillin, azulocillin, bacampicillin, benzylpenicillin, benzylpenicillin, benzylpenicillin, benzylpenicillin, Clometocillin, cloxacillin, cyclacillin, dicloxacillin, diphenicillin, epicillin, fenbenicillin, floxicillin, hetacillin, lenampicillin, mempicillin, methicillin, mezlocillin, nafcillin, oxacillin, pendademeta, Penicillin G benetamine, penicillin G Zatin, penicillin G benzhydrylamine, penicillin G calcium, penicillin G hydragamine, penicillin G potassium, penicillin G, procaine, penicillin N, penicillin O, penicillin V, penicillin V benzathine, penicillin V hydravamin, penimepicycline Pheneticillin, piperacillin, pivapicillin, propicillin, quinacillin, sulbenicillin, tarampicillin, temocillin and ticarcillin; carbapenems, such as imipenem; cephalosporins, such as 1-carba (dethia) cephalosporin, Cefadroxyl, cefamandole, cephatridine, cefazedone, cefazolin, cefixime, cefmenoxime, cefodizime, cefoniside, cefoperazone, cef Lanide, cefotaxime, cefotiam, cefpimizole, cefpirimide, cefpodoxime proxetil, cefloxazine, cefsulodin, ceftazidime, cefteram, ceftezol, ceftibutene, ceftizoxime, ceftriaxone, cefuroxime, cephazol Glycine, cephaloridine, cephalosporin, cephalothin, cefapirin sodium, cefradine, pibcephalexin (pivcefalexin), cephalotin, cefaclor, cefotetan, cefprozil, loracarbef, cefetamet, and cefepime; (cefetan), and cefoxitin; monobactams such as aztreo, Carmonam, and tigemonan; oxacephems such as flomoxef and moxolactam; lincosamides such as clindamycin and lincomycin; macrolides such as azithromycin, carbomycin, clarithromycin, erythromycin and derivatives, Josamycin, leucomycin, midecamycin, myomycin, oleandomycin, primycin, rokitamicin, rosaramicin, roxithromycin, spiramycin and troleandomycin; polypeptides such as amphomycin, bacitracin, capreo Mycin, colistin, enduracidin, enylomycin, fusafangin, gramicidin, gramicidin S, micamycin, polymycin Syn, polymyxin β-methanesulfonic acid, pristinamycin, ristocetin, teicoplanin, thiostrepton, tuberactinomycin, tyrosidin, tyrothricin, vancomycin, biomycin, virginiamycin, and zinc bacitracin; Cycyclin (spicycline), Chlortetracycline, Chromocycline, Demeclocycline, Doxycycline, Guamecycline, Limecycline, Meclocycline, Metacycline, Minocycline, Oxytetracycline, Penimecycline, Pipacycline, Ripacycline, Ripacycline Sancycline, senociclin, and tetracycline; 2,4-diaminopyrimidine, such as brodimoprim, Tetroxoprim and trimethoprim; nitrofurans such as furaltadone, furazolium, nifuraden, nifrater, nifluphorin, niflupyrinol, nifluprazine, niflutoinol, and nitrofurantoin; quinolones such as amifloxacin, sinoxacin, ciprofloxacin, difloxacin, enoxacin, floxacin, Flumequin, lomefloxacin, miloxacin, nalidixic acid, norfloxacin, ofloxacin, oxophosphate, pefloxacin, pipemidic acid, pyromido acid, roxoxacin, temafloxacin, and tosufloxacin; sulfonamides such as acetylsulfamethoxypyrazine, acetylsulfisoxazole, azosulfamide, Benzylsulfamide, chloramine-β, Roramin-T, dichloramine-T, hormones sulfathiazole, N2-formyl-Surufisomijin, N2-beta-D-gluco Sils Alpha sulfonylamide, mafenide, 4′-(methylsulfamoyl) sulfanyl anilides, p-Nitorosurufa Thiazole, nopril sulfamide, phthalyl sulfacetamide, phthalyl sulfathiazole, salazosulfadimidine, succinyl sulfathiazole, sulfabenzamide, sulfacetamide, sulfachlorpyridazine, sulfacryoidine, sulfacitin, sulfadiazine, sulf Phaziclamide, sulfadimethoxine, sulfadoxine, sulfaethidol, sulfaguanidine, sulfaguanol, sulfalene, sulfaloxic acid, sulfamerazine, sulfameter, sulfamethazi, Sulfamethizole, sulfamethomidine, sulfamethoxazole, sulfamethoxypyridazine, sulfamethol, sulfamidochrysidine, sulfamoxol, sulfanilamide, sulfanilamide methanesulfonic acid triethanolamine salt, 4-sulfanilamidosalicylic acid, N4-sulfanilylsulfanilamide, sulfanilylurea, N-sulfanilyl-3,4-xylamide, sulfanitran, sulfaperine, sulfaphenazole, sulfaproxyline, sulfapyrazine, sulfa Pyridine, sulfasomizole, sulfasimazine, sulfathiazole, sulfathiourea, sulfatramide, sulfisomidine, and sulfisoxazole; sulfones such as acedapsone, acediasulfone, Cetosulfone, dapsone, diathimosulfone, glucosulfone, solasulfone, succinsulfone, sulfanilic acid, p-sulfanilylbenzylamine, p, p′-sulfonyldianiline-N, N ‘digalactoside, sulfoxone, and thiazolesulfone; lipopeptides such as Oxazolidone, e.g., linezolid; e.g., ketolide, e.g., tethromycin; and various antibiotics, e.g., crotoctol, hexidine, magainin, methenamine, methenamine anhydromethylene-citric acid, methenamine, hippurate, mandel Included are methenamine acid, methenamine sulfosalicylate, nitroxoline, squalamine, xybornol, cycloserine, mupirocin, and tuberine.

In certain embodiments, it is advantageous to administer an anti-inflammatory composition. Suitable anti-inflammatory agents include, for example, steroidal anti-inflammatory agents, non-steroidal anti-inflammatory agents, or combinations thereof.

In some embodiments, antiviral agents are used. Useful antiviral agents include, but are not limited to, neuraminidase inhibitors, viral fusion inhibitors, protease inhibitors, DNA polymerase inhibitors, signal transduction inhibitors, reverse transcriptase inhibitors (such as nucleoside reverse transcriptase inhibitors and non-nucleoside reverse transcriptase inhibitors), interferons, nucleoside analogs, integrase inhibitors, thymidine kinase inhibitors, viral sugar or glycoprotein synthesis inhibitors, viral structural protein synthesis inhibitors, viral attachment and adsorption inhibitors, viral entry inhibitors (e.g., CCR5 inhibitors/antagonists) and their functional analogs. Neuraminidase inhibitors may include oseltamivir, zanamivir and peramivir. Viral fusion inhibitors may include cyclosporine, maraviroc, enfuviritide and docosanol. Protease inhibitors may include saquinavir, indinarvir, amprenavir, nelfinavir, ritonavir, tipranavir, atazanavir, darunavir, zanamivir and oseltamivir. DNA polymerase inhibitors may include idoxuridine, vidarabine, phosphonoacetic acid, trifluridine, acyclovir, foscarnet, ganciclovir, penciclovir, cidofovir, famciclovi, valaciclovir and valganciclovir. Signal transduction inhibitors include resveratrol and ribavirin. Nucleoside reverse transcriptase inhibitors (NRTIs) may include zidovudine (ZDV, AZT), lamivudine (3TC), stavudine (d4T), zalcitabine (ddC), didanosine (2′,3′-dideoxyinosine, ddl), abacavir (ABC), emirivine (FTC), tenofovir (TDF), delaviradine (DLV), fuzeon (T-20), indinavir (IDV), lopinavir (LPV), atazanavir, combivir (ZDV/3TC), kaletra (RTV/LPV), adefovir dipivoxil and trizivir (ZDV/3TC/ABC). Non-nucleoside reverse transcriptase inhibitors (NNRTIs) may include nevirapine, delavirdine, UC-781 (thiocarboxanilide), pyridinones, TIBO, calanolide A, capravirine and efavirenz. Viral entry inhibitors may include Fuzeon (T-20), NB-2, NB-64, T-649, T-1249, SCH-C, SCH-D, PRO 140, TAK 779, TAK-220, RANTES analogs, AK602, UK-427, 857, monoclonal antibodies against relevant receptors, cyanovirin-N, clyclodextrins, carregeenans, sulfated or sulfonated polymers, mandelic acid condensation polymers, AMD-3100, and functional analogs thereof. Antiviral agents also include immunoglobulins (antibodies) used in immunoglobulin therapy for the prevention of viral infection. Antiviral agents may also include, but are not limited to, the following: acemannan; alovudine; alvircept sudotox; aranotin; arildone; atevirdine mesylate; avridine, carbovir, cipamfylline; clevadine, crixivan, cytarabine; desciclovir; dideoxyinosine, dideoxycytidine, disoxaril, edoxudine; enfuvirtide, entecavir, enviradene; enviroxime; famciclovir; famotine; fiacitabine; fialuridine; floxuridine, fosarilate; fosfonet, gancyclovir, kethoxal; levovirin, lobucavir; lopinovir, memotine, methisazone; moroxydine, pirodavir, pleconaril, podophyllotoxin, rimantadine, sequanavir, somantadine, sorivudine, stallimycine, statolon; tilorone; tromantadine, valacyclovir, viramidine, viroxime, xenazoic acid, zalcitabine; zerit, zinviroxime, pyridine, oc-methyl- 1-adamantanemethylamine, hydroxy-ethoxymethylguanine, adamantanamine, 5-iodo-2′-deoxyuridine, trifluorothymidine, adenine arabinoside, 2′,3′-dideoxynucleosides such as 2′,3′-didoxycytidine, 2′,3′-dideoxyadenosine, 2′,3′-didoxyinosine, 2′,3′- didehydrothymidine, co-trimoxazole, 9-[2-(R)-[[bis[[(isopropoxy-carbonyl)oxy]-methoxy]phosphinoyl]methoxy]pro- pyl]adenine, (R)-9-[2-(phosphonomethoxy)- propyl] adenine, tenofivir disoproxil, TAT inhibitors such as 7-chloro-5-(2-pyrryl)-3H- 1,4-benzodiazepin-2(H)-one or nucleic acids that comprise one or more unmethylated CpG sequences essentially as disclosed in, e.g., U.S. Pat. No. 6,194,388.

In another embodiment, a complement inhibitor is administered. Complement inhibitors include, without limitation, OMS721, Eculizumab, Rauvlizumab, Coversin, CCX168, IFX 1, AMY-101, APL-2, ACH 4471, LNP023, Cemdisiran, C1INH, and LFG-316.

In another embodiment, an anti-malarial drug or a nitroimidazole are administered.

In one embodiment, the pathogenic infection is a COVID19 infection and a complement inhibitor is administered.

### Compositions and Kits

The compositions, kits and methods described herein include reagents which are capable of detecting, binding, specifically complexing with, or measuring the level of the pathogen-associated molecule. Such reagents include those which are capable of detecting, or measuring the abundance of, said molecule at the nucleic acid level. Suitable reagents include those for detection by polymerase chain reaction (PCR). Suitable reagents can be purchased commercially, e.g., such as those for the MagicPlex Sepsis system (Seegene), the VYOO rapid pathogen identification system (Analytik Jena Gmbh), PLEX-ID Pathogen Detector (Ibis Biosciences), and the SepsiTest (Molzym Molecular Diagnostics) for bacteria. Similar systems and reagents are known for viruses and parasites. In addition, suitable reagents may be designed by the person of skill in the art based on the published sequences of the specific pathogen of interest. In one embodiment, the reagents are PCR primers and/or probes. In addition, other suitable components are included to allow for the identification and/or quantitation of the subject pathogen. Such components include, e.g. enzymes, buffers and deoxynucleotides necessary for reverse transcription and/or PCR, preferably for qualitative and/or quantitative RT-PCR, detectable probes and/or an internal control. The present invention further provides a kit comprising the assay of the invention and optionally instructions for use.

The compositions, kits and methods described herein also, in some embodiments, include reagents which are capable of detecting, binding, specifically complexing with, or measuring the level of expression of the pathogen-associated molecule. Such reagents include those which are capable of detecting, or measuring the level of expression of, said pathogen-associated molecule at the polypeptide or protein level. In one embodiment, the reagents capable of detecting the biomarker(s) are proteins or polypeptides. In one embodiment, the proteins or polypeptides are antibodies or fragments thereof, e.g., such as those suitable for use in an ELISA or flow cytometry.

In one embodiment, at least one reagent is labeled with a detectable label. Suitable labels include, without limitation, an enzyme, a fluorochrome, a luminescent or chemi-luminescent material, or a radioactive material. In another embodiment, at least one reagent is immobilized on a substrate.

In one embodiment, the assay is an enzyme-linked immunosorbent assay (ELISA), and the reagents are thus, appropriate for that format. In another embodiment, the assay is flow cytometry. In another embodiment, the suitable assay is selected from the group consisting of an immunohistochemical assay, a counter immuno-electrophoresis, a radioimmunoassay, radioimmunoprecipitation assay, a dot blot assay, an inhibition of competition assay, and a sandwich assay. In another embodiment, the assay is one that utilizes electrochemiluminescent detection. In another embodiment, the diagnostic reagent is labeled with a detectable label. In one embodiment, the label is an enzyme, a fluorochrome, a luminescent or chemi-luminescent material, or a radioactive material.

Any combination of the described reagents for the detection of the subject pathogen-associated molecule can be assembled in a diagnostic kit for the purposes of the pathogenic infection. For example, one embodiment of a diagnostic kit includes reagents for 2, 3, 4, 5, 6, 7, 8, 9, 10, 11, 12, 13, 14, 15, 16, 17, 18, 19, 20, 21, 22, 23, 24, 25, 30, 25, 40, 45, 50, 55, 60, 65, 70, 75, 80, 85, 90, 95, 100 pathogens. In one embodiment, the kit includes reagents for 5 or 6 pathogens. In one embodiment, one or more of the reagents is associated or bound to a detectable label or bound to a substrate.

Provided herein is a kit which includes multiple reagents, each reagent capable of amplifying and/or identifying a different pathogen. For example, in one embodiment, a kit includes a set of primers for amplifying the 16s rRNA gene of 2, 3, 4, 5 or all 6 of Staph aureus, Strep pneumoniae, Klebsiella pneumonia, Pseudomonas aeruginosa, E. coli, and Legionella pneumophilia. In one embodiment, probes for detection of the amplicons are also provided.

For these reagents, the labels may be selected from among many known diagnostic labels, including those described above. Similarly, the substrates for immobilization may be any of the common substrates, glass, plastic, a microarray, a microfluidics card, a chip or a chamber.

It is intended that any of the compositions described herein can be a kit containing multiple reagents or one or more individual reagents. For example, one embodiment of a composition includes a substrate upon which one or more of the reagents are immobilized. In another embodiment, the composition is a kit also contains optional detectable labels, immobilization substrates, optional substrates for enzymatic labels, as well as other laboratory items. In one embodiment, the kit contains a standard for use as a control.

The invention is now described with reference to the following examples. These examples are provided for the purpose of illustration only and the invention should in no way be construed as being limited to these examples but rather should be construed to encompass any and all variations that become evident as a result of the teaching provided herein.

### EXAMPLES

### Example 1: Materials and Methods

### Sepsis Cohort

RBCs were obtained from patients enrolled in the Molecular Epidemiology of Severe Sepsis in the ICU cohort (MESSI) study at the University of Pennsylvania (42). Patients were eligible if they presented to the medical intensive care unit (MICU) with strongly suspected infection, at least 2 systemic inflammatory response syndrome (SIRS) criteria, and evidence of new end organ dysfunction in accordance with consensus definitions by the American College of Chest Physicians. Exclusion criteria included a lack of commitment to life sustaining measures, primary reason for ICU admission unrelated to sepsis, admission from a long-term acute care hospital, previous enrollment, or lack of informed consent. Human subjects or their proxies provided informed consent.

### RBC Isolation From Whole Blood

Whole blood was obtained from healthy volunteers in EDTA tubes and centrifuged for 10 minutes at 3,000 g. Plasma and buffy coat were removed. Packed RBC pellets were passed through a leukoreduction filter (Acrodisc White Blood Cell Syringe Filter, Pall Medical) with PBS or were isolated by magnetic-activated cell sorting with anti-Glycophorin A microbeads (CD235, Miltenyi Biotec). Filtered cells were centrifuged for 5 minutes at 800 g and supernatant was removed.

Specimens were also obtained from residual blood drawn for clinical purposes on admission to the ICU. Per clinical protocol, these samples were collected in EDTA-anticoagulated tubes, centrifuged at 3,000 g within 30 minutes for plasma analysis, and then stored at 4° C. RBCs were isolated within 24 hours of collection by magnetic-activated cell sorting as described above.

Chimpanzee blood samples (5-10 ml) were obtained from captive individuals (Pan troglodytes) housed at the New Iberia Research Center (Lafayette, Louisiana) in 10 ml ACD collection tubes (BD Biosciences). These samples were obtained for veterinary purposes only and represented leftover specimens from yearly health examinations. Whole blood was centrifuged at 1,500 g for 20 minutes (maximum acceleration and low brake speed). Buffy coats containing leukocytes were removed and red blood cells were resuspended in their respective plasma or PBS. Resuspended red blood cells were passed through a SepaCell R-500 II filter (Fenwal), an Acrodisc filter with Leukosorb media (PALL), or a high efficiency leukoreduction filter (Haemonetics) to remove remaining leukocytes. The red blood cells were stored in RPMI (Gibco).

### Flow Cytometry (Human)

For DNA binding studies, 250,000 RBCs were incubated with 100 nM or 1 uM CpG (10, 100 pmol / 250,000 cells). For studies involving DNA pre-treatments, one million RBCs were incubated with DNA at 37° C. for two hours with gentle shaking prior to antibody labelling. DNA treatments included the following: 400 nM, 4 µM CpG DNA (40, 400 pmol/106 cells, Synthesized by IDT), 20 µM synthetic CpG DNA representing sequences from P. falciparum (2,000 pmol / 106 cells), culture media from P. falciparum culture, or DNase (10 mg/ml, Sigma-Aldrich) treated culture media. DNase treated culture media was incubated with DNase at 37° C. for two hours with gentle shaking and then heated at 75° C. for 10 minutes. For TLR9 and CD47 studies, one million RBCs were labeled with the following antibodies: mouse monoclonal against TLR9 (5 µg, Clone 5G5, Abcam), CD47 (0.5 µg, CC2C6, Biolegend), or CD47 (1 µg, 2D3, eBioscience). For calcein studies, one million RBCs were first labeled with calcein- AM (5 uM, Fischer) prior to treatment with 8 uM of CpG DNA (800 pmol / 106 cells). For phosphatidylserine externalization studies, 250,000 RBCs were pre-treated with 100 nM or 1 uM CpG (10, 100 pmol / 250,000 cells) prior to labeling with Annexin V (Life Technologies) in Annexin V Binding Buffer (Life Technologies). FACS acquisition and analysis was performed using the LSR Fortessa (BD Biosciences) and FlowJo Software.

### Immunoprecipitation of TLR9 and CD47 From Human RBCs

Protein G beads (GE) were incubated with 5 µL of mouse monoclonal TLR9 antibody (26C593.2, Abcam) or CD47 antibody (2D3, eBioscience) and incubated for three hours at room temperature. RBCs (109) were lysed in RIPA buffer (50 mM Tris, 150 mM NaCl, 1% NP40, 0.1% SDS, 0.5% sodium deoxycholate and distilled H2O) for 15 minutes on ice. Lysed RBCs were added to the antibody and beads and incubated overnight at 4° C. Following centrifugation at 10,000 rpm for 20 minutes the beads were washed five times with RIPA buffer. 40 µl of sample buffer was added and then the beads were heated to 95° C. for 5 minutes. Proteins were resolved by SDS-page, and immunoblotting was performed with mouse monoclonal TLR9 (Abcam), rabbit monoclonal TLR9 (Abcam), mouse BAND3 (Santa Cruz), rabbit monoclonal CD47 (Abcam), or mouse CD47 (eBioscience).

### P. Falciparum Culture

P. falciparum cultures were propagated as previously described (43). Parasites were incubated with 4% packed red blood cells (BioIVT) in RPMI media with glutamine supplemented with 25 mM HEPES, 0.5 g/100 ml Albumax-II, 0.36 mM hypoxanthine, and 0.01 mg/ml gentamycin and cultured in an atmosphere of 90:5:5 N:02:CO2 gas. Culture media was spun for 5 minutes at 1,500 rpm prior to incubation with RBCs.

### Malarial DNA Binding by RBCs

RBCs (10⁶, 10⁷, 10⁸) isolated from healthy volunteers were incubated with 200 µl PBS or culture media from P. falciparum cultures at 37° C. for two hours with gentle shaking in DNA-Lo Bind tubes (Eppendorf). Following incubation, RBCs were added to 500 µl of 20% sucrose and centrifuged for 3 minutes at 13,000 rpm to separate the supernatant from the intact RBCs. DNA was extracted from samples using a commercially available kit (DNeasy, Qiagen). PCR amplification of a 500 bp fragment of the mitochondrial cytochrome c oxidase subunit III (coxIII) gene of P. falciparum was performed using the following primers (Forward: 5′-AGC GGT TAA CCT TTC TTT TTC CTT ACG- 3′ (SEQ ID NO: 1); Reverse: 5′-AGT GCA TCA TGT ATG ACA GCA TGT TTA CA-3′(SEQ ID NO: 2)). Amplicons were verified by gel-purification and sequencing by the University of Pennsylvania Genomics Analysis Core.

### Legionella DNA Binding by RBCs

RBCs isolated from healthy volunteers were incubated with 0, 0.1, 1, or 10 ng of Legionella DNA at 37° C. for two hours with gentle shaking. After incubation, RBCs were washed and DNA was extracted from the RBC pellet. Presence of 16 s bacterial DNA was quantified by qPCR as described below. Legionella DNA was a gift from Dr. Sunny Shin at the University of Pennsylvania.

### Electron Microscopy

RBCs (10⁵) isolated from healthy volunteers were treated with 100 nM Class B, 2006 CpG DNA (10 pmol/10⁵ cells) at 37° C. for two hours with gentle shaking. Following incubation, cells were washed with PBS and fixed with 0.05% glutaraldehyde (Polysciences). Scanning Electron Microscopy sample preparation and acquisition was performed by the University of Pennsylvania Electron Microscopy Resource Laboratory. Cells were examined with a JEOL 1010 electron microscope fitted with a Hamamatsu digital camera and AMT Advantage image capture software.

### Confocal Microscopy

RBCs (10⁵) isolated from healthy volunteers were incubated with 100 nM Class B, 2006 CpG DNA (10 pmol/10⁵ cells) or 2336 GpC control DNA (10 pmol/105 cells) at 37° C. for two hours with gentle shaking. For F-actin, Spectrin, and Band3 staining, RBCs were then fixed using 0.05% glutaraldehyde, permeabilized with 0.1% Triton-X, washed, and stained (F-actin: 5 µg/ml, ab130935, Abcam; Spectrin: 1:100 dilution, ab2808, Abcam; Band3: 10 µg/ml, sc133190, Santa Cruz). For TLR9 surface staining, RBCs were first labeled with the membrane dye PKH (MIDI26-1KT, Sigma) and then washed and fixed with 0.05% EM grade glutaraldehyde prior to the addition of TLR9 antibody (20 µg/ml, Clone 5G5, Abcam). For intracellular staining of chimpanzee RBCs, RBCs were fixed, permeabilized, washed, and stained with mouse anti-TLR9 (5 µg/ml, 26C593.2, Novus Biologicals) in addition to PKH dye. For CD47-TLR9 colocalization staining, RBCs were fixed, permeabilized, washed, and stained with mouse CD47-2D3 (10 µg/ml, EBioscience) and rabbit TLR9 (10 µg/ml, ab 187148, Abcam). Confocal images were acquired using the SCTR Leica Confocal microscope, and images were analyzed using ImageJ software.

### Imaging Flow Cytometry

For TLR9 staining and CpG binding, RBCs (5×10⁵) isolated from healthy volunteers were incubated with varying doses — 1 µM (100 pmol / 5×10⁵ cells), 500 nM (50 pmol/5×10⁵ cells), 100 nM (10 pmol / 5×10⁵ cells), 50 nM (5 pmol / 5×10⁵ cells), 25 nM (2.5 pmol / 5×10⁵ cells), 10 nM (1 pmol / 5×10⁵cells), or 5 nM (0.5 pmol / 5×10⁵ cells) — of Class B, 2006 CpG DNA AlexaFluor-594 (IDT) at 37° C. for two hours with gentle shaking. RBCs were then labeled with FITC-conjugated mouse monoclonal TLR9 (1 µg, clone 5G5, Abcam).

For TLR9-CD47 double staining and CpG binding, RBCS (1e6) were incubated with 400 nM Class B, 2006 CpG DNA AlexaFluor-674 (40 pmol/106 cells) (synthesized by IDT) at 37° C. for two hours. RBCs were then labeled with FITC-conjugated mouse monoclonal TLR9 (1 µg, clone 5G5, Abcam) and Pacific Blue-conjugated mouse monoclonal CD47 (1 µg, clone CC2C6, Biolegend). TLR9 and CD47 expression and CpG binding to RBCs were visualized and quantified by Image Stream analysis. Raw Image Files (RIF) were analyzed in IDEAS software. The automated feature finder generated Fisher Discriminant values to differentiate sub populations of RBCs. Mean pixel and Intensity features clearly discriminated two populations of cells that exhibited different morphologies by phase contrast. The intensity feature is a measure of the fluorescence intensity of the whole RBC, while the mean pixel feature indicates the mean pixels per RBC. These subpopulations were denoted as either ‘smooth’ or ‘altered.’ Imaging flow cytometry was performed by the Penn Flow Cytometry and Cell Sorting Core.

### Osmotic Fragility Assay and Hemolysis Assays

5×10⁵ freshly isolated RBCs from human donors were incubated with indicated concentrations of CpG in PBS for two hours at 37° C. with gentle shaking at 90 rpm. Cells were pelleted and washed in 1 mL PBS and resuspended in 100 uL PBS, followed by incubation in 15 mL NaCl solutions (0.9%, 0.6%, 0.3%, 0.1%, or 0% NaCl) in deionized water at room temperature for 10 minutes. The reactions were centrifuged at 800 g for 10 min at 4° C. on slow brake. Hemoglobin content in supernatant was determined using QuantiChromTM Hemoglobin Assay according to manufacturer’s protocol. Hemoglobin content in the reaction of RBCs without CpG treatment and incubated with water (0.00 Osml/L) was set at 100% and data were normalized against it.

### Detection of mtDNA and 16 s in Human Samples

DNA was extracted from 10⁷ RBCs using a commercially available kit (DNeasy, Qiagen). mtDNA in samples was quantified using SYBR green I (Roche) by qPCR assay (Life Technologies). Primer sequence for human COXI is as follows: (forward, 5′-TGATCTGCTGCAGTGCTCTGA-3′ (SEQ ID NO: 3) and reverse, 5′-TCAGGCCACCTACGGTGAA-3′(SEQ ID NO: 4)). 16 s in samples was also quantified by qPCR assay (Life Technologies) using TaqmanTM Fast Universal PCR Master Mix (Applied Biosystems) and the following primer sequence and probe: (forward, 5′-AGAGTTTGATCCTGGCTCAG-3′ (SEQ ID NO: 5) and reverse, 5′-CTGCTGCCTYCCGTA-3′) (SEQ ID NO: 6), (probe, /56-FAM/TAA +CA+C ATG +CA+A GT+C GA/3BHQ_1/).

### Experimental Animals

C57B⅙ animals were purchased from the Charles River Laboratories Inc. TLR9 knockout mice were produced by S. Akira and provided by Edward Behrens (Children’s Hospital of Philadelphia). All experimental procedures were performed on 8-12-week-old mice, 20-25 g in weight. Animal studies were conducted in accordance with the Institutional Animal Care and Use Committee at the University of Pennsylvania.

### RBC Isolation From Murine Whole Blood

Whole blood from mice was collected via intracardiac puncture and RBCs were isolated as previously described (14). Briefly, blood was centrifuged for 10 minutes at 1,500 g, the plasma and buffy coat were removed, and packed RBC pellet was passed through a leukoreduction filter (Acrodisc White Blood Cell Syringe Filter, Pall Medical) with PBS. Filtered cells were centrifuged for 5 minutes at 800 g and supernatant was removed.

### Flow Cytometry (Mouse)

RBCs were isolated as described above and 106 RBCs were labeled with monoclonal TLR9 (5 µg, Clone 5G5, Abcam). In separate studies RBCs (106) were incubated with 400 nM or 4 µM 1826 CpG DNA (40 or 400 pmol / 1e6 cells) (IDT) at 37° C. for two hours with gentle shaking. RBCs were then labeled with PE CD47 (1 µg, clone miap301, Biolegend). FACS acquisition and analysis was performed using the LSR Fortessa (BD Biosciences) and FlowJo Software.

### Murine CpG-RBC Transfusion Model

RBCs (10⁹) were incubated with 150 µM 1826 CpG DNA (15,000 pmol / 10⁹ cells, synthesized by IDT) at 37° C. for two hours with gentle shaking. Following incubation and subsequent washes, PBS, RBCs, or CpG-treated RBCs were injected via tail vein. Six hours following injection, mice were sacrificed with intraperitoneal injections of ketamine/xylazine (80/10 mg/kg). Whole blood was obtained via cardiac puncture and centrifuged for 5 minutes at 10,000 rpm to isolate plasma. Interferon gamma and IL-6 in the plasma was measured by ELISA (R&D). Spleens were formalin fixed prior to paraffin embedding. H&E staining was performed by the Penn Veterinary School Comparative Pathology Core. Spleen H&Es were read and scored by a veterinary pathologist. Spleen injury was determined by the presence of neutrophils or red pulp congestion (0 for absent 1 for present). 3-4 mice/group from 2 independent studies is shown. RNA-sequence analysis was performed on the spleen of mice transfused with PBS-RBCs or CpG-RBCs (Genewiz). Data analysis was performed using DEseq2 (Genewiz) and EdgeR.

To confirm the role of CpG binding by TLR9 on RBCs, WT or Tlr9-KO RBCs were transfused along with 50 ug of 1826 CpG-DNA into WT mice. Six hours following injection, mice were sacrificed as previously described, and IL-6 in the plasma was measured by ELISA (R&D).

### Erythrophagocytosis Studies

GFP-positive RBCs obtained from GFP-expressing mice (C57BL/6-Tg(UBC-GFP)30Scha/J from Jackson Laboratories) were incubated with 50 µg CpG at 37° C. for two hours with gentle shaking in DNA- Lo Bind tubes (Eppendorf). Following incubation and subsequent washes, 200 µl GFP RBCs and DNA-treated GFP RBCs were transfused to C57BL/6 mice through retro orbital injection (45 minutes before transfusion, 200 µl of blood was removed from each mouse). Drops of whole blood were collected through tail snips at 0 min, 5, 20, 40 and 60 min for flow cytometry detection of GFP positive cells in the circulation. One hour following injection, mice were sacrificed with isoflurane and cervical dislocation. Spleens were harvested, weighed, minced, and passed through a 70 µm nylon mesh to obtain a single-cell suspension. Cells were counted, mixed with ACK Lysis Buffer (Thermo Fisher Scientific), and centrifuged at 400 g for 6 minutes to isolate white blood cells. Cells (106 cells) were resuspended in 100 µl staining buffer (PBS+0.1% sodium azide) and incubated with anti-mouse CD16/32 antibody (Fc block, eBiosciences, San Diego, CA) for 10 min at 4° C. to block nonspecific binding. Cells were then stained with the following antibodies (F4/80 PE, #123109, Biolegend; CD11b eFluo450, M1/70, #101211, Biolegend; CD45 APC, clone 30-F11, #559864, BD Pharmingen; CD3 BUV395, 145 2C11, #564661, BD) or appropriate isotype controls (0.25-1.5 µg/ 106 cells) for 30 minutes at 4° C. Cells were then spun and resuspended in staining buffer for viability staining (Fixable Viability Dye, eFluor780, #65-0865-14, eBioscience) for 30 minutes at 4° C. Cells were fixed in 2% paraformaldehyde for 10 minutes, spun and resuspended in 500 µl PBS. At least 50,000 CD45+ cells were analyzed with an LSR Fortessa (BD Biosciences). Gating analysis was performed using FlowJo software (FlowJo, LLC, Ashland, Oregon).

### Clearance of CpG-treated RBCs

Whole blood was obtained from C57Bl/6J or Tlr9-KO mice via cardiac puncture and centrifuged for 5 minutes at 700 g. The plasma was removed, and the remaining cell pellet was leukoreduced to isolate RBCs. WT and Tlr9-KO RBCs were labeled with PKH26 or PKH67 respectively (Sigma). Labeled WT and Tlr9-KO RBCs were incubated together with 1826 CpG DNA (synthesized by IDT) at a concentration or 25 nM for two hours at RT. Following incubation, cells were centrifuged and resuspended in PBS. Each mouse received 2×108 erythrocytes. Blood was sampled from the tail vein at specified time points (0, 5, 10, 20, 40 and 60 minutes) following transfusion. The percentage of WT v KO RBCs in whole blood at each specified timepoint was compared.

### Measurement of mtDNA on Murine Plasma and RBCs Following Infection

Whole blood from mice infected with Legionella pneumophila, influenza virus A/H1N1/ PR/8, or Toxoplasma gondii were obtained from collaborators at the University of Pennsylvania (Shin Lab, x Lab, and Hunter Lab respectively). Methods for each individual infection are previously described (References). Whole blood was centrifuged for 5 minutes at 10,000 rpm. 5 ul aliquots of plasma and RBCs were obtained for DNA extraction and mtDNA quantification as described below.

### Measurement of mtDNA in Murine Plasma and RBCs Following Cecal Ligation and Puncture.

Cecal ligation and puncture was performed on mice as previously described (Reference Dimitra’s Temple Paper). Mice were anesthetized with ketamine/xylazine (80/10 mg/kg) prior to performing a 1-2 cm midline laparotomy that exposed the cecum and adjoining intestines. The cecum was ligated at its base, 1 cm below the ileo-cecal valve, and was then punctured twice with an 18-gauge needle. The cecum was then gently squeezed to extrude cecal matter from the perforations. The skin was then closed after returning the cecum to the peritoneal cavity. To resuscitate mice, 1 ml of pre-warmed 0.9% saline solution was injected subcutaneously. Sham procedures included laparotomies without ligation and puncture. 6 hours after procedures, mice were sacrificed with ketamine/xylazine (80/10 mg/kg). Whole blood was obtained via cardiac puncture and centrifuged for 5 minutes at 10,000 rpm. 5 ul aliquots of plasma and RBCs were obtained for DNA extraction and mtDNA quantification as described below.

### Detection of mtDNA on Murine RBCs and Plasma

Murine mtDNA in plasma and RBCs were measured as previously described (ref). mtDNA in samples was quantified using SYBR green I (Roche) by qPCR assay (Life Technologies). Primer sequences for murine COXI are as follows: (forward, 5′-GCCCCAGATATAGCATTCCC-3′ (SEQ ID NO: 7) and reverse, 5′-GTTCATCCTGTTCCTGCTCC-3′) (SEQ ID NO: 8).

### Oligodeoxynucleotides (ODNS) Utilized

2006 CpG (human): 5′T*C*G*T*C*G*T*T*T*T*G*T*C*G*T*T*T*T*G*T*C*G*T*T-
3′ (SEQ ID NO: 9)

1826 CpG (mouse): 5′-T*C*C*A*T*G*A*C*G*T*T*C*C*T*G*A*C*G*T*T-3′ (SEQ ID
NO: 10)

Malarial CpG 2 (18): 5′- T*C*G*T*C*G*T*C*G*T*C*G*T*C*G*T*C -3′ (SEQ ID NO:
11)

Control GpC (human): 5′G*G*G*GAGCAGCTGCTGG*G*G*G*G*G-3′ (SEQ ID NO:
12)

*phosphorothioate bond

### Statistics

Differences between groups were compared using a t-test, Mann-Whitney U test, or one way ANOVA, as appropriate. All statistical analyses were performed using Sigma Plot 13 software (Systat Software Inc). A p<0.05 was considered significant for all analyses.

### Example 2: Erythrocyte Nucleic Acid Sensing Through TLR9 Regulates Red Cell Survival and Immune Responses

### Mammalian Erythrocytes Express Surface TLR9 and Bind Pathogen DNA

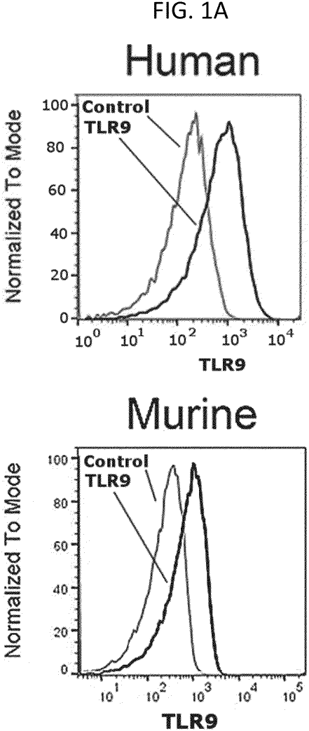

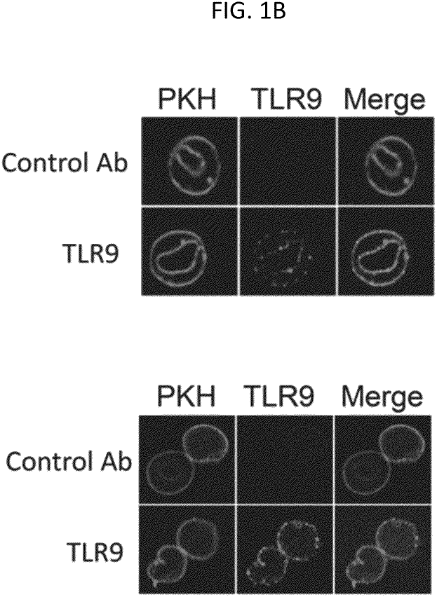

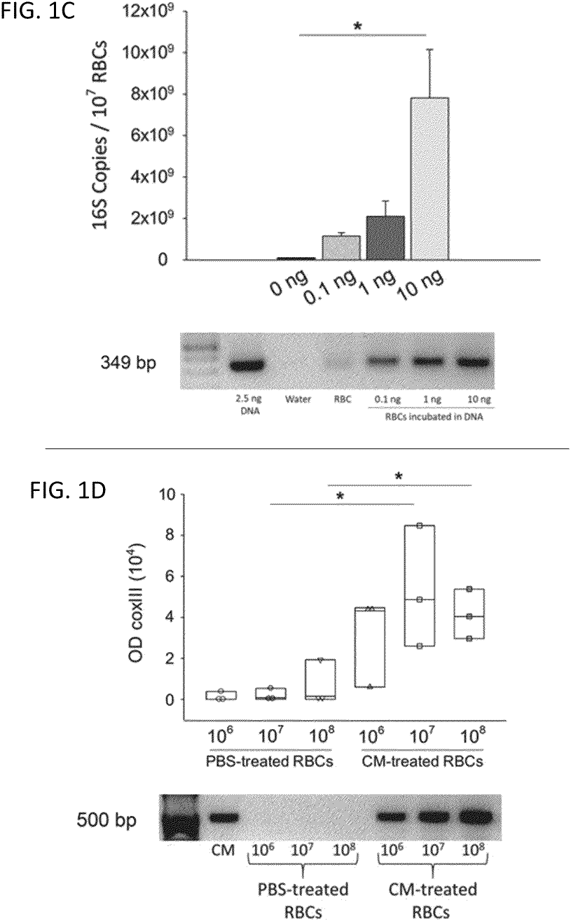

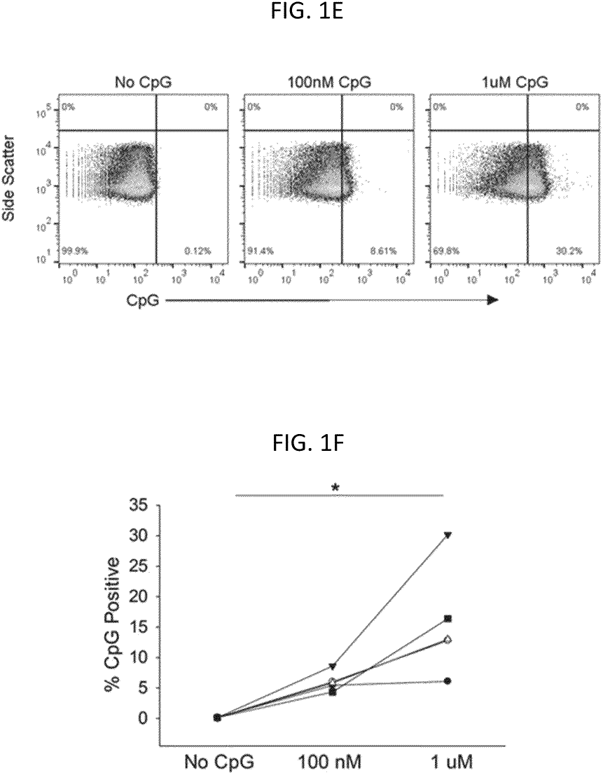

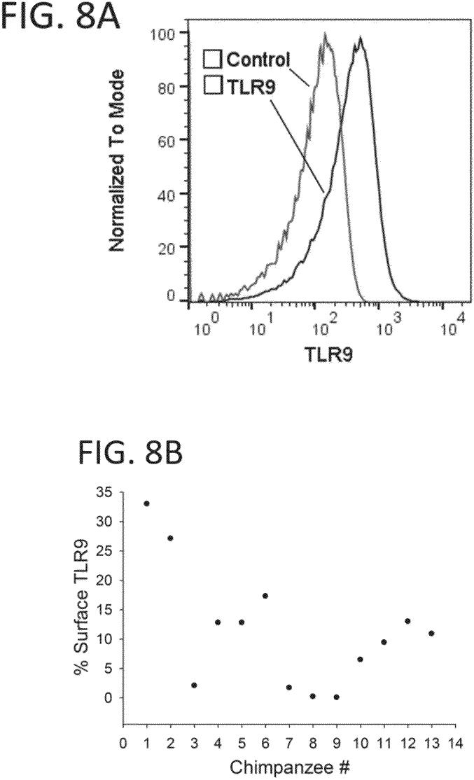

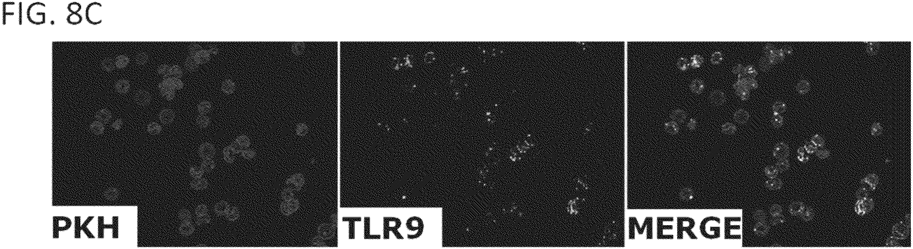

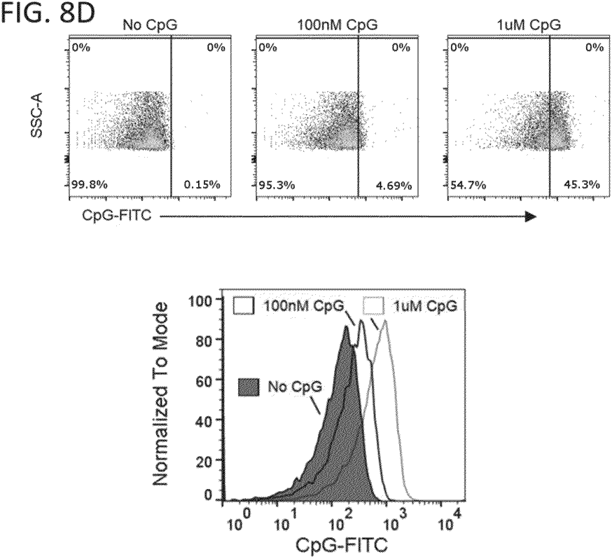

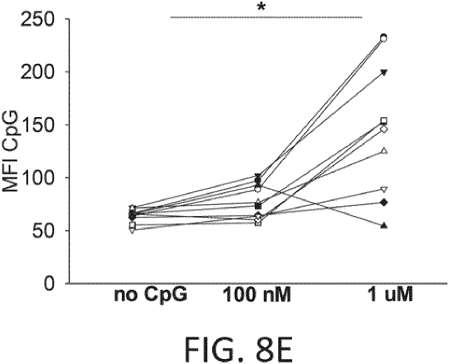

Recent studies have identified the presence of TLR9 on the surface of intestinal epithelial cells, splenic dendritic cells, and a minor fraction of peripheral blood mononuclear cells (PBMCs) (15-17). We have previously detected intracellular TLR9 in RBCs, but were unable to detect surface expression of TLR9.(14) We asked whether TLR9 might also be expressed on the surface of RBCs. Using antibodies to the ectodomain of TLR9, TLR9 was detected on intact non-permeabilized human and murine RBCs (FIG. 1a). We verified RBC TLR9 expression using confocal microscopy (FIG. 1b). We next asked whether RBCs from healthy human donors would bind bacterial DNA or malarial mtDNA by incubating RBCs with genomic DNA from Legionella pneumophilia or media from P. falciparum erythrocyte culture. Following incubation with bacterial DNA or P. falciparum DNA, RBCs were isolated and PCR for 16 s (bacterial DNA) or coxIII (malarial mtDNA) was performed. We found a dose dependent increase in amplifiable microbial DNA on RBCs following incubation with bacterial or malarial DNA (FIGS. 1c and d). We confirmed the ability of human RBCs to bind malarial DNA using synthetic CpG based on sequences found in the P. falciparum genome (FIGS. 1e and f)(18). We next asked whether RBCs from non-human primates express surface TLR9 and bind CpG. Chimpanzee RBCs, like their human counterparts, express surface TLR9 and bind CpG (FIG. 8). These data indicate that TLR9 is expressed on the surface of RBCs, where it can bind cell-free DNA, and that this function is conserved in our closest ape relative.

### DNA Binding Results in Altered RBC Structure and Function

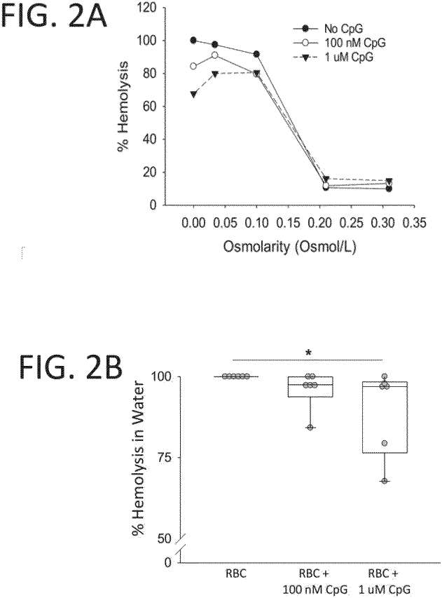

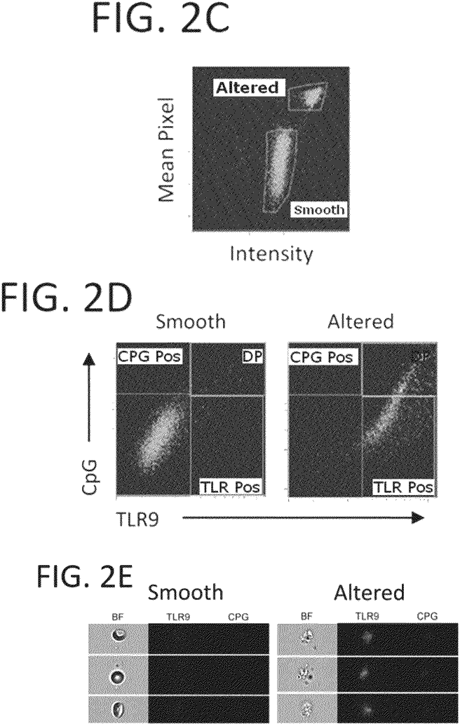

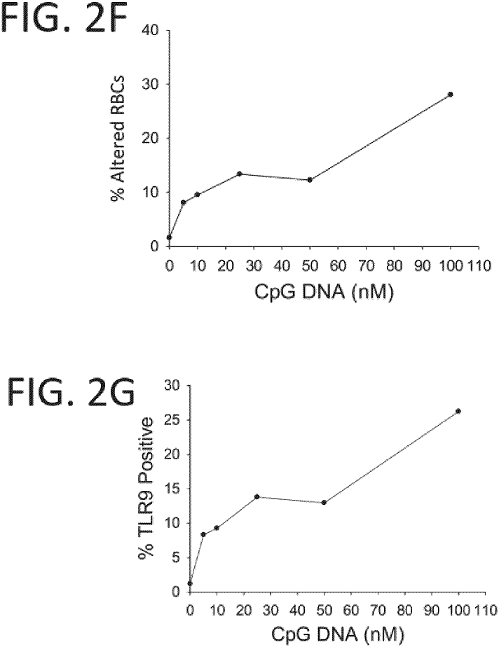

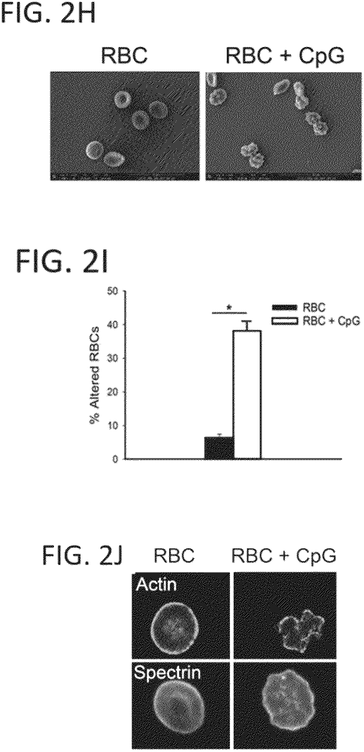

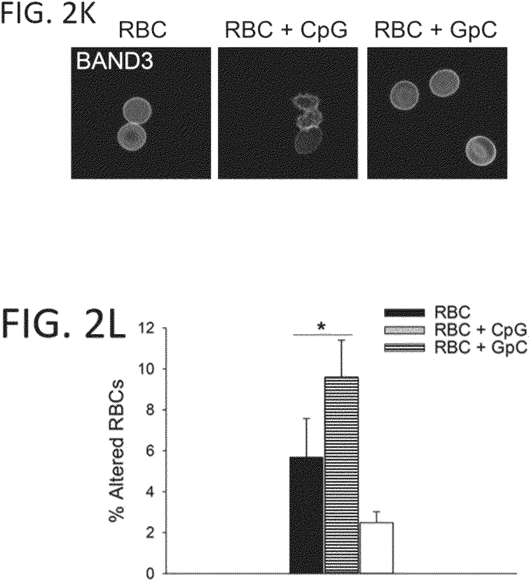

We previously found that under homeostatic conditions, the majority of cell-free mtDNA was sequestered on RBCs, with very little cell-free mtDNA detectable in the plasma in both mice and humans (14). However, during both sterile inflammation and infection, cell-free CpG-containing mtDNA is elevated in the plasma (8, 10). We therefore examined the effect of surplus cell-free CpG on RBC function by measuring osmotic fragility of RBCs. As shown in FIGS. 2a and b, the addition of CpG resulted in reduced osmotic fragility. We next examined the morphology of RBCs following CpG incubation using imaging flow cytometry. This analysis identified a population of RBC with aberrant morphology following incubation with CpG. The automated feature finder was utilized to discriminate subpopulations of RBCs based on morphology, and Mean Pixel and Intensity features were identified to best differentiate smooth from altered cells (FIG. 2c), the latter of which were more TLR9 and CpG positive (FIG. 2d); representative images of smooth and altered cells are shown in FIG. 2e. In the presence of low levels of extracellular CpG DNA, RBCs remained morphologically unaltered. However, addition of increasing amounts of CpG (50-100 nM/0.5 million RBCs) resulted in malformed RBCs that were also TLR9 positive (FIGS. 2f and g). Extensive alterations of RBC morphology following high dose CpG treatment (100 nM) were confirmed by electron microscopy (FIGS. 2h and i), which revealed an association between RBC shape changes and marked differences in the distribution of the cytoskeletal proteins spectrin and actin following CpG binding (FIG. 2j). We have previously shown that TLR9 is associated with the integral membrane and ion transport protein Band 3 in RBCs (14). Since CpG DNA is known to lead to a conformational change in the TLR9 ectodomain (19), we examined the distribution of Band 3 following CpG DNA treatment. As a control, we used GpC DNA, which binds TLR9 without causing conformational changes (19). As shown in FIGS. 2k and l, treatment with CpG DNA, but not GpC DNA, led to alterations in Band 3 distribution. Taken together, these data indicate that CpG DNA binding alters RBC morphology in a TLR9 dependent manner.

### CpG Binding by RBCs Leads to Loss of CD47

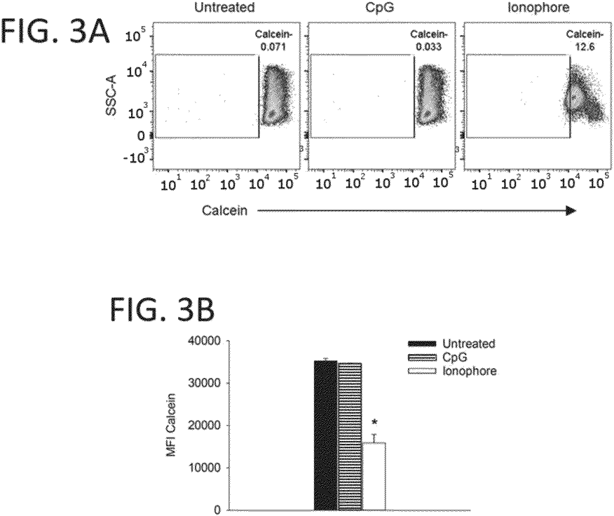

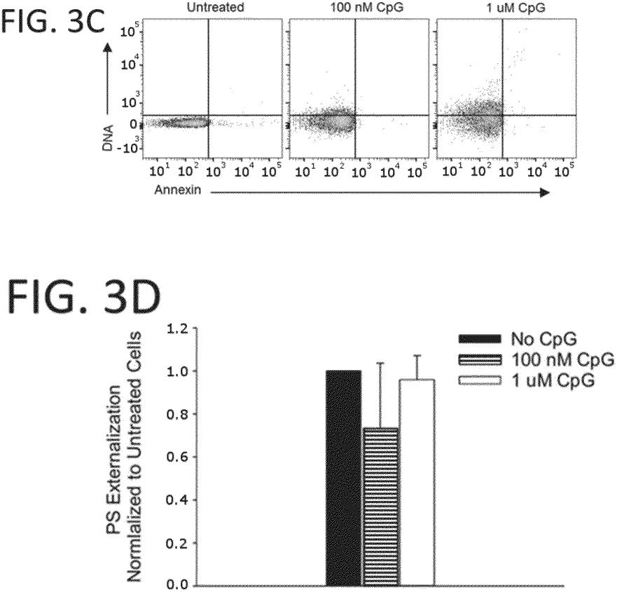

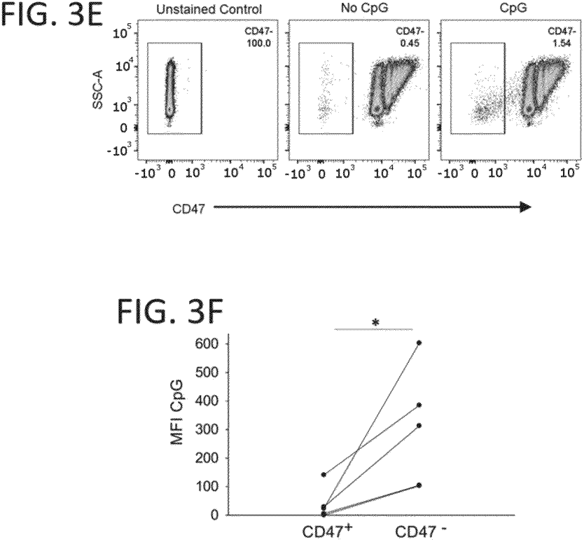

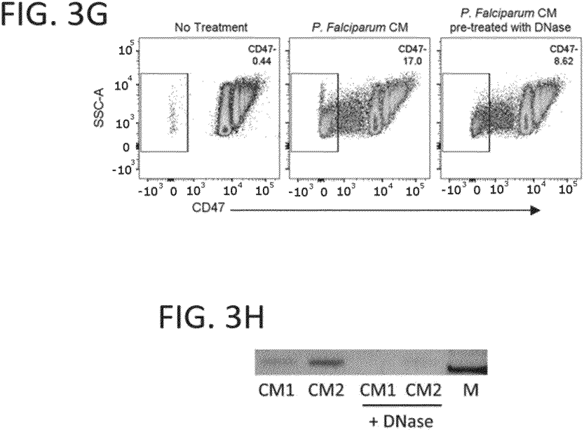

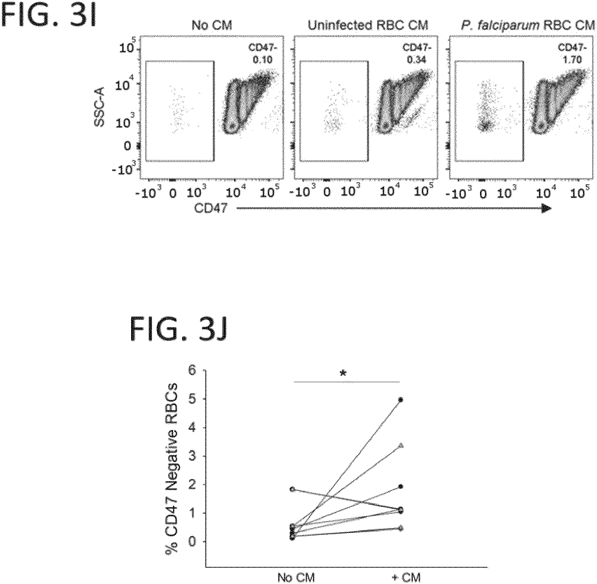

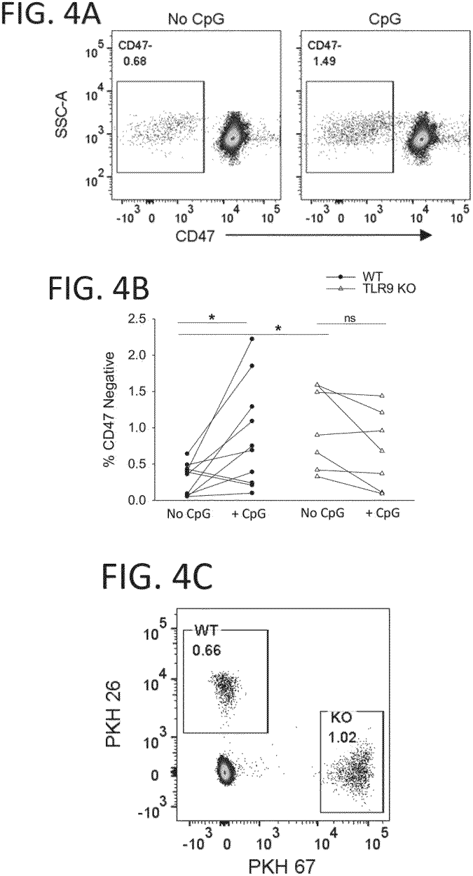

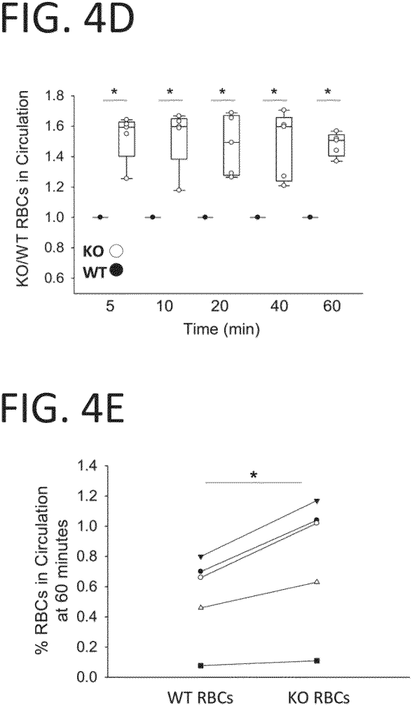

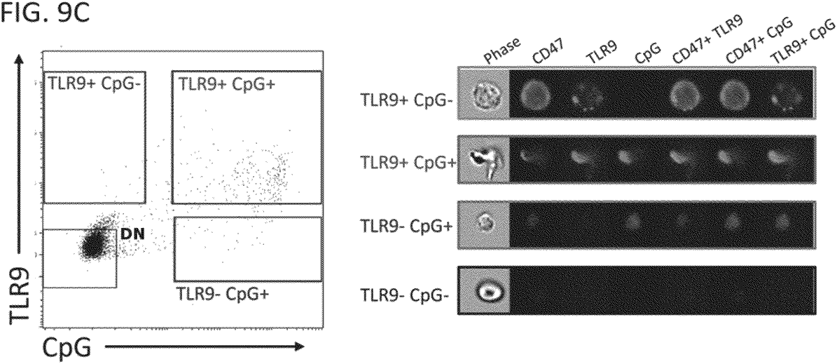

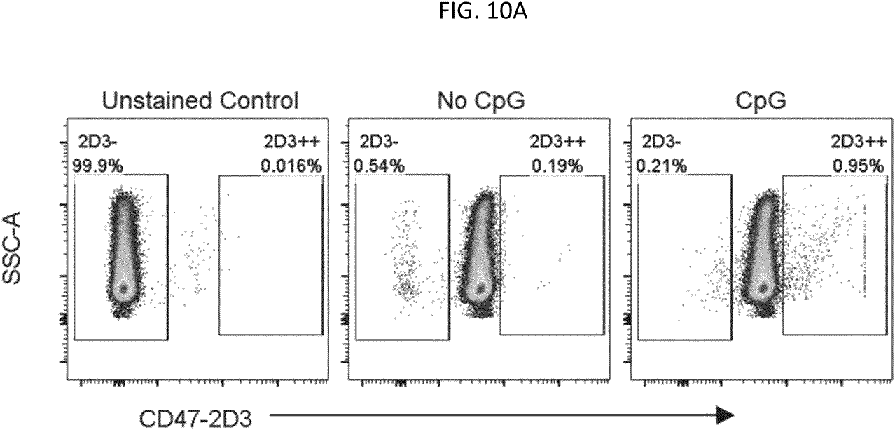

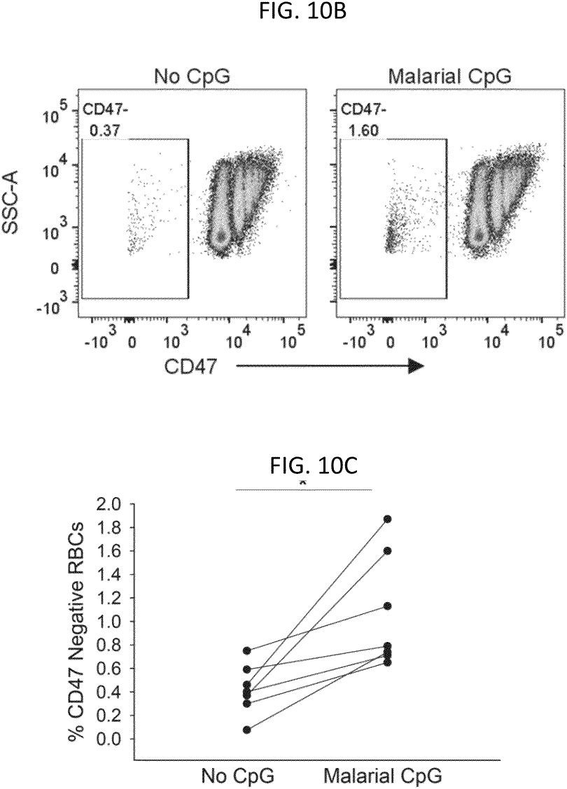

Because we observed extensive morphologic alterations of the RBC following CpG binding we next asked whether DNA binding would alter RBC viability. RBCs were incubated with CpG prior to assessment of viability by loss of membrane integrity as indicated by loss of the dye calcein-AM. As seen in FIGS. 10A and 10B, CpG did not lead to a loss of viability. While multiple factors determine RBC survival, PS externalization serves as an “eat me” signal and CD47 expression serves as a marker of self or “don’t eat me” signal. We next examined both PS externalization and CD47 expression following CpG treatment of RBCs. While we did not observe an increase in PS positive cells, we did observe loss of CD47 as measured by binding of the antibody CC2C6 which detects the antiphagocytic epitope of CD47 (FIGS. 3C-E). We observed that following the addition of CpG DNA, cells that were CC2C6 negative bound higher amounts of DNA than those that were CC2C6 positive (FIG. 3F).

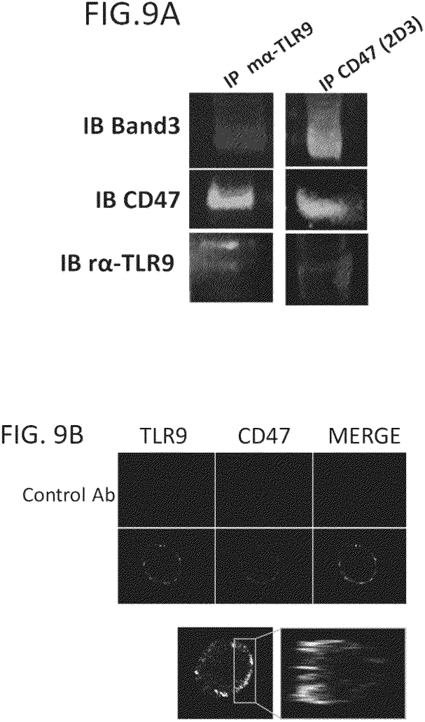

CD47 is in association with the Band 3 complex, a macrocomplex of proteins in the RBC membrane (20, 21). Using proximity ligation assay and co-immunoprecipitation, we previously observed an association between TLR9 and Band 3 in RBCs (14). We therefore asked whether CD47 was also complexed with TLR9. Using TLR9 and CD47 antibodies to probe RBC lysates, we found that these two proteins co-immunoprecipitated (FIG. 9a). We confirmed this physical interaction using confocal microscopy, which revealed co-localization of TLR9 and CD47 in the RBC membrane (FIG. 9b). Because we had observed morphological alterations in the RBC membrane following CpG binding, we performed imaging flow cytometry to better characterize the distribution of surface TLR9 and CD47 in intact RBCs following CpG DNA addition. We incubated RBCs with 100 nM CpG and found that CpG DNA led to alterations in RBC structure as well as CD47 and TLR9 redistribution (FIG. 9c). TLR9 positive, CpG negative cells demonstrated punctate surface TLR9 staining and uniform surface CD47 distribution, whereas TLR9 positive, CpG positive cells showed alteration of the membrane and clustering of TLR9 and CD47 (FIG. 9c). These findings suggest that a conformational change of CD47 occurs following CpG-binding by RBCs.

We next asked whether conformational changes of CD47 were associated with the altered localization observed after CpG binding. Conformational changes in CD47 can be detected by an increase in binding of the anti-CD47 antibody 2D3. (22) This antibody detects an epitope on CD47 that has undergone conformational changes and is present “damaged” experimentally aged and sickle RBCs (22, 23). Incubation of RBCs with CpG for 2 hours led to increased detection of this CD47 epitope using the 2D3 antibody (FIG. 10a).

We next asked if malarial DNA would induce loss of CD47 in uninfected erythrocytes from healthy human donors. The loss of CD47 detection was dependent on nucleic acids present in the culture medium since DNase treatment diminished this loss (FIGS. 3g and h). Incubation of naïve human RBCs with P. falciparum culture supernatants led to the appearance of a CD47 negative erythrocyte population in all but one experiment (FIGS. 3i and j). We confirmed these findings using synthetic CpG sequences, based on sequences found in the P. falciparum genome, which also led to a loss of CD47 detection on RBCs obtained from healthy donors (FIGS. 10b&c) (18).

### CpG-treatment of Murine RBCs Leads to Loss of CD47

The antibody mIAP301 blocks the antiphagocytic CD47 epitope on murine erythrocytes (24). We thus asked whether CpG-treatment would lead to a loss of CD47 detection using this antibody. CpG-treatment of erythrocytes from wildtype (WT), but not TLR9 knock-out (KO) mice, resulted in a TLR9-dependent loss of CD47 detection (FIGS. 3a and b). Interestingly, TLR9 KO mice also exhibited a higher number of CD47 negative RBCs in the circulation. These findings may reflect decreased clearance of CD47 negative RBCs, since recent studies have demonstrated a role for monocyte/macrophage TLR9 signaling in erythrophagocytosis (11-13).

To determine the effect of RBC TLR9 mediated CpG-binding on RBC clearance, we treated WT and TLR9 negative RBCs with CpG, and infused these CpG-treated RBCs back into naïve WT mice. Examining the relative proportion of WT and TLR9 negative RBCs in circulation, we found that following ex vivo CpG-treatment, TLR9-negative RBCs remain in the circulation longer than WT RBCs (FIGS. 3c-e).

### RBC-Bound CpG Is Elevated During Infection and Sterile Inflammation

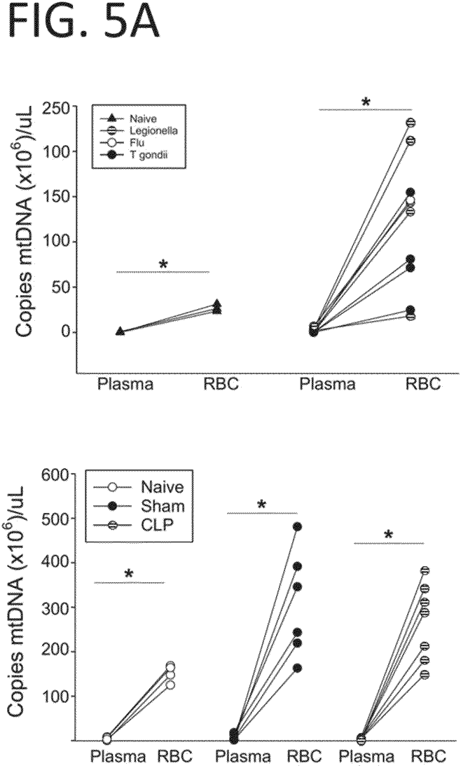

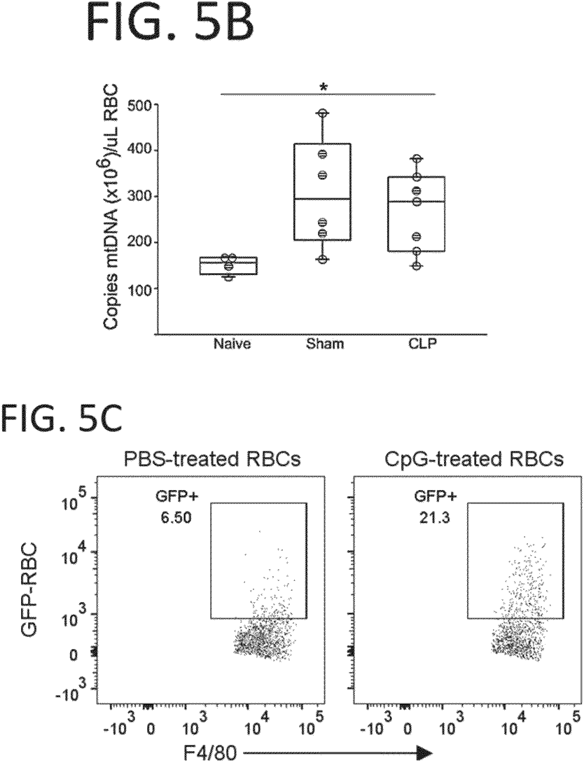

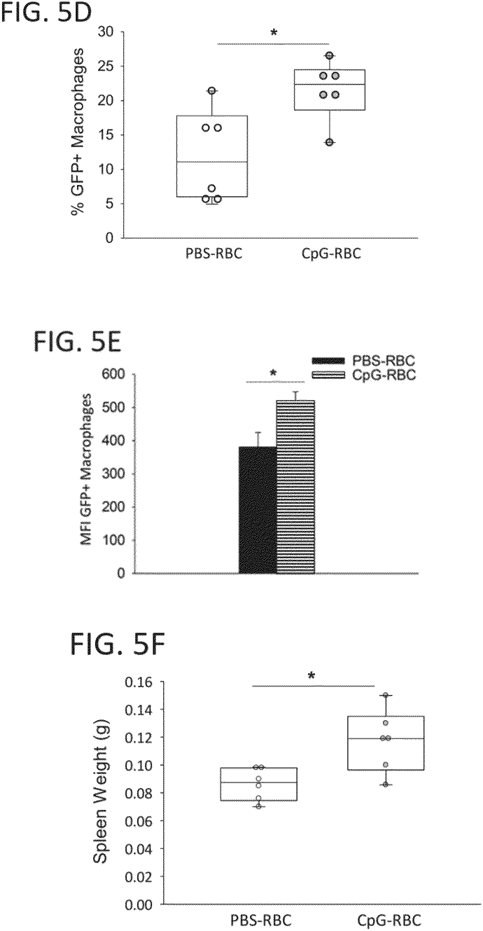

Clinical epidemiologic studies have demonstrated elevated cell free CpG containing mtDNA in the circulation during critical illness and elevated mtDNA in the plasma predicts mortality in sepsis and trauma (6, 10, 25). We measured mitochondrial DNA (coxI) in the plasma and on red blood cells in several murine models of infection (Legionella pneumophila, influenza PR8, T gondii) and in a cecal-ligation puncture (CLP) model of peritoneal sepsis. As seen in FIG. 5a, mtDNA was elevated on RBCs when compared with plasma in all models assessed. Furthermore, RBC-bound mtDNA was elevated in a model of peritoneal sepsis (FIG. 5b). Consistent with our previous findings of mtDNA acquisition by RBCs during sterile inflammation, these data indicate that CpG is sequestered on RBCs during pneumonia, parasitic infection and polymicrobial infection (14).

### CpG-Treated RBCs Undergo Accelerated Erythrophagocytosis in Naive Mice

Because CD47 is a marker of self, and loss of CD47 leads to accelerated erythrophagocytosis by red pulp F4/80 positive splenic macrophages (RPM) (26), we asked whether CpG binding by RBCs would result in accelerated clearance of RBCs. GFP-expressing RBCs were treated with PBS or CpG DNA for 2 hours prior to being washed and infused into mice. Analysis of spleens one hour following transfusion revealed that F4/80 high macrophages ingested GFP RBCs. Mice that received CpG-treated RBCs demonstrated significantly higher percentage of erythrophagocytic macrophages than mice that received PBS-treated RBCs (FIGS. 5c-e, see FIG. 11 for gating strategy). Spleen weights of animals receiving CpG-treated RBCs were also elevated at 20 hours post-infusion compared with animals that received PBS-treated RBCs, consistent with increased erythrophagocytosis and splenic congestion (FIG. 5f). Collectively, these findings demonstrate that exposure of RBCs to high concentrations of CpG DNA leads to accelerated erythrophagocytosis in vivo.

### CpG-Carrying RBCs Increase Systemic Inflammation

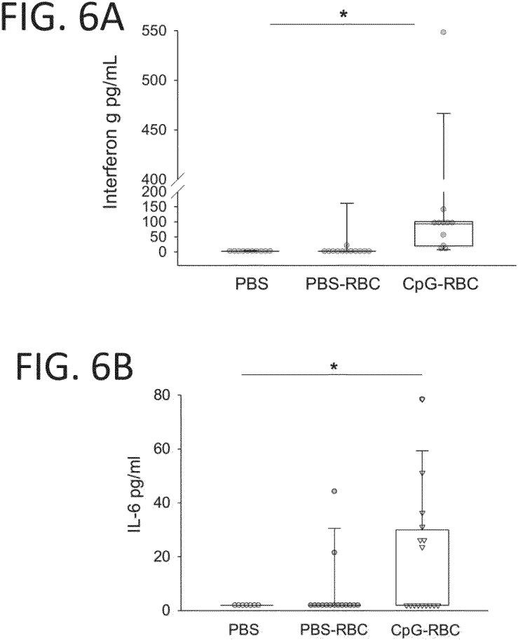

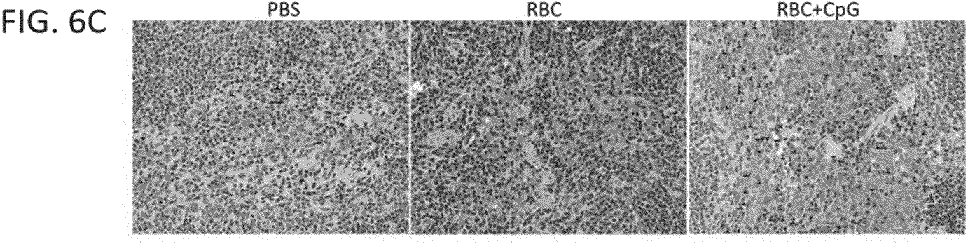

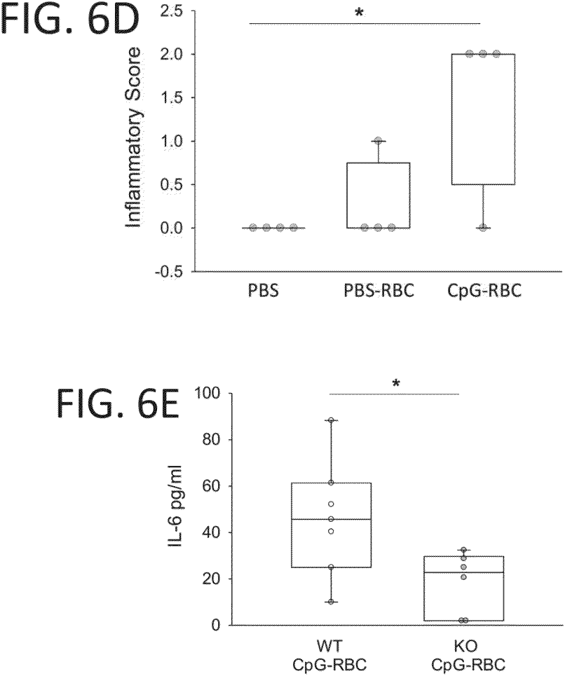

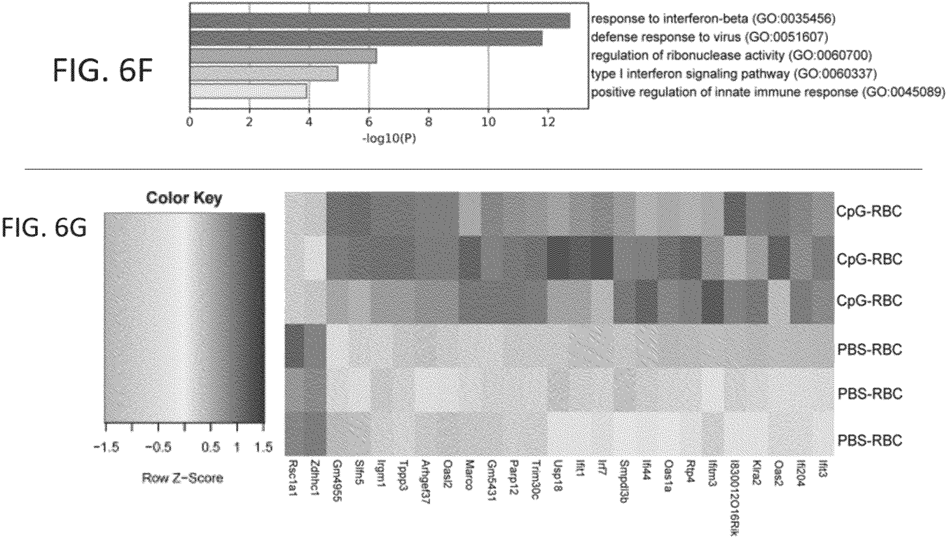

Prior studies have shown that CD47 negative erythrocytes can activate dendritic cells and serve as an adjuvant for T cell responses (27), we next asked whether the administration of RBCs treated with high doses of CpG DNA (150 uM/ 1 billion RBCs) would lead to systemic immune activation in WT mice. This was indeed the case since we found increased plasma interferon gamma and IL-6 6 hours post infusion of CpG-treated, but not PBS-treated, RBCs (FIGS. 6a&b). Splenic histology 6 hours following CpG-RBC (but not PBS treated RBCs) infusion also revealed increased neutrophil infiltration and enhanced red pulp congestion (FIGS. 6c&d). We next assessed the contribution of RBC-TLR9 to the systemic inflammatory response by treating WT and TLR9 KO RBCs with CpG for two hours prior to administration to naïve mice. Administration of CpG-treated WT RBCs resulted in elevated plasma cytokines when compared with CpG-treated KO RBCs. Collectively, these findings suggest that CpG presentation by RBCs is a substantial contributor to systemic immune responses (FIG. 6e). In order to further characterize the immune response following CpG-RBCs, we performed RNA Seq on spleens from mice treated with RBCs or CpG-treated RBCs. FIG. 12 lists the top differentially expressed genes. Consistent with our findings of elevated systemic interferon gamma following CpG-RBCs, CpG-RBCs elicited a transcriptomic response characterized by increased expression of interferon signaling pathway genes when compared with PBS-treated RBCs (FIGS. 6f&g).

### RBC TLR9 Surface Expression and mtDNA Content Is Elevated in Anemic Septic Patients

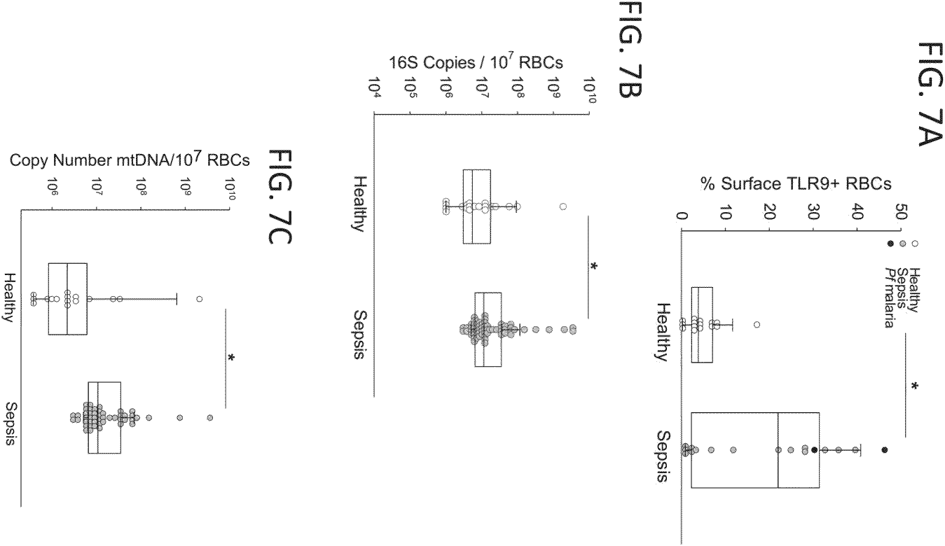

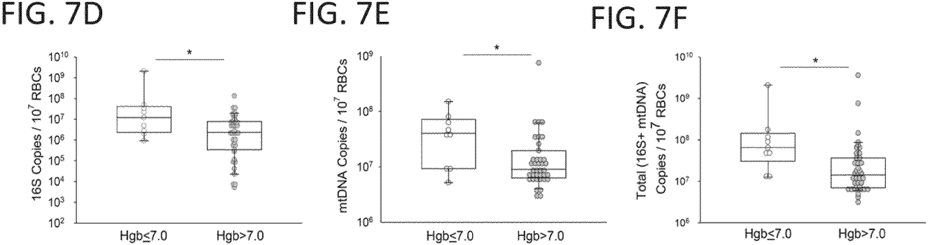

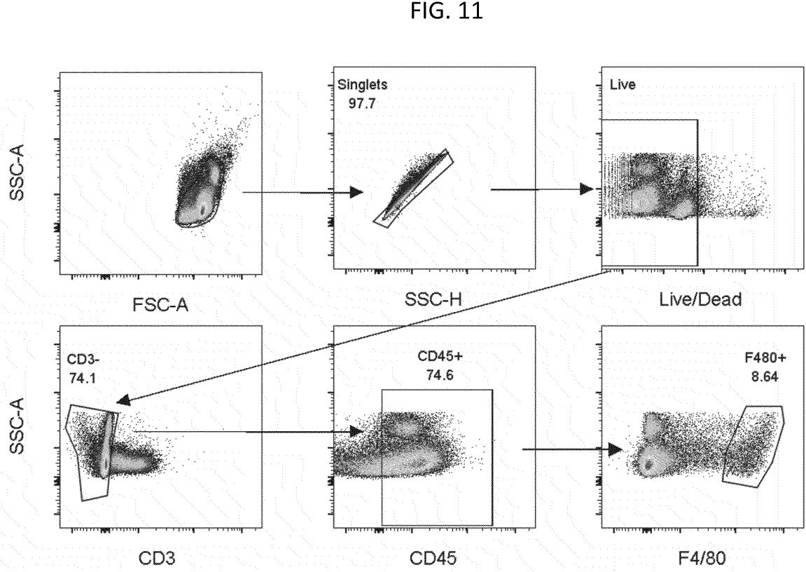

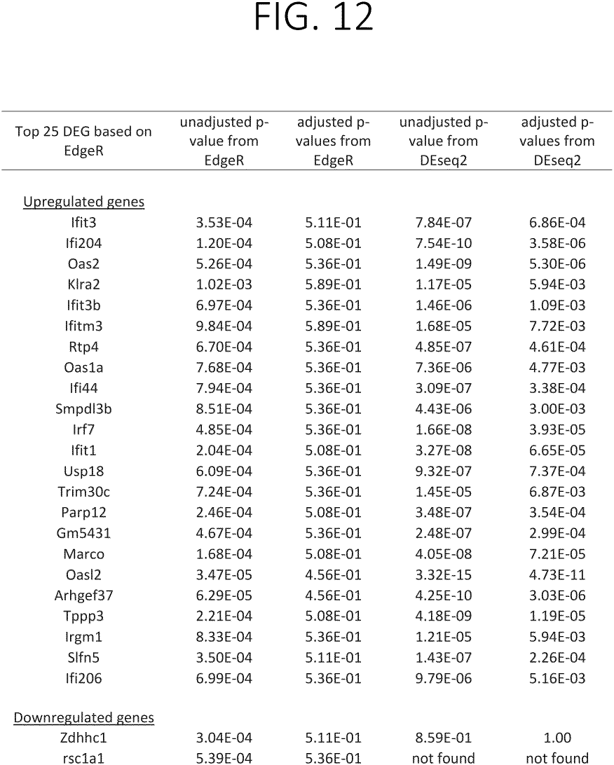

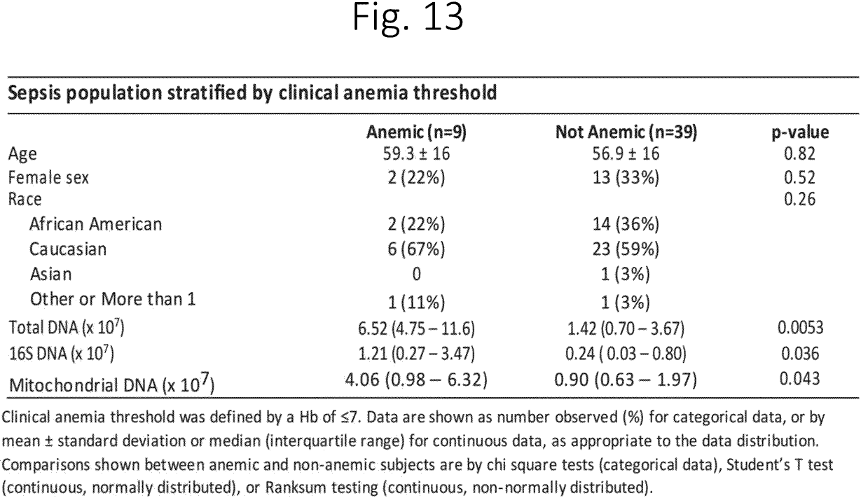

Cell-free CpG-containing mitochondrial DNA is elevated in sepsis patients and was recently found to be associated with mortality in this population (10). We thus asked whether surface expression of TLR9 would differ between healthy individuals and sepsis patients. RBCs were prospectively collected from patients enrolled in a cohort designed to study sepsis (Molecular Epidemiology of SepsiS in the ICU, MESSI cohort) at the University of Pennsylvania. Flow cytometry for TLR9 was performed on intact non-permeabilized RBCs. We found that surface TLR9 was significantly increased on RBCs from patients with sepsis when compared with RBCs from healthy donors (FIG. 7a). Elevated cell-free CpG-containing DNA is common in sepsis, and plasma mitochondrial DNA levels are also associated with increased mortality in the critically ill (10, 28). Because we have previously demonstrated that mtDNA is detectable on human RBCs, we performed quantitative PCR (qPCR) to determine the mtDNA content of RBCs obtained from healthy volunteers as well as critically ill patients with sepsis (FIG. 13) (10, 14). We found that mtDNA was increased on RBCs from patients with sepsis when compared with RBCs from healthy controls (FIG. 7b). We also measured 16 s content on RBCs by qPCR. Consistent with our in vitro findings of bacterial DNA acquisition by RBCs (FIG. 1), 16 s is elevated on RBCs during human sepsis.

Anemia is common in critically ill patients with sepsis and alterations in RBC morphology function and survival have been observed in human sepsis (29-31). Given our findings of accelerated clearance of DNA-treated RBCs in mice, we asked whether mtDNA content on RBCs would differ between anemic septic patients and non-anemic septic patients. A hemoglobin threshold of 7 g/dL was used to define anemia based on current standard of care and transfusion guidelines for critically ill patients with sepsis (32, 33). As seen in FIGS. 7d-f, anemic septic patients had higher RBC associated CpG content than non-anemic septic patients, paralleling the in vivo findings of accelerated erythrophagocytosis of DNA carrying RBCs in experimentally transfused mice.

### Discussion

In this study, we identified a new role of RBCs in the immune response to infection and the pathogenesis of anemia. We show that RBCs express surface TLR9 that can bind CpG-containing cell-free DNA that circulates in the plasma. Under basal conditions with low levels of cell-free DNA, RBCs bind CpG DNA and act as a “sink” without undergoing overt morphological changes (14). However, when there are high levels of plasma CpG DNA, such as during sepsis, sterile inflammation, or malarial infection, TLR9-dependent CpG DNA binding leads to fundamental alterations of RBC morphology. DNA binding was also associated with a functional loss of the marker of self, CD47, on a subset of RBCs since high extracellular DNA levels induced a conformational change in this protein, resulting in loss of its antiphagocytic epitope.

Our data show that DNA binding to RBC results in accelerated erythrophagocytosis. Surprisingly, CpG-binding to RBCs was sufficient to cause accelerated clearance in naïve mice. This may have implications for malaria pathogenesis since excess parasite DNA in the plasma of infected individuals may result in the removal and destruction of both infected and uninfected RBCs. Indeed, a recent study of Plasmodium berghei infected mice showed that a disruption of CD47-Sirpα binding led to accelerated erythrophagocytosis, thus implicating this interaction in the host response to malaria (34, 35). Similarly, Plasmodium DNA and TLR9 have been shown to promote autoimmune anemia through a T-Bet+ B cell mediated anti-erythrocyte antibody production in Plasmodium yoelli infected mice (36). Malarial anemia and non-malarial infectious anemia may also arise through the development of inflammatory hemophagocytes or the generation of other erythrophagocytic macrophage subsets. In mice, CpG-TLR9 interactions have been shown to promote inflammatory anemia during P. yoelli blood stage infection, and nucleic acid sensing TLRs promote anemia in a hemophagocytic lymphohistiocytosis model (12, 13). Our findings also provide a mechanism by which CpG-containing DNA promotes anemia during sepsis by resulting in the accelerated clearance of RBCs that bind this DNA. Targeting RBC-TLR9 with blocking antibodies or antagonistic small molecule inhibitors may be a viable option to combat inflammatory anemia. This could potentially eliminate the enhanced CpG-TLR9 mediated RBC phagocytosis that occurs following the loss of CD47 without interfering with CpG-TLR9 signaling in classical immune cells that are essential for host defense.

In addition to ingestion by macrophages, CpG carrying RBCs may also be taken up by dendritic cells, which may alter antigen presentation and acquired immune responses. Indeed, previous studies demonstrated that even a small fraction (0.5%) of CD47 negative RBCs was able to activate splenic dendritic cells and CD4+ T cells (27, 37, 38). Our findings of increased interferon production and decreased systemic cytokines in mice receiving TLR9 KO RBCs would suggest that RBCs can present CpG to immune cells. Indeed, we detected CpG on RBCs during parasitic, bacterial, viral infection and sterile inflammation. Furthermore, CpG-carrying RBCs induced an innate immune response in naïve mice as administration of CpG-carrying RBCs led to a transcriptomic response in the spleen characterized by host response to virus, innate immune response and interferon signaling pathways. Thus, RBCs may contribute to the immune dysregulation that has been observed during inflammatory states, including sepsis by presenting CpG to immune cells. Although beyond the scope of this study, analysis of the erythrophagocytic cells on a single cell level will be critical in elucidating the exact mechanisms of innate immune activation by CpG-carrying RBCs.

TLR9 is expressed on nucleated erythrocytes in other vertebrates, including fish (39). Birds express the avian homolog of TLR9, TLR21, on their erythrocytes (39). Here, we demonstrate the presence of TLR9 and DNA binding by human, chimpanzee and murine RBCs and show a role for RBCs in sensing CpG DNA, a potent activator of the innate immune system. Humans produce over 2 million RBCs each second and are at risk for exposure to large amounts of DNA during mitophagy and nuclear expulsion. Thus, it is tempting to speculate that TLR9 is retained on erythrocytes to protect RBCs during erythroid maturation by scavenging mitochondrial DNA that escapes mitophagy. Indeed, recent studies have shown that loss of mitophagy leads to RBC destruction and anemia, and other studies have demonstrated that mtDNA that escapes mitophagy leads to cell-autonomous TLR9 mediated inflammation (40, 41). Alternatively, given the inflammatory response observed following administration of CpG treated RBCs to naïve mice is plausible to speculate that retention of TLR9 on RBCs promoted host survival by allowing for propagation of local signals remotely and early innate detection of cell free DNA released during infection or following trauma. While further studies will be required to elucidate the role of TLR9 in erythroid development, our findings of RBC mediated nucleic acid sensing confirm a role for TLR9 on mature erythrocytes in regulating the immune response during acute inflammation.

Our data demonstrate that red cells serve as DNA sensors through surface expression of TLR9, which appears to be beneficial during quiescent states, where it promotes scavenging of trace levels of CpG to prevent non-specific inflammation (14). However, during states of excess circulating CpG, such as sepsis, parasitic infection or trauma, binding of CpG by TLR9 leads to accelerated clearance of RBCs and inflammation. We speculate that this immune activation may be beneficial in the clearance of bacterial and parasitic infection. On the other hand, this innate immune mechanism contributes to the inflammatory response and development of anemia during pathologic states where cell free DNA is elevated. Thus, DNA recognition by TLR9 on RBCs provides bona fide evidence for red cells as immune sentinels and links the immune response with anemia.

### Example 3: Human RBC Bind to Bacterial DNA

It was first demonstrated that 16S Ribosomal RNA gene DNA (a conserved gene present in bacteria 16S) was detectable on RBCs and elevated in sepsis patients when compared with healthy subjects (FIG. 7B, FIG. 21). However, 16S did not distinguish clinical outcomes (FIG. 22), and sequencing of 16S from RBCs did not reveal any differences in pathogen detection between bacteremic and non-bacteremic patients with sepsis. We therefore concluded that NGS methods for pathogen detection are costly and will be ineffective if RBCs are used as a substrate.

1×10⁷ human RBC from sepsis patients or healthy donors were incubated with 10 ng or 1 ng bacterial DNA in 200uL at 37C with gentle shaking at 90 rpm. Cells were overlaid on 500uL 30% sucrose cushion and spun at 13000 g at 4C for 3 min. Cell pellets were collected, and DNA was extracted using Qiagenblood DNA kit. qPCRwas performed using bacterial specific primer/probe set.

A standard curve was generated using corresponding bacterial genomic DNA to quantify the DNA content in our studies. Plots for K. pneumoniae, P. aeruginosa, and S. pneumoniae are shown in FIGS. 14-16 respectively.

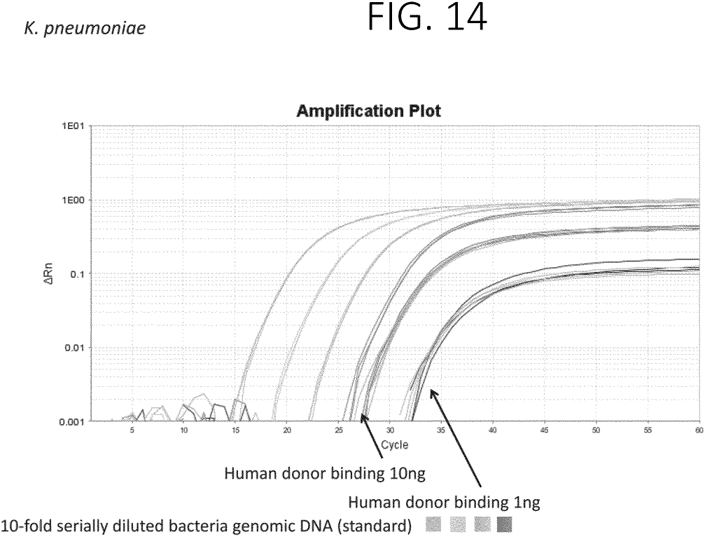

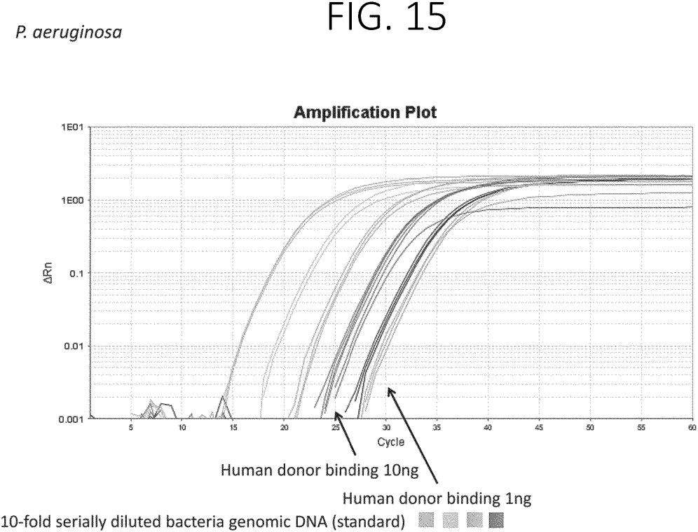

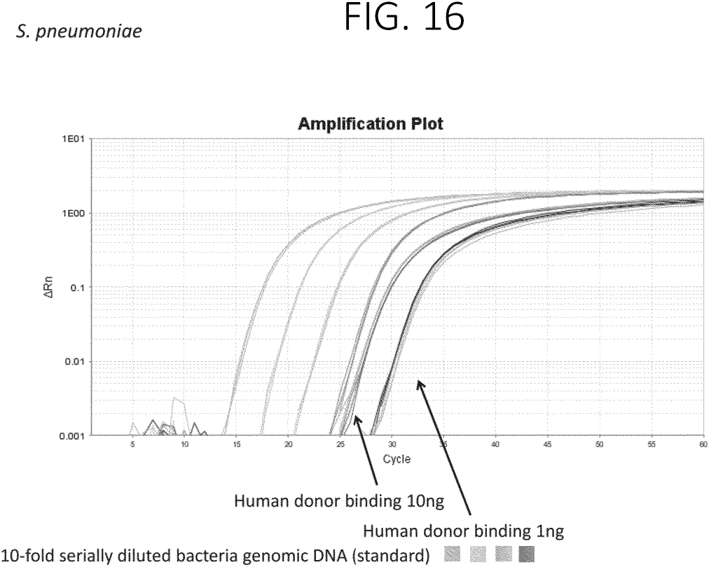

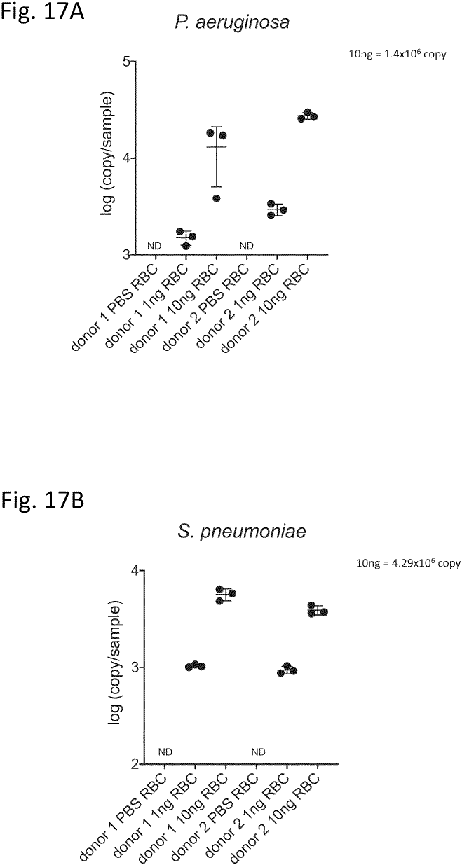

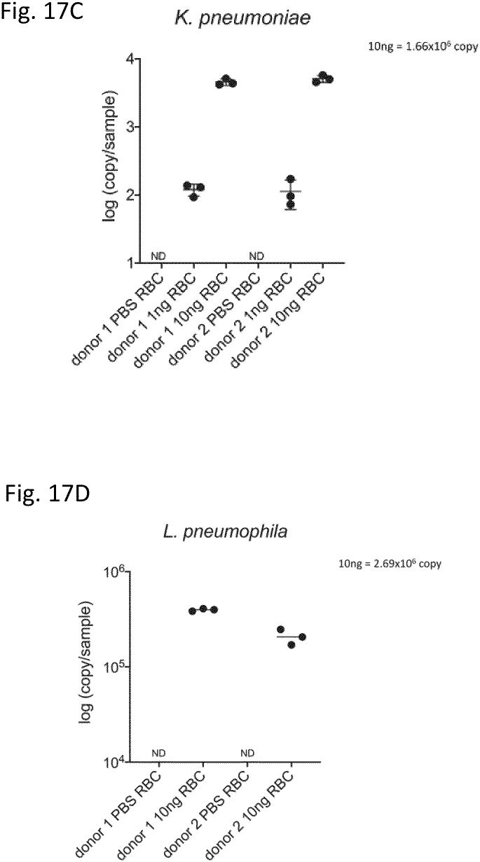

DNA from 1×10⁷ human RBC from sepsis patients or healthy donors were extracted using Qiagen blood DNA kit. qPCRwas performed using bacterial specific primer/probe set. A standard curve was generated using corresponding bacterial genomic DNA to quantify the DNA content in our studies. P. aeruginosa, S. pneumoniae, K. pneumoniae, and S. aureus levels in healthy controls and patients were compared. FIGS. 17-18 show that bacteria are detected on patient RBCs.

### Example 4: Measurement of RBC-Complement in a Curated Cohort of COVID Patients

Several pathophysiological features of COVID-ARDS are striking, unique and lead us to propose that SARS-CoV-2 causes a vascular endotype of ARDS. Remarkably, the predisposing causes for respiratory decompensation include cardiovascular disease, HBP and diabetes rather than more traditional risk factors such as asthma. Clinical observation and autopsy data from COVID-19 patients with respiratory failure demonstrate extensive injury to the lung microvasculature, with enlarged capillaries, immune complex and complement deposition and endothelial cell (EC) death. Injury to the lung microvasculature may impede blood flow due to vascular occlusion or loss of vasoregulation, leading to ventilation/perfusion mismatch, hypoxia and clinical deterioration. These observations suggest that dysfunction of the pulmonary vasculature is a central driver of COVID-induced ARDS.

Moreover, the incidence of thromboembolic complications (TECs) in COVID-19 patients is truly extraordinary and considerably exceeds the incidence in ICU patients in general and those with other causes of ARDS. Thrombi occur in the deep venous system but also within the arterial circulation leading to stroke, myocardial infarction, occlusion of dialysis catheters, ECMO circuits, etc. (our clinical unpublished observations). The predominant site of TECs by far is the lung. Autopsy studies demonstrate in situ thrombi in association with intense vascular inflammation. Therefore, targeted therapies that supplement anticoagulation are desperately needed to mitigate thromboembolic complications and development of ARDS.

One potential reason for limited benefits seen with anticoagulation comes from emerging literature that suggests SARS-CoV-2 can infect pulmonary endothelial cells (ECs), activate complement through direct interactions of N protein with lectin pathway components and induce EC death^{42,} ⁴⁸. Evidence with Cov1 indicates a role for autoreactive antibodies and viral-containing immune complexes in endothelial injury as well. The late respiratory decompensation seen in Covid-19 patients is consistent with antibody and complement mediated endothelial injury and proposals for widespread use of complement inhibitors has been advanced. However, complement may be important in host defense against the virus and patient selection for such intervention is hampered by the lack of a relevant biomarker. Plasma C3, C4 and CH50 are often normal with injury that occurs at the tissue level. Moreover, the potential for convalescent plasma to contain complement-activating EC antibodies has not been considered. In addition to a lack of rationale targeted therapeutics and off-target effects, one of the main barriers to trial design is patient heterogeneity, i.e., the inability to predict which and when patients might benefit from intervention. COVID-ARDS provides a unique endotype in which the timing of respiratory failure likely resulting from immune endothelial injury has been defined.

COVID 19, the disease caused by the SARS-CoV-2 virus, can progress to multiorgan failure characterized by respiratory insufficiency, arrhythmias, thromboembolic complications and shock⁴⁴⁻⁴⁸. The mortality of patients hospitalized with COVID 19 is unacceptably high and new strategies are urgently needed to rapidly identify and treat patients at risk for organ failure. Clinical epidemiologic studies demonstrate that vulnerability is greatest after viral clearance from the upper airway,⁴⁹⁻⁵¹ which suggests that dysregulation of the host immune response is a critical mediator of clinical deterioration and death. Autopsy and pre-clinical evidence implicate aberrant complement activation in endothelial injury and organ failure⁵²⁻⁵³. Several COVID 19 patients have received inhibitors of complement, ⁵³⁻⁵⁵ but biomarkers to identify the optimal timing and candidates for therapy are lacking. Here we report the presence of complement containing immune complexes on circulating erythrocytes from critically ill COVID 19 patients using flow cytometry. These findings indicate that novel erythrocyte-based diagnostics provide a method to identify patients with dysregulated complement activation. RBC-based diagnostics utilize only a small amount of blood and are amenable to rapid point of care testing.

An ancient arm of innate immunity, the complement system is a front-line defender against invading pathogens. This multitiered and highly coordinated system is vital for the innate immune response to pathogens, removal of dead cells and maintenance of homeostasis. Initially described as a complement to antibody mediated immunity, it was later discovered that the complement system can be deployed by lectins and membrane associated mediators bound to pathogenic surfaces. Thus, the three arms of the complement system can be engaged by distinct initiators including antigen-antibody complexes (classical pathway), lectins (lectin pathway) and C3 hydrolysis (alternate pathway). Although the complement system promotes clearance of pathogens through opsonization, inflammation and cytolysis, dysregulated complement activation can lead to cellular injury, microvascular thrombosis and organ failure.

Complement-containing immune complexes bind to cells through a number of specific receptors, including complement receptor 1 (CR1), which recognizes complement activation products C3b, etc. Most CR1 in the human body is expressed on the surface of the nearly thirty trillion erythrocytes (red blood cells, RBCs) in the circulation. Plasma-based assays of individual complement components or total complement activity may fail to reflect complement activation happening on cell surfaces that is responsible for tissue injury (aHUS). We demonstrate that circulating RBCs provide a sensitive measure to detect complement activation that may be occurring at the tissue level in critically ill patients with COVID 19.

We have performed flow cytometry on RBCs from ~65 sepsis patients in the Molecular Epidemiology of SepSIs cohort (MESSI) and have collected over 20 RBC samples from patients with COVID-19 and we continue to enroll patients daily.

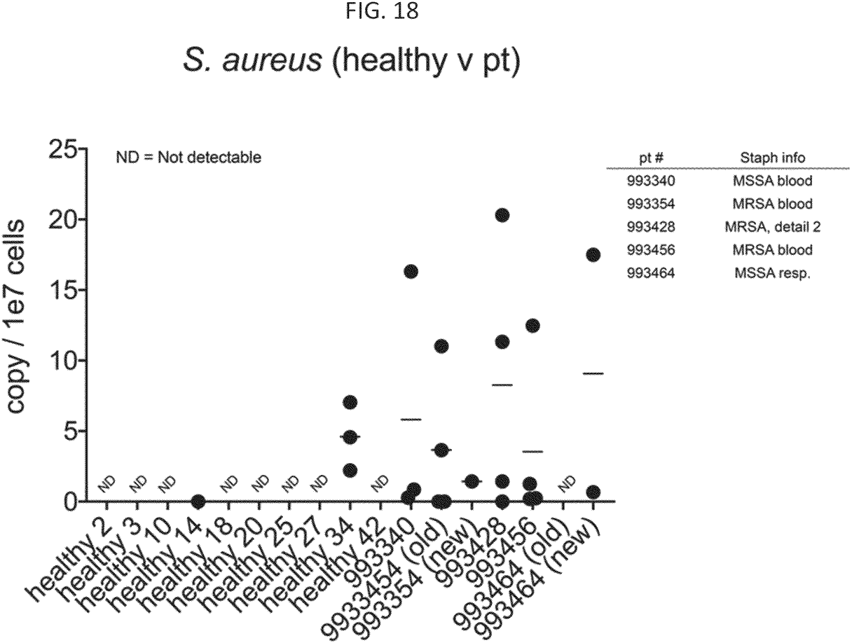

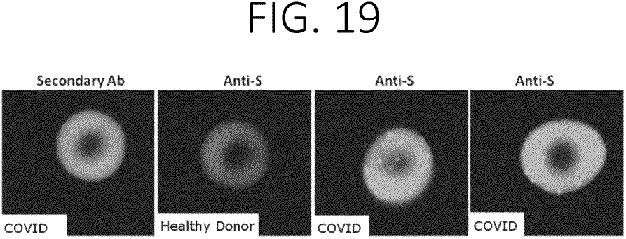

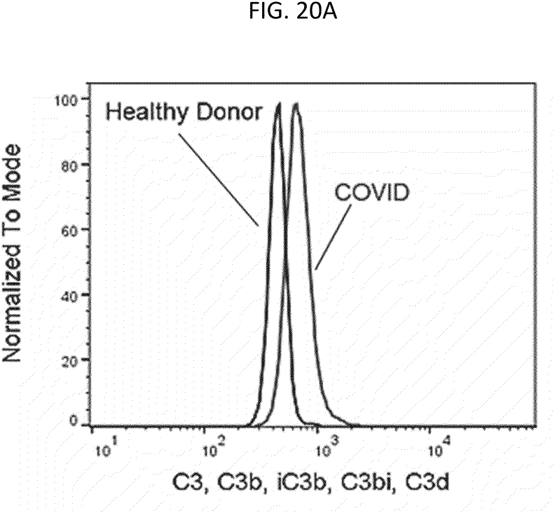

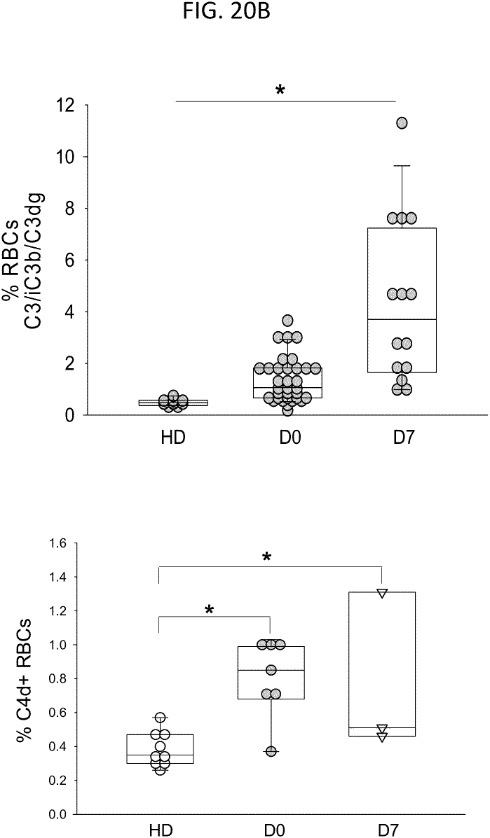

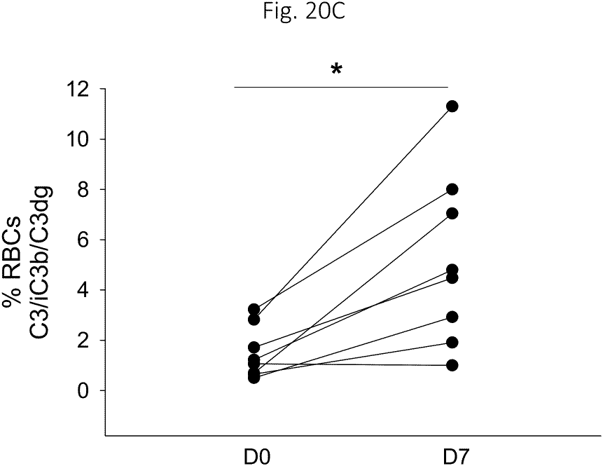

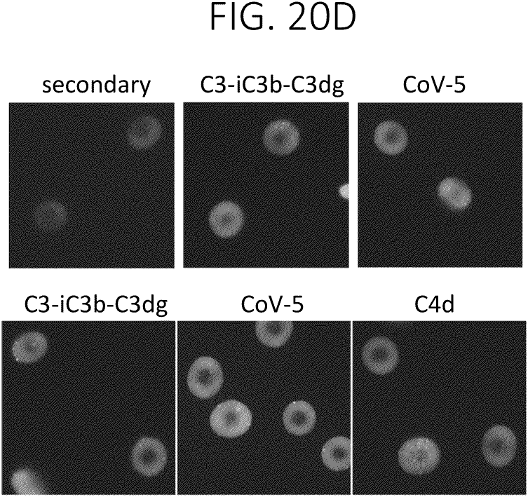

Flow cytometry was used to measure viral spike (FIG. 19) and N protein, IgM, IgG and complement fragments on RBCs from residual blood collected for clinical purposes (FIG. 20A). We performed flow cytometry on RBCs taken on admission and on day 2 and day 7 after ICU admission to determine the relationship between intensity and duration of viral antigen carriage, the onset of immune complex/complement activation and clinical status determined retroactively. We have utilized residual RBCs for immunoassays in the past, which has not affected our ability to detect RBC bound chemokine, mtDNA or HMGB 1.

RBCs from healthy donors (HD) or critically ill COVID-19 patients were collected and isolated from whole blood (EDTA or heparin tubes). Fluorescently conjugated mouse monoclonal Ab to C3 (C3, iC3b, C3dg) were utilized to detect complement fragments on RBCs (FIG. 20B). Fluorescently conjugated mouse monoclonal Ab to viral spike protein were utilized to detect viral fragments on RBCs. Further, viral spike protein was detected by flow cytometry on RBCs from critically ill patients with COVID-19 but not on healthy donor RBCs using as little as 2 microliters of blood. RBCs from COVID patients (d0 and d7 of ICU admission) bind complement fragments. d7 is significantly higher than d0, p=0.007) (FIG. 20C).

Immunofluoresence was performed. iC3b, C3dg and C4d were detectable on the RBCs from COVID patients (FIG. 20D). Spike (low levels) detected. NP was not detected. PCR for SARS-CoV-2 RNA performed on RBCs did not detect any RNA. Punctae were observed in the day 7 RBCs of the two critically ill COVID patients (FIG. 20D).

The % C3 fragments detected by flow were 0,0,11,7 for HCW, 994555,994563, 994565.

Quantification of punctae, improved imaging, IP of immune complex and correlation with FACS are performed.

To detect complement activation in patients with COVID 19, we measured erythrocyte-bound C3, iC3b, C3dg using flow cytometry. RBCs were obtained from healthy donors (HD) or critically ill patients with COVID 19. Blood samples from patients were obtained from EDTA tubes on day 0 and day 7 of admission to the ICU. The percentage of RBCs expressing C3/iC3b/C3dg was markedly elevated in critically ill COVID 19 patients admitted to the ICU when compared with HD and rose even further by day 7 (FIG. 20B). Immunofluorescence staining revealed that COVID 19 erythrocytes not only expressed C3 fragments, but also expressed viral spike protein, IgM and C4d, suggesting activation of the classic pathway of complement and deposition of immune complexes on the RBCs (FIG. 20B). In contrast, neither viral NP protein nor viral RNA were detected on erythrocytes from critically ill COVID 19 patients. Together, these data indicate that complement-fixing immune complexes are present in the circulation in patients with COVID 19 associated organ injury.

We next examined the association of RBC-bound complement, CRP, D Dimers and clinical outcomes. The presence of IgM as well as fragments of C4 and C3 on patient RBCs suggests the presence of immune complexes leading to activation of the classic pathway of complement. However, direct binding of the N protein of (SARS-CoV1 or SARS-CoV2) to MASP-2 (Mannan-binding lectin serine protease 2) may also lead to activation of the lectin pathway.⁵³ Polymorphisms in the MBL protein, which initiates the lectin pathway, and mutations or dysfunction of complement regulatory proteins have been implicated in diverse disorders characterized by intravascular thrombosis. Furthermore, risk factors for COVID-associated lung injury such as diabetes have been associated with high circulating levels of MBL and dysfunctional complement regulatory proteins on the endothelium and erythrocyte.⁵⁶⁻⁶⁰

The implications of our findings are several-fold. First, flow cytometry of RBCs is a clinically relevant and readily accessible test that may provide a sensitive measure of complement activation that reflects events occurring at the tissue level. Second, an increase in RBC-complement over time might help to identify patients who are at risk for complement-mediated tissue injury and thereby identify potential candidates for clinical trials of anti-complement therapy. Third, the effects of complement on RBC physiology may contribute directly or serve as a marker of the propensity for complement-mediated microvascular thrombosis that contributes to hypoxemia and multiorgan failure.

### Example 5: Multiplexed Assay

We will identify 50-100 patients from the MESSI cohort with radiograph confirmed pneumonia. We have developed a PCR based method of testing RBCs for bacterial pathogens. This method requires 10^7 RBCs (<5 uL of whole blood). We test the ability of the RBC based PCR to detect pathogen in patients with pneumonia. RBC based diagnosis is compared with respiratory culture.

We utilize a multiplex assay using 16S PCR with pathogen-specific probes for the 6 most common morbidity-associated pneumonia pathogens (Staph aureus, Strep pneumoniae, Klebsiella pneumonia, Pseudomonas aeruginosa, E coli, Legionella pneumophilia). We compare multiplex test performance against respiratory culture.

Bacterial DNA binding to human RBCs: S. aureus bacteria (25923D-5) and P. aeruginosa genomic DNA (47085DQ) were obtained from ATCC. Bacterial culture were gifts from Dr. Sunny Shin (L. pneumophila, University of Pennsylvania), Dr. Hao Shen (S. pneumoniae, University of Pennsylvania), and Dr. G Scott Worthen (K. pneumonia, University of Pennsylvania). Bacterial genomic DNA was isolated and purified from bacteria using DNeasy DNA blood & tissue kit (Qiagen). 1×10⁷ RBCs from healthy donors were incubated with 0 ng (PBS), 0.1 ng, 1 ng, or 10 ng purified DNA in 2 hr at 37° C. in gentle shaking in DNA LoBind tubes (Eppendorf) and washed with PBS twice. RBC-associated DNA was extracted from samples using DNeasy kit (Qiagen), and genomic DNA were quantified with qPCR using primer or probes in the table below.

| | | | |
|---|---|---|---|
| Target | Forward Primer (5′->3′) | Reverse Primer (5′->3′) | Probe (5′->3′) |
| S. aureus (16S) | TCGGMTCGTAAA ACTCTGTT (SEQ ID NO: 29) | CTGCTGGCACG AAGTTAGC (SEQ ID NO: 30) | /56-FAM/AAGAACATA/ZEN/TGT GTAAGTAACTGTGCACA/3I ABkFQ/(SEQ ID NO: 31) |
| K. pneumo niae (16S) | GCCTTCGGGTTGT AAAGY (SEQ ID NO: 32) | CTGCTGGCACG AAGTTAGC (SEQ ID NO: 33) | /56-FAM/TTAATAACC/ZEN/TYR KCGATTGACGTTACCC/3IA BkFQ/ (SEQ ID NO: 34) |
| L. pneumo phila (16S) | TACCTACCCTTGA CATACAGTG (SEQ ID NO: 35) | CTTCCTCCGGTT TGTCAC (SEQ ID NO: 36) | /56-FAM/GAGTCCCCA/ZEN/CCA TCACATG/3IABkFQ/ (SEQ ID NO: 37) |
| P. aerugin osa (16S) | CTGGAAGCAGGA TGGCTATT (SEQ ID NO: 38) | CAGTAGCGGGA AGAGAATGTAG (SEQ ID NO: 39) | /56-FAM/AACTGCTCT/ZEN/TCC ACCGACAACGAC/31ABkFQ/ (SEQ ID NO: 40) |
| S. pneumo niae (CpsA) | GCTGTTTTAGCAG ATAGTGAGATCG A (SEQ ID NO: 41) | TCCCAGTCGGT GCTGTCA (SEQ ID NO: 42) | /56FAM/AATGTTACGCAACT GACGAG/3IABkFQ/ (SEQ ID NO: 43) |
| 16S (univer sal) | BSF8:AGAGTTGAT CCTGGCTCAG (SEQ ID NO: 44) | BSR357:CTGCTG CCTYCCGTA (SEQ ID NO: 45) | /56-FAM/TAA+CA+CATG+CA+A GT+CGA/3BHQ_1/ (SEQ ID NO: 46) |

FIG. 25 demonstrates the results of a multiplex PCR identifying Staph aureus, Strep pneumoniae, Klebsiella pneumonia, Pseudomonas aeruginosa, and Legionella pneumophilia on the RBCs of 4 healthy donors.

### Example 6: Presence of Viral Receptor on RBCs

TLR7 staining: Whole blood of healthy human donors were collected in EDTA tubes and centrifuged at 3000 g for 10 min at deceleration 6. After removal of plasma and buffer coat, RBCs were isolated using leukoreduction filters (Acrodisc). 1×10⁶ RBCs were resupended in PBS, fixed in 0.05% glutaraldehyde for 10 min, and permeabilized in 0.1% tritonX-100. Cells were washed and resuspended in FACS buffer (PBS + 2% FBS).

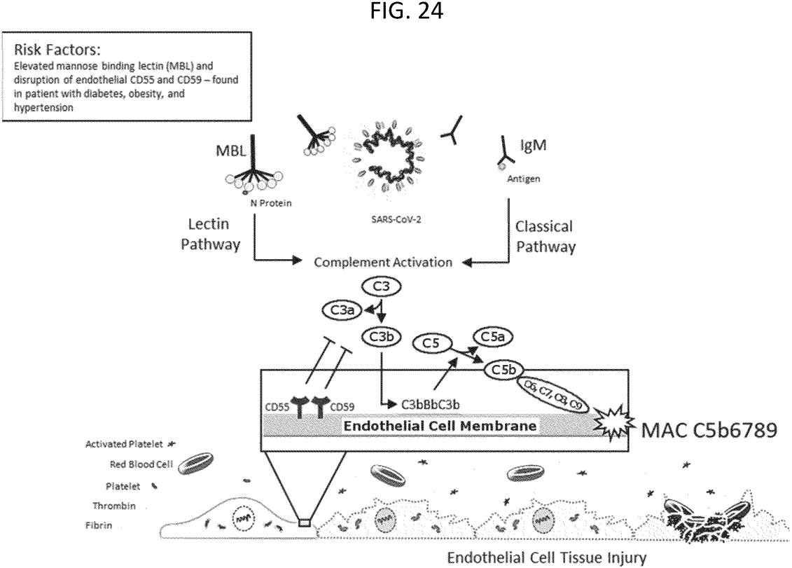

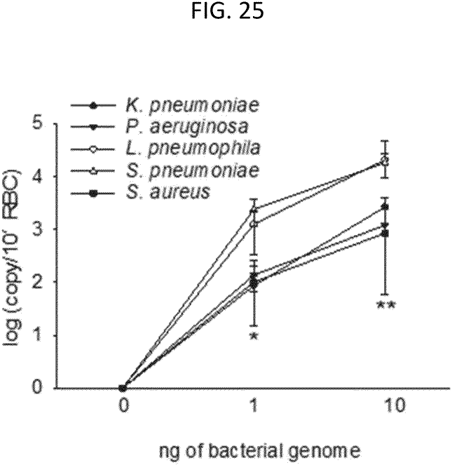

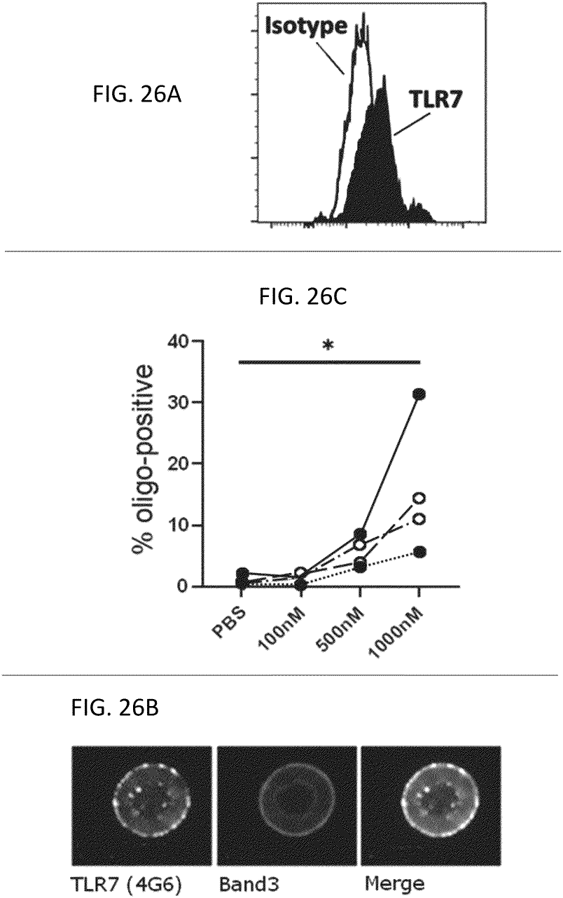

To determine TLR7 expression with FACS, 250,000 RBCs were blocked in FACS buffer with 5% goat serum for 1 hr, stained in 5ug mouse anti-TLR7 (Novus biological, clone 4G6) or isotype antibody in 100uL for 1 hr at room temperature (RT), washed thrice, and probed with PE goat-anti-mouse secondary antibody for 30 min. After washes, samples were analyzed with BD Fortessa flow cytometer. FIG. 26A demonstrates the presence of TLR7 on RBCs.

To visualize TLR7 expression in RBCs, 500,000 RBCs were blocked in goat in staining buffer (PBS + 1% BSA) with 5% goat serum for 1 hr, stained in anti-TLR7 (4G6) at 5ug/mL and rabbit anti-band3 (Abcam, ab 108414) at lug/mL overnight at 4C in staining buffer. Cells were washed in PBS +0.05% tween20 and stained with secondary antibodies in staining buffer for 1 hr at RT. After 3 washes, cells were resuspended in 10uL of PBS, mounted on glass slides with 2uL fluoromount G, and sealed with coverslip and nail polish. Images were taken with Nikon A-2 microscope. FIG. 26B reveals surface staining on RBCs, demonstrating presence of TRL7.

Binding of RNA40 on RBC: RBCs isolated as above were resuspended in PBS after leukoreduction (before fixation/permeabilization). 250,000 RBCs were incubated with the TLR7-stimulating oligoribonucleotide, RNA40 (5′-GsCsCsCsGsUsCsUsGsUsUsGsUsGsUsGsAsCsUsC-/ATTO647/-3′ (SEQ ID NO: 47), where “s” depicts phosphothioate linkage), at indicated concentrations at 200uL per reaction volume. Oligo binding was carried in DNA lo-bind tubes. The reactions were incubated at 37C on a nutator for 2 hr. Cells were washed in PBS thrice prior to analysis by flow cytometry.

FIG. 26C demonstrates viral nucleic acid binding to RBCs by flow cytometry. The RNA40 sequence from HIV1 bound to RBCs. Each line represents an individual donor.

### REFERENCES

1. K. Neote, W. Darbonne, J. Ogez, R. Horuk, T. J. Schall, Identification of a promiscuous inflammatory peptide receptor on the surface of red blood cells. J Bioi Chem 268, 12247-12249 (1993).

2. W. C. Darbonne et al., Red blood cells are a sink for interleukin 8, a leukocyte chemotaxin. J Clin Invest 88, 1362-1369 (1991).

3. R. A. Nelson, Jr., The immune-adherence phenomenon; an immunologically specific reaction between microorganisms and erythrocytes leading to enhanced phagocytosis. Science 118, 733-737 (1953).

4. H. Hemmi et al., A Toll-like receptor recognizes bacterial DNA. Nature 408, 740-745 (2000).

5. F. J. Barrat et al., Nucleic acids of mammalian origin can act as endogenous ligands for Toll-like receptors and may promote systemic lupus erythematosus. J Exp Med 202, 1131-1139 (2005).

6. Q. Zhang et al., Circulating mitochondrial DAMPs cause inflammatory responses to injury. Nature 464, 104-107 (2010).

7. C. Lood et al., Neutrophil extracellular traps enriched in oxidized mitochondrial DNA are interferogenic and contribute to lupus-like disease. Nat Med 22, 146-153 (2016).

8. A. Cossarizza et al., Increased plasma levels of extracellular mitochondrial DNA during HIV infection: a new role for mitochondrial damage-associated molecular patterns during inflammation. Mitochondrion 11, 750-755 (2011).

9. L. Alexopoulou, A. C. Holt, R. Medzhitov, R. A. Flavell, Recognition of double-stranded RNA and activation of NF-_{K}B by Toll-like receptor 3. Nature 413, 732 (2001).

10. K. Nakahira et al., Circulating mitochondrial DNA in patients in the ICU as a marker of mortality: derivation and validation. PLoSMed 10, e1001577; discussion e1001577 (2013).

11. E. M. Behrens et al., Repeated TLR9 stimulation results in macrophage activation syndrome-like disease in mice. J Clin Invest 121, 2264-2277 (2011).

12. H. M. Akilesh et al., Chronic TLR7 and TLR9 signaling drives anemia via differentiation of specialized hemophagocytes. Science 363, (2019).

13. A. Wang et al., Specific sequences of infectious challenge lead to secondary hemophagocytic lymphohistiocytosis-like disease in mice. Proc Natl Acad Sci USA 116, 2200-2209 (2019).

14. M. J. Hotz et al., Red Blood Cells Homeostatically Bind Mitochondrial DNA through TLR9 to Maintain Quiescence and to Prevent Lung Injury. Am J Respir Crit Care Med 197, 470-480 (2018).

15. M. Onji et al., An essential role for the N-terminal fragment of Toll-like receptor 9 in DNA sensing. Nat Commun 4, 1949 (2013).

16. A. Eaton-Bassiri et al., Toll-like receptor 9 can be expressed at the cell surface of distinct populations of tonsils and human peripheral blood mononuclear cells. Infect Immun 72, 7202-7211 (2004).

17. J. B. Ewaschuk et al., Surface expression of Toll-like receptor 9 is upregulated on intestinal epithelial cells in response to pathogenic bacterial DNA. Infect Immun 75, 2572-2579 (2007).

18. P. Parroche et al., Malaria hemozoin is immunologically inert but radically enhances innate responses by presenting malaria DNA to Toll-like receptor 9. Proc Natl Acad Sci US A 104, 1919-1924 (2007).

19. E. Latz et al., Ligand-induced conformational changes allosterically activate Toll-like receptor 9. Nat Immunol 8, 772-779 (2007).

20. L. J. Bruce et al., A band 3-based macrocomplex of integral and peripheral proteins in the RBC membrane. Blood 101, 4180-4188 (2003).

21. M. Salomao et al., Protein 4.1R-dependent multiprotein complex: New insights into the structural organization of the red blood cell membrane. Proceedings of the National Academy of Sciences 105, 8026-8031 (2008).

22. J. E. Brittain, K. J. Mlinar, C. S. Anderson, E. P. Orringer, L. V. Parise, Integrin-associated protein is an adhesion receptor on sickle red blood cells for immobilized thrombospondin. Blood 97, 2159-2164 (2001). 23. P. Burger, P. Hilarius-Stokman, D. de Korte, T. K. van den Berg, R. van Bruggen, CD47 functions as a molecular switch for erythrocyte phagocytosis. Blood 119, 5512-5521 (2012).

24. X. Liu et al., CD47 blockade triggers T cell-mediated destruction of immunogenic tumors. Nat Med 21, 1209-1215 (2015).

25. J. D. Simmons et al., Elevated levels of plasma mitochondrial DNA DAMPs are linked to clinical outcome in severely injured human subjects. Annals of surgery 258, 591-596; discussion 596-598 (2013).

26. P. A. Oldenborg et al., Role of CD47 as a marker of self on red blood cells. Science 288, 2051-2054 (2000).

27. T. Yi et al., Splenic Dendritic Cells Survey Red Blood Cells for Missing Self-CD47 to Trigger Adaptive Immune Responses. Immunity 43, 764-775 (2015).

28. T. Sursal et al., Plasma bacterial and mitochondrial DNA distinguish bacterial sepsis from sterile systemic inflammatory response syndrome and quantify inflammatory tissue injury in nonhuman primates. Shock 39, 55-62 (2013).

29. F. Sadaka, J. O’Brien, S. Prakash, Red cell distribution width and outcome in patients with septic shock. Journal of intensive care medicine 28, 307-313 (2013).

30. N. S. Ku et al., Red blood cell distribution width is an independent predictor of mortality in patients with gram-negative bacteremia. Shock 38, 123-127 (2012).

31. C. H. Kim et al., An increase in red blood cell distribution width from baseline predicts mortality in patients with severe sepsis or septic shock. Crit Care 17, R282 (2013).

32. L. B. Holst et al., Lower versus higher hemoglobin threshold for transfusion in septic shock. N Engl J Med 371, 1381-1391 (2014).

33. A. Rhodes et al., Surviving Sepsis Campaign: International Guidelines for Management of Sepsis and Septic Shock: 2016. Crit Care Med 45, 486-552 (2017).

34. R. Banerjee, S. Khandelwal, Y. Kozakai, B. Sahu, S. Kumar, CD47 regulates the phagocytic clearance and replication of the Plasmodium yoelii malaria parasite. Proc Natl Acad Sci U S A 112, 3062-3067 (2015).

35. K. Ayi et al., CD47-SIRPalpha Interactions Regulate Macrophage Uptake of Plasmodium falciparum-Infected Erythrocytes and Clearance of Malaria In Vivo. Infect Immun 84, 2002-2011 (2016).

36. J. Rivera-Correa et al., Plasmodium DNA-mediated TLR9 activation of T-bet(+) B cells contributes to autoimmune anaemia during malaria. Nat Commun 8, 1282 (2017).

37. J. Wu, H. Wu, J. An, C. M. Ballantyne, J. G. Cyster, Critical role of integrin C11c in splenic dendritic cell capture of missing-self CD47 cells to induce adaptive immunity. Proc Natl Acad Sci USA 115, 6786-6791 (2018).

38. T. K. van den Berg, R. van Bruggen, Loss of CD47 Makes Dendritic Cells See Red. Immunity 43, 622-624 (2015).

39. D. Morera et al., RNA-Seq reveals an integrated immune response in nucleated erythrocytes. PLoS One 6, e26998 (2011).

40. T. Oka et al., Mitochondrial DNA that escapes from autophagy causes inflammation and heart failure. Nature 485, 251-255 (2012).

41. M. Mortensen et al., Loss of autophagy in erythroid cells leads to defective removal of mitochondria and severe anemia in vivo. Proc Natl Acad Sci USA 107, 832-837 (2010).

42. D. Y. Qing et al., Red blood cells induce necroptosis of lung endothelial cells and increase susceptibility to lung inflammation. Am J Respir Crit Care Med 190, 1243-1254 (2014).

43. A. K. Kirsten Moll, Arthur Scherf and Mats Wahlgren Methods in Malaria Research. A. K. Kirsten Moll, Arthur Scherf and Mats Wahlgren Ed., (Manassas, VA, USA, 2013).

44. Jordan, R. E., Adab, P. & Cheng, K. K. Covid-19: risk factors for severe disease and death. BMJ 368, m1198, doi:10.1136/bmj.m1198 (2020).

45. Grasselli, G., Pesenti, A. & Cecconi, M. Critical Care Utilization for the COVID-19 Outbreak in Lombardy, Italy: Early Experience and Forecast During an Emergency Response. JAMA, doi:10.1001/jama.2020.4031 (2020).

46. Fox, S. E. et al. Pulmonary and Cardiac Pathology in Covid-19: The First Autopsy Series from New Orleans. medRxiv, 2020.2004.2006.20050575, doi:10.1101/2020.04.06.20050575 (2020).

47. Chen, T. et al. Clinical characteristics of 113 deceased patients with coronavirus disease 2019: retrospective study. BMJ 368, m1091, doi:10.1136/bmj.m 1091 (2020).

48. Bhatraju, P. K. et al. Covid-19 in Critically Ill Patients in the Seattle Region – Case Series. New England Journal of Medicine, doi: 10.1056/NEJMoa2004500 (2020).

49. Liu, Y. et al. Viral dynamics in mild and severe cases of COVID-19. The Lancet. Infectious diseases, doi:10.1016/S1473-3099(20)30232-2 (2020).

50. Zhou, S., Zhu, T., Wang, Y. & Xia, L. Imaging features and evolution on CT in 100 COVID-19 pneumonia patients in Wuhan, China. Eur Radiol, doi:10.1007/s00330-020-06879-6 (2020).

51. Stower, H. Virological assessment of SARS-CoV-2. Nature Medicine 26, 465-465, doi:10.1038/s41591-020-0848-x (2020).

52. Magro, C. et al. Complement associated microvascular injury and thrombosis in the pathogenesis of severe COVID-19 infection: A report of five cases. Translational Research, (Apr. 15, 2020).

53. Gao, T. et al. Highly pathogenic coronavirus N protein aggravates lung injury by MASP-2-mediated complement over-activation. medRxiv, 2020.2003.2029.20041962, doi: 10.1101/2020.03.29.20041962 (2020).

54. Risitano, A. M. et al. Complement as a target in COVID-19? Nature reviews. Immunology, doi: 10.1038/s41577-020-0320-7 (2020).

55. Diurno, F. et al. Eculizumab treatment in patients with COVID-19: preliminary results from real life ASL Napoli 2 Nord experience. Eur Rev Med Pharmacol Sci 24, 4040-4047, doi:10.26355/eurrev-202004-20875 (2020).

56. Qin, X. et al. Glycation inactivation of the complement regulatory protein CD59: a possible role in the pathogenesis of the vascular complications of human diabetes. Diabetes 53, 2653-2661, doi:10.2337/diabetes.53.10.2653 (2004).

57. Ghosh, P. et al. Glycation of the complement regulatory protein CD59 is a novel biomarker for glucose handling in humans. J Clin Endocrinol Metab 99, E999-E1006, doi:10.1210/jc.2013-4232 (2014).

58. Davies, C. S., Harris, C. L. & Morgan, B. P. Glycation of CD59 impairs complement regulation on erythrocytes from diabetic subjects. Immunology 114, 280-286, doi:10.1111/j.1365-2567.2004.02086.x (2005).

59. Cheng, Y. & Gao, M. The effect of glycation of CD59 on complement-mediated cytolysis. Cell Mol Immunol 2, 313-317 (2005).

60. Hertle, E. et al. Distinct Longitudinal Associations of MBL, MASP-1, MASP-2, MASP-3, and MAp44 With Endothelial Dysfunction and Intima-Media Thickness: The Cohort on Diabetes and Atherosclerosis Maastricht (CODAM) Study. Arterioscler Thromb Vasc Biol 36, 1278-1285, doi:10.1161/ATVBAHA.115.306552 (2016).

61. Nelson, R. A., Jr. The immune-adherence phenomenon; an immunologically specific reaction between microorganisms and erythrocytes leading to enhanced phagocytosis. Science 118, 733-737, doi:10.1126/science.118.3077.733 (1953).

62. Sun, S. et al. Inhibition of complement activation alleviates acute lung injury induced by highly pathogenic avian influenza H5N1 virus infection. Am J Respir Cell Mol Biol 49, 221-230, doi:10.1165/rcmb.2012-04280C (2013).

63. Sun, S. et al. Treatment with anti-C5a antibody improves the outcome of H7N9 virus infection in African green monkeys. Clin Infect Dis 60, 586-595, doi:10.1093/cid/ciu887 (2015).

64. Garcia, C. C. et al. Complement C5 activation during influenza A infection in mice contributes to neutrophil recruitment and lung injury. PLoS One 8, e64443, doi:10.1371/journal.pone.0064443 (2013).

65. Gavriilaki, E. & Brodsky, R. A. Severe COVID-19 infection and thrombotic microangiopathy: success doesn’t come easily. Br J Haematol, doi:10.1111/bjh.16783 (2020).

66. Gralinski, L. E. et al. Complement Activation Contributes to Severe Acute Respiratory Syndrome Coronavirus Pathogenesis. mBio 9, doi:10.1128/mBio.01753-18 (2018).

67. Litvinov, R. I. & Weisel, J. W. Role of red blood cells in haemostasis and thrombosis. ISBTscience series 12, 176-183, doi:10.1111/voxs.12331 (2017).

All publications cited in this specification are incorporated herein by reference in their entireties. U.S. Provisional Pat. Application No. 63/022,181, filed May 8, 2020, is incorporated herein by reference in its entirety. Similarly, the SEQ ID NOs which are referenced herein and which appear in the Sequence Listing are incorporated by reference. While the invention has been described with reference to particular embodiments, it will be appreciated that modifications can be made without departing from the spirit of the invention. Such modifications are intended to fall within the scope of the appended claims.

Sequence Listing Free Text

| | | |
|---|---|---|
| SEQ ID NO | <213> | <223> |
| 5-6 | artificial | universal 16S primer |
| 13-46 | artificial | primer |
| 48-49 | artificial | primer |

1. A method of diagnosing a pathogenic infection in a subject, the method comprising
contacting a red blood cell-containing sample from the subject with a reagent capable of detecting a pathogen-associated molecule in the sample; and
diagnosing the subject with a pathogenic infection when the pathogen-associated molecule is detected in the sample, wherein the sample is 10_{µ}L or less.

2. (canceled)

3. The method according to claim 1, wherein the pathogen is a bacteria, virus, mycobacterium, parasite, or plasmodium.

4. The method according to claim 1, wherein the reagent comprises multiple reagents, each capable of detecting a different specific pathogen-associated molecule.

5. The method according to claim 1, wherein the reagent comprises primers capable of binding pathogenic DNA or RNA.

6. The method according to claim 1, wherein the specific pathogen found in the sample is identified.

7. The method according to claim 1, wherein the sample contains no culturable pathogens.

8. The method according to claim 1, wherein the method further comprises treating the subject for the pathogenic infection, when diagnosed with the same.

9. (canceled)

10. The method according to claim 1, further comprising filtering the blood sample to remove non-RBC blood cells.

11-12. (canceled)

13. The method according to claim 1, wherein the sample contains at least 1 million RBC.

14-15. (canceled)

16. The method according to claim 1, wherein the subject is suspected of having sepsis or pneumonia.

17-20. (canceled)

21. The method according to claim 1, wherein the reagent is capable of detecting 16S ribosomal DNA.

22. The method according to claim 21, wherein the reagent is capable of detecting the V4 region of 16S.

23. The method according to claim 1, wherein the reagent comprises primers capable of binding the bacterial DNA.

24-25. (canceled)

26. The method according to claim 1, wherein the pathogenic infection is a viral infection, and wherein the reagent is capable of detecting viral DNA or RNA in the sample; and the subject is diagnosed with a viral infection when viral DNA or RNA is detected in the sample.

27-33. (canceled)

34. The method according to claim 1, wherein the pathogenic infection is a parasitic infection, and wherein the reagent is capable of detecting parasite DNA in the sample; and the subject is diagnosed with a parasitic infection when parasite DNA is detected in the sample.

35. The method according to claim 34, wherein the subject is culture-negative for parasitic infection.

36. The method according to claim 34, further comprising treating the subject for the parasitic infection when diagnosed with the same.

37. (canceled)

38. The method according to claim 34, wherein the reagent comprises primers capable of binding the parasite DNA.

39-40. (canceled)

41. The method according to claim 1, wherein the pathogenic infection is a fungal infection, and wherein the reagent is capable of detecting fungal DNA in the sample; and the subject is diagnosed with a fungal infection when fungal DNA is detected in the sample.

42-46. (canceled)

47. A method of detecting complement activation in a subject suspected of having a pathogenic infection, comprising contacting a RBC containing sample with a reagent capable of identifying a complement protein, or fragment thereof, and diagnosing the subject with compliment activation when a compliment protein or fragment thereof is detected on the RBCs, and treating the subject for compliment activation when the subject is diagnosed with the same.

48-52. (canceled)


SEQUENCE LISTING (49 sequences)

SEQ ID NO: 1 - DNA, 27 bp, Plasmodium falciparum
agcggttaacctttctttttccttacg

SEQ ID NO: 2 - DNA, 29 bp, Plasmodium falciparum
agtgcatcatgtatgacagcatgtttaca

SEQ ID NO: 3 - DNA, 21 bp, Homo sapiens
tgatctgctgcagtgctctga

SEQ ID NO: 4 - DNA, 19 bp, Homo sapiens
tcaggccacctacggtgaa

SEQ ID NO: 5 - DNA, 20 bp, Artificial Sequence
  universal 16s primer
agagtttgatcctggctcag

SEQ ID NO: 6 - DNA, 15 bp, Artificial Sequence
  universal 16s primer
ctgctgcctyccgta

SEQ ID NO: 7 - DNA, 20 bp, Mus musculus
gccccagatatagcattccc

SEQ ID NO: 8 - DNA, 20 bp, Mus musculus
gttcatcctgttcctgctcc

SEQ ID NO: 9 - DNA, 24 bp, Homo sapiens
tcgtcgttttgtcgttttgtcgtt

SEQ ID NO: 10 - DNA, 20 bp, Mus musculus
tccatgacgttcctgacgtt

SEQ ID NO: 11 - DNA, 17 bp, Plasmodium falciparum
tcgtcgtcgtcgtcgtc

SEQ ID NO: 12 - DNA, 21 bp, Homo sapiens
ggggagcagctgctggggggg

SEQ ID NO: 13 - DNA, 19 bp, Artificial Sequence
  Primer
gtgccagcmgccgcggtaa

SEQ ID NO: 14 - DNA, 19 bp, Artificial Sequence
  Primer
gtgccagcmgccgcggtaa

SEQ ID NO: 15 - DNA, 20 bp, Artificial Sequence
  Primer
agagtttgatcmtggctcag

SEQ ID NO: 16 - DNA, 19 bp, Artificial Sequence
  Primer
ggttaccttgttacgactt

SEQ ID NO: 17 - DNA, 21 bp, Artificial Sequence
  Primer
gagagtttgatcctggctcag

SEQ ID NO: 18 - DNA, 20 bp, Artificial Sequence
  Primer
ctacggctaccttgttacga

SEQ ID NO: 19 - DNA, 17 bp, Artificial Sequence
  Primer
attaccgcggctgctgg

SEQ ID NO: 20 - DNA, 18 bp, Artificial Sequence
  Primer
  misc_feature (10)..(10): n is a, c, g, or t
cctacggggnggcwgcag

SEQ ID NO: 21 - DNA, 21 bp, Artificial Sequence
  Primer
gactachvgggtatctaatcc

SEQ ID NO: 22 - DNA, 19 bp, Artificial Sequence
  Primer
gtgccagcmgccgcggtaa

SEQ ID NO: 23 - DNA, 19 bp, Artificial Sequence
  Primer
gtgycagcmgccgcggtaa

SEQ ID NO: 24 - DNA, 20 bp, Artificial Sequence
  Primer
ggactachvgggtwtctaat

SEQ ID NO: 25 - DNA, 19 bp, Artificial Sequence
  Primer
  misc_feature (8)..(8): n is a, c, g, or t
ggactacnvgggtwtctaa

SEQ ID NO: 26 - DNA, 19 bp, Artificial Sequence
  Primer
gtgccagcmgccgcggtaa

SEQ ID NO: 27 - DNA, 19 bp, Artificial Sequence
  Primer
gtgycagcmgccgcggtaa

SEQ ID NO: 28 - DNA, 20 bp, Artificial Sequence
  Primer
ggactachvgggtwtctaat

SEQ ID NO: 29 - DNA, 20 bp, Artificial Sequence
  Primer
tcggmtcgtaaaactctgtt

SEQ ID NO: 30 - DNA, 19 bp, Artificial Sequence
  Primer
ctgctggcacgaagttagc

SEQ ID NO: 31 - DNA, 28 bp, Artificial Sequence
  Primer
aagaacatatgtgtaagtaactgtgcac

SEQ ID NO: 32 - DNA, 18 bp, Artificial Sequence
  Primer
gccttcgggttgtaaagy

SEQ ID NO: 33 - DNA, 19 bp, Artificial Sequence
  Primer
ctgctggcacgaagttagc

SEQ ID NO: 34 - DNA, 25 bp, Artificial Sequence
  Primer
ttaataacctcgattgacgttaccc

SEQ ID NO: 35 - DNA, 22 bp, Artificial Sequence
  Primer
tacctacccttgacatacagtg

SEQ ID NO: 36 - DNA, 18 bp, Artificial Sequence
  Primer
cttcctccggtttgtcac

SEQ ID NO: 37 - DNA, 19 bp, Artificial Sequence
  Primer
gagtccccaccatcacatg

SEQ ID NO: 38 - DNA, 20 bp, Artificial Sequence
  Primer
ctggaagcaggatggctatt

SEQ ID NO: 39 - DNA, 22 bp, Artificial Sequence
  Primer
cagtagcgggaagagaatgtag

SEQ ID NO: 40 - DNA, 24 bp, Artificial Sequence
  Primer
aactgctcttccaccgacaacgac

SEQ ID NO: 41 - DNA, 26 bp, Artificial Sequence
  Primer
gctgttttagcagatagtgagatcga

SEQ ID NO: 42 - DNA, 18 bp, Artificial Sequence
  Primer
tcccagtcggtgctgtca

SEQ ID NO: 43 - DNA, 20 bp, Artificial Sequence
  Primer
aatgttacgcaactgacgag

SEQ ID NO: 44 - DNA, 19 bp, Artificial Sequence
  Primer
agagttgatcctggctcag

SEQ ID NO: 45 - DNA, 15 bp, Artificial Sequence
  Primer
ctgctgcctyccgta

SEQ ID NO: 46 - DNA, 17 bp, Artificial Sequence
  Primer
taacacatgcaagtcga

SEQ ID NO: 47 - RNA, 20 nt, Human immunodeficiency virus
gcccgucuguugugugacuc

SEQ ID NO: 48 - DNA, 20 bp, Artificial Sequence
  Primer
gtgaaattgttgaaagggaa

SEQ ID NO: 49 - DNA, 18 bp, Artificial Sequence
  Primer
gactccttggtccgtgtt